(19) United States
(12) Patent Application Publication
(10) Pub. No.: US 2004/0106198 A1
(43) Pub. Date: Jun. 3, 2004

(54) Inhibition of peptide cleavage in plants

(75) Inventors: Kathleen M. Hanley, Vacaville, CA (US); Fakhrieh S. Vojdani, Davis, CA (US); Long V. Nguyen, Vacaville, CA (US); Wayne P. Fitzmaurice, Vacaville, CA (US)

Correspondence Address: LARGE SCALE BIOLOGY CORPORATION, 3333 VACA VALLEY PARKWAY, SUITE 1000, VACAVILLE, CA, 95688, US

(73) Assignee: LARGE SCALE BIOLOGY CORPORATION, Vacaville, CA

(21) Appl. No.: 10/620,669

(22) Filed: Jul. 16, 2003

Related U.S. Application Data
(60) Provisional application No. 60/396,396, filed on Jul. 16, 2002.

(51) Int. Cl. C12N 5/04

(52) U.S. Cl. 435/419

(57) ABSTRACT

The invention provides a system for expressing a foreign peptide in a plant cell, wherein the foreign protein is sensitive to a protease activity in the plant cell, by introducing into a plant cell a polynucleotide, which encodes the foreign protein and an another polynucleotide, which encodes a genetic element capable of reducing the protease activity in the plant cell. The invention also provides for plant cells, which incorporate this system, and for methods of reducing the proteolysis of the foreign protein expressed in a plant cell by using this system.

### CROSS REFERENCE TO RELATED APPLICATIONS

This application claims benefit of the filing date of U.S. Provisional Application No. 60/396,396 filed Jul. 16, 2002 and is incorporated herein by reference.

### FIELD OF THE INVENTION

This present invention is related to the field of plant molecular biology and expression of a protein in a plant host. In particular, this invention relates to the inhibition of peptide cleavage of the protein by a protease native to the plant host. The inhibition is effected by the use of a recombinant nucleic acid, which is inserted into a heterologous virus or by plant transformation to inhibit the protease activity in the plant host.

### BACKGROUND OF THE INVENTION

Proteolytic enzymes or proteases are enzymes that function by catalyzing the cleavage of peptide bonds in proteins. Proteases are ubiquitous in nature and are involved with both random and site-specific cleavage of peptide bonds. Table 1 lists the major families of proteolytic enzymes and their corresponding active site residues (Neurath, 1984). Proteolytic function is determined in part by the structural arrangement of these amino acid residues.

TABLE 1
Families of proteolytic enzymes

| Family¹ | Representative protease(s) | Active site residues² |
|---|---|---|
| A. Serine Protease I | Chymotrypsin (EC 3.4.21.1) | Asp¹⁰², Ser¹⁹⁵, |
| | Trypsin (EC 3.4.21.1) | His⁵⁷ |
| | Elastase (EC 3.4.21.11) | |
| | Pancreatic Kallikrein | |
| | (EC 3.4.21.8) | |
| B. Serine Protease II | Subtilisin (EG 3.4.21.14) | Asp³², Ser²²¹, His⁶⁴ |
| C. Cysteine | Papain (EC 3.4.22.2) | Cys²⁵, His¹⁵⁹, |
| proteases | Actinidin | Asp¹⁵⁸ |
| | Rat liver cathepsins B & H | |
| D. Aspartic Protease | Penicillopensin (EC 3.4.23.6) | Asp³³, Asp²¹³ |
| | Rhizopus Chineses, acid | |
| | protease | |
| | Endothia Parasitica, acid | |
| | proteases | |
| | Rennin (EC 3.4.99.19) | |
| | Pepsin (EC 3.4.23.1) | |
| | Chymosin (EC 3.4.23.4) | |
| E. Metallo-Protease | Bovine carboxypeptidase A | Zn, Glu²⁷⁰, Try²⁴⁸ |
| I | (EC 3.4.17.1) | |
| F. Metallo-Protease | Thermolysin (EC 3.4.24.4) | Zn, Glu¹⁴³, His²³¹ |
| II | | |

¹This table includes only enzymes of known amino acid sequence and three dimensional structure, except for rat liver cathepsin B and H, for which the three dimensional structure has been surmised by analogy to papain.
²The residue number corresponds to the amino acid sequence of the enzymes listed in bold in column 2.

Chymotrypsin- and subtilisin-like serine proteases are the largest groups of the serine protease family. The arrangement of the amino acid residues aspartate, histidine and serine in the catalytic triad is highly conserved in both, with differences occurring primarily in the protein scaffolding (Siezen and Leunissen, 1997). Proteases, in general, exist as pre-proteins that are functionally inactive until cleavage of the targeting peptide (Neurath, 1984). Subtilisin-like serine proteases exist as pre-proenzymes (Gensberg, Jan, and Matthews, 1998; Neurath, 1984; Siezen and Leunissen, 1997). The pre-peptide (or signal peptide) acts as a targeting sequence to direct the proenzyme outside the cell via the secretory pathway and cleavage of the pro-peptide results in the active protease. The prodomain of subtilisin-like proteases has been implicated in serving specific regulatory functions associated with the protease. These include, chaparonin-like folding properties (Creemers, Jackson, and Hutton, 1998; Gensberg, Jan, and Matthews, 1998; Yamagata et al., 1994), correct temporal and spatial activation (Creemers, Jackson, and Hutton, 1998; Tomero, Conejero, and Vera, 1996; Yamagata et al., 1994), and intracellular transport, stability and sorting (Creemers, Jackson, and Hutton, 1998; Gensberg, Jan, and Matthews, 1998).

Mammalian subtilisin-like serine proteases are known to be involved in the processing of numerous biologically important prohormones and proproteins (Barr, 1991; Creemers, Jackson, and Hutton, 1998; Gensberg, Jan, and Matthews, 1998; Steiner et al., 1992). These proteases act within the secretory pathway to cleave specific basic amino acid residues thus generating the active molecule (Gensberg, Jan, and Matthews, 1998). The most common processing sites found in mammalian subtilisin-like proteases are pairs of basic amino acid residues Lys:Arg and Arg:Arg, but cleavage motifs including mono-, tri-, tetra- and pentabasic residues have also been characterized (Barr, 1991). Some examples of proteins catalyzed by mammalian subtilisin-like proteases include; nerve growth factor, proinsulin C, insulin proreceptor, proalbumin, and prorenin.

Human growth hormone (hGH), a major protein of the pituitary gland and involved in numerous regulatory functions (Sinha and Jacobsen, 1994), exists as a prohormone and is processed in vivo, giving rise to catalytically active peptide fragments (Creemers, Jackson, and Hutton, 1998; Salem, 1988; Sinha and Jacobsen, 1994). Proteolytic processing can occur in an exposed domain of the large disulfide region of the hGH protein (Gellerfors et al., 1990; Wroblewski, Kaiser, and Becker, 1993). An in vitro study using biosynthetic hGH and thyroid gland extracts rich in a protease that was similar to a chymotrypsin-like serine protease showed metabolic intermediates were formed exclusively by cleavage carboxy to the tyrosine, phenylalanine or leucine amino acid residues (Wroblewski, Kaiser, and Becker, 1993).

Stem cell factor from various species and variants are known to have a variety of desirable biological activities. See Zhang et al, Biology of Reproduction 50:95-102, Davis et al, Cytokine 9(4): 263-275 (1997), WO 96/18726 and WO 97/38101.

Plant subtilisin-like serine protease genes (Ribeiro et al., 1995; Tornero, Conejero, and Vera, 1996; Yamagata et al., 1994), have been isolated and have been subgrouped into the Pyrolysin family of subtilisin-like proteases (Siezen and Leunissen, 1997). As is typical of subtilisin-like proteases (Barr, 1991; Gensberg, Jan, and Matthews, 1998), plant subtilisin-like genes also encode proteins that are synthesized as pre-proenzymes. These proteases have been implicated in many different aspects of plant development. Tissue specific gene expression has been reported in pollen (Taylor et al., 1997), fruit (Rudenskaya et al., 1995) etc. Specific plant proteases and protease inhibitors are induced as part of a cascade of defense-related activities (Tornero, Conejero, and Vera, 1996; Tomero, Conejero, and Vera, 1997). Additionally, studies using reporter gene constructs to the promoters of two pathogen-induced tomato subtilisin-like protease genes revealed induced reporter gene activity following challenge with either Pseudomonas syringae or salicylic acid (Taylor et al., 1997).

Plant subtilisin-like proteases have broad substrate specificity (Siezen and Leunissen, 1997). Unlike mammalian proteases, the plant subtilisin-like proteases studied to date prefer cleaving amino acids with bulky hydrophobic or aromatic side chains (Yamagata et al., 1994). In vitro studies have determined that processing frequently occurs at sites similar to those targeted by chymotrypsin-like serine proteases (Kaneda, Yonezawa, and Uchikoba, 1995; Wroblewski, Kaiser, and Becker, 1993; Yamagata et al., 1994).

Because plant subtilases have been detected in numerous plant tissues (Ribeiro et al., 1995; Rudenskaya et al., 1998; Rudenskaya et al., 1995; Tomero, Conejero, and Vera, 1996; Tomero, Conejero, and Vera, 1997; Yamagata et al., 1994) the recombinant expression of heterologous sequences in plants may be problematic for some proteins. Some plant proteases may have a high affinity for certain heterologous proteins that are being expressed. In particular, proteins such as human growth hormone, that are proteolytically processed in nature by enzymes with substrate specificities similar to those that have been identified in plants, may be susceptible to degradation in planta or in contact with plant extracts.

### SUMMARY OF THE INVENTION

The present invention provides for a plant cell comprising one or more polynucleotides, wherein the one or more polynucleotides encode a protein of interest and one or more genetic element(s) capable of reducing a protease activity in a plant cell, wherein the polynucleotides are capable of expressing the protein of interest in the plant cell, wherein the protease activity is capable of cleaving the protein of interest, wherein the protein of interest is preferably non-native to the plant cell.

The present invention also provides for a plant cell comprising a non-native polynucleotide, wherein the polynucleotide encodes a protein of interest, wherein the polynucleotide is capable of expressing the protein of interest in the plant cell, wherein the polynucleotide comprises a genetic element capable of reducing a protease activity in the plant cell, wherein the protease activity is capable of cleaving the protein of interest, wherein the protein of interest is preferably non-native to the plant cell.

The present invention further provides for a method of reducing the amount of a protein of interest cleaved by a protease activity in a plant cell, comprising the steps of:

(a) introducing a polynucleotide into a plant cell, wherein the first polynucleotide comprises a genetic element capable of reducing a protease activity in the plant cell; and

(b) expressing a protein of interest in the plant cell, wherein the protein of interest is heterologous to the plant cell, wherein the protein of interest is capable of expression in the plant cell, whereby the amount of protein of interest cleaved by the protease activity in the plant cell is reduced compared to the amount of protein of interest cleaved by the protease activity in another plant cell in which the first polynucleotide is not introduced.

The present invention also provides for a protein of interest, or one or more fragments thereof, produced using the subject plant or plant cell and/or subject method. The present invention further provides for a polynucleotide comprising the genetic element capable of reducing a protease activity in a plant or a plant cell. The present invention further provides for a polynucleotide comprising the (1) coding sequence of a Nicotianalisin protein, or one or more fragments thereof, or (2) a sequence with substantial similarity to one or more conserved region of the Nicotianalisin protein, which is capable of specifically hybridizing to a second polynucleotide encoding a related protease protein for the purpose of identifying the second polynucleotide from a mixture of known or unknown polynucleotides.

Novel plant protease Nicotianalisin and similar protease genes may be cloned per se and then used to produce the active enzyme as a product per se.

### BRIEF DESCRIPTION OF THE FIGURES

FIG. 1 depicts the in vitro stability of expressed recombinant human growth hormone protein (hereafter “hGH”) in the interstitial fluid (hereafter “IF”) following the addition of protease inhibitors. Recombinant hGH protein was expressed in N. benthamiana using a tobacco mosaic virus (hereafter “TMV”) vector expression system. IF was prepared from plant leaves at 9 days post-inoculation and incubated for 24 hours at 24° C. followed by 120 hours at 5° C. with or without the addition of protease inhibitors. Fifteen microliters of the IF extract was separated by SDS-PAGE and the gel prepared for Western analysis. The levels of hGH protein in the IF of the time zero control (lane a), in absence of inhibitor (lane b), and in the presence of protease inhibitors chymostatin (lane c) and PI-I (lane d), were detected using anti-hGH antibody.

FIG. 2 depicts the inhibition of Nicotianalisin by PMSF protease inhibitor using Zymogram gelatin gel analysis. Plant extracts containing Nicotianalisin activity were partially purified by ion-exchange chromatography at pH 5.2, and fractions with peak protease activity were treated with or without 2 mM PMSF for 1 hr at 24° C. Following treatment, aliquots were removed and inhibitory activity assessed on Zymogram gels.

FIG. 3 depicts the SDS-PAGE gel of fractions from N. benthamiana subtilisin-like protease purification. A subtilisin-like protease was purified from virally-infected N. benthamiana leaf IF using column chromatography and 2.5 μg total protein was loaded per lane. Lanes are as follows: (MW) molecular weight markers; (1) plant IF extract; (2) butyl Sepharose; (3) DEAE Sepharose; (4) Sephacryl 100; and, (5) Superose-12. The gel was stained with Coomassie blue.

FIG. 4 depicts the N-terminal sequence comparison of Nicotianalisin and other plant subtilisin-like proteases. The N-terminus of the mature protein of Nicotianalisin purified from N. benthamiana leaves was aligned with eleven other plant subtilisins using DNAMAN Multiple Alignment software (Lynnon BioSoft). A consensus sequence of greater than 75% identity was generated.

FIG. 5 depicts the Nicotianalisin enzyme activity pH optimum. Aliquots of purified Nicotianalisin protease were assayed for proteolytic activity, and the pH optimum was determined. Enzyme assays were conducted at 37° C. in 0.1 M Tris-propane containing 5 mM CaCl₂, pH range from 6 to 10.5. N-Suc-AAPF-pNA SEQ ID NO: (Del Mar et al., 1980; Del Mar et al., 1979) was used as a substrate at 0.3 mM concentration in the reaction mixture. The absorbance of the p-nitroaniline produced was measured at 410 nm. Values are shown as percentages of the maximum activity.

FIG. 6 depicts the proteolytic activity of Nicotianalisin and other plant subtilisin-like proteases (Rudenskaya et al., 1998) on oxidized bovine insulin B chain. An aliquot of the Nicotianalisin protease was mixed with pure insulin B chain protein, incubated for 1 hr at 37° C., and the mass of the cleavage products assayed by MALDI-TOF Mass Spectrometry. The cleavage specificities of Nicotianalisin and other plant subtilisins are indicated.

FIG. 7 depicts the activity of a purified protease, Nicotianalisin, against hGH protein in in vitro assays. An aliquot of Nicotianalisin protease was mixed with purified hGH protein at a 1:50 ratio of protease:substrate, incubated for 10-30 min at 30° C. and peptide fragments analyzed by Coomassie-stained SDS-PAGE. Lanes are as follows (from left to right): (1) molecular weight markers (Invitrogen, Multimark); (2) protease/substrate incubated for 10 min; (3) protease/substrate incubated for 20 min; (4) protease/substrate incubated for 30 min; (5) substrate only incubated for 10 min; (6) substrate only incubated for 20 min; and, (7) substrate only incubated for 30 min.

FIG. 8 depicts the Western blot of hGH cleaved by purified Nicotianalisin protease. An aliquot of Nicotianalisin protease was mixed with purified hGH protein at a 1:50 ratio of protease:substrate and incubated for 10-30 min at 30° C. Lanes are as follows (from left to right): (1) molecular weight markers (Novex Prestained markers); (2) protease/substrate incubated for 10 min; (3) protease/substrate incubated for 20 min; (4) protease/substrate incubated for 30 min; and, (5) substrate only incubated for 30 min, as a control.

FIG. 9 depicts a DNA agarose gel of the products of RT-PCR-amplification of subtilisin-like protease cDNA. Total RNA was isolated from N. benthamiana (Nb) and Arabidopsis thaliana (At) and used as template to RT-PCR amplify a protease cDNA.

FIG. 10 depicts the deduced amino acid sequence alignment of N. benthamiana (Nb) gene fragment contigs, NbP3 and NbP6, SEQ ID NO: 24 and 25, respectively, and the tomato p69A sequence (Tomero, Conejero, and Vera, 1996). The alignment was performed using DNAMAN Multiple Alignment software. Active site residues are in bold and indicated with asterisk. N-linked glycosylation sites are italicized and underlined. The consensus line indicates amino acid residues that are 100% identical in all three sequences.

FIG. 11 depicts the amino acid sequence alignment of Nicotianalisins (SEQ ID NO: 18 to 29, 39 to 40; including both partial and full open reading frames (ORF) based on deduced amino acid sequence of N. benthamiana cDNA clones) and fourteen other subtilisin-like proteases. These proteases are AG12 (Genbank accession #S52769), AIR3 (Genbank accession #AAD12260), ARA12 (Genbank accession #AAC18851), CUSSP (Genbank accession #BAA06905), F22M8.3 (Genbank accession #AAF76468), MDC16.21 (Genbank accession #BAB02339), P69A (Genbank accession #CAA76724), P69B (Genbank accession #CAA76725), P69C (Genbank accession #CAA76726), P69D (Genbank accession #CAA76727), SBT1 (Genbank accession #CAA06999), SBT2 (Genbank accession #CAA07000), SBT3 (Genbank accession #CAA07001), SBT4 (Genbank accession #CAA06998). The alignment was performed using DNAMAN Multiple Alignment software. A consensus sequence was generated from residues identical in greater than 50% of the sequences. The underlined bold residue (at position 125 of the consensus) indicates the putative start of the mature protein. Bold and italicized residues (at positions 161, 237, and 581 of the consensus) indicate the residues involved in the catalytic triad. Sequences of peptides that were isolated and identified from the N. benthamiana IF are shaded on SEQ ID NO: 18 and 19. The alignment was performed using the DNAMAN Multiple Alignment software.

FIG. 12(A) and (B) depict a tobacco mosaic virus (TMV)-based viral vector construct map containing SEQ ID NO: 3 in an antisense (A) or sense (B) orientation. MP—movement protein; CP-coat protein.

FIG. 13 depicts the plant viral vector mediated down regulation of protease activity in inoculated N. benthamiana leaves. The SEQ ID NO: 3 cDNA gene fragment was cloned into a TMV-based plant viral vector in the antisense orientation. The DNA was transcribed, and infectious RNA was used to inoculate N. benthamiana plants. At 10 days post-inoculation the plant IF fraction was harvested and assayed for inhibition of proteolytic activity using substrate-embedded Zymogram gels. Five μl of uninoculated (lane 1), viral vector control-treated (green fluorescent protein (GFP), (lane 2), or viral vector antisense-treated (lane 3) plant IF extracts were separated on a Zymogram gel and analyzed for gel clearing.

FIG. 14 depicts tobacco rattle virus (TRV) RNA2 construct maps containing SEQ ID NO: 3 in the sense or antisense orientation. CP ORF—coat protein; 2b ORF— non-structural protein; PEBV CP SGP—pea early browning virus coat protein subgenomic promoter.

FIG. 15 depicts the accumulation of recombinant hGH protein using a TRV Nicotianalisin silencing vector and a TMV protein expression vector. Two weeks post-sowing N. benthamiana plants were infected with TRV RNA-1 plus RNA-2 containing a 1.2 kb fragment of SEQ ID NO: 3 in the sense orientation. At 9 days post-inoculation, the N. benthamiana plants were infected with a TMV-based expression vector containing the hGH gene. TMV-infected plants were harvested after an additional 10 days. Plant IF extracts were separated by SDS-PAGE and the gel prepared for Western analysis. The immunoblot was probed with polyclonal anti-hGH antibody. 16 μl of IF extract from plants inoculated with TMV-hGH alone (lane 2), TRV silencing construct and TMV-hGH (lane 3), TRV silencing construct alone (lane 4) or buffer alone (lane 5) were loaded per lane and 20 ng of pituitary gland hGH protein (lane 1) were used as a standard.

FIGS. 16A and 16B depict the nucleotide sequence alignment and its phylogenetic tree, respectively, of 15 N. benthamiana subtilisin-like proteases. The bold and italic nucleotides on the consensus sequence indicate the approximate area of the conserved regions A and B. Bold and italic nucleotides on individual SEQ ID indicate the variable region specific to that SEQ ID. Sequences from these regions were chosen as a target for silencing one or more of the Nicotianalisins. FIG. 16B is a phylogenic tree of the sequences. On FIG. 16B, the number next to a line represents the branch length. The alignment was performed using DNAMAN Multiple Alignment software.

FIG. 17 depicts a GENEWARE® plant viral vector containing the replicase, the movement protein and the heterologous gene aprotinin fused to human and porcine Stem Cell Factor containing a His tag. The subsequent cleavage in vivo or in vitro to release the Stem Cell Factor is depicted by way of Kex-2p protease or the like. (Schaller et al, Proc. Nat. Acad. Sci. 91: 11802-11806 (1994))

FIG. 18 depicts a Coomassie blue stained SDS gel of plant extracts proteins where the plants were infected by various GENEWARE® vectors. Each vector contained different foreign genes to be expressed in the plant. Total grind extracts of plants infected with viral vectors expressing hSCF or pSCF with C-terminal HDEL ER-targeting signals are shown. IF extracts from plants infected with viral vectors containing Aprotinin alone, hSCF, pSCF, hSCF with an N-terminal fusion to Aprotinin, and pSCF with an N-terminal fusion to Aprotinin are shown. A control lane containing purified natural aprotinin and a lane of molecular weight standards (unmarked) are included for comparison purposes.

FIG. 19 depicts a Western blot of an SDS-PAGE gel separating various plant extracts proteins where test lanes were from plants infected with a plant viral vector containing and expressing either hSCF or pSCF or derivatives thereof as a heterologous gene. E. coli produced recombinant hSCF, uninfected plants (healthy), a GENEWARE® vector with a gfp gene as the heterologous insert (clone 5) and labeled molecular weight standards are provided as controls for comparison. Antibody against hSCF was used to detect SCF proteins and fragments in the gels.

FIG. 20 depicts a Western blot of various plant extract proteins comparing stem cell factors yield with and without expressing an aprotinin gene. Antibody against human SCF was used to label SCF proteins and their fragments in the gels. The molecular weight of the SCFs and the degradation products are shown.

### DESCRIPTION OF THE SPECIFIC EMBODIMENTS

Definitions and Abbreviations

Virus-based vector or viral vector means an engineered host virus that is capable of expressing a desired protein or trait in a host.

Expression means transcription, translation, protein synthesis, posttranslational modification or any combination of transcription, translation, protein synthesis and posttranslational modification.

Foreign gene means any nucleic acid that is not derived from or extracted from or native to a host into which it is inserted.

Reporter protein means a protein which, when expressed by a viral vector, allows detection of virus-infected cells.

Host means a cell, tissue, organ, or organism capable expressing the ORFs of the subject polynucleotides. This term is intended to include prokaryotic and eukaryotic cells, organs, tissues or organisms, where appropriate. Bacteria, fungi, yeast, animal (cell, tissue, organ, or organism, including human), and plant (cell, tissue, organ, or organism) are examples of a host.

Infection means the ability of a virus to transfer its nucleic acid to a host or introduce a viral nucleic acid into a host, wherein the viral nucleic acid is replicated and viral proteins are synthesized.

ORF or open reading frame means a nucleotide sequence encoding a series of sense codons that lacks a termination codon within it. The ORF may be encoded in any nucleic acid, including DNA or RNA, and the nucleic acid may be any form, including single-stranded or double-stranded. An ORF may encode a peptide that is expressed and may be a gene.

Substantial sequence similarity is present between two nucleic acid or amino acid sequences when the nucleotide or amino acid sequence of a stretch of at least 10 consecutive nucleotides or amino acids of the two are 50% or more identical to each other.

The Invention

The invention provides for a polynucleotide encoding a genetic element capable of directly or indirectly reducing a protease activity in a plant cell.

The invention also provides for a plant cell comprising a first polynucleotide and a second polynucleotide, wherein the first polynucleotide encodes a protein of interest, wherein the first polynucleotide is capable of allowing expression of the protein of interest in the plant cell, wherein the second polynucleotide comprises a genetic element capable of reducing a protease activity in the plant cell, wherein the protease activity is capable of cleaving the protein of interest, wherein the protein of interest is preferably non-native to the plant cell.

The invention also provides for a plant cell comprising a non-native polynucleotide, wherein the polynucleotide encodes a protein of interest, wherein the polynucleotide is capable of allowing expression of the protein of interest in the plant cell, wherein the polynucleotide comprises a genetic element capable of reducing a protease activity in the plant cell, wherein the protease activity is capable of cleaving the protein of interest. The protein of interest is preferably not native to the plant cell.

The genetic element capable of reducing a protease activity in a plant cell or fluids: (1) is or expresses an RNA transcript capable of reducing expression of a protein with the protease activity in the plant cell, (2) induces expression of a protease inhibitor native to the plant cell in the plant cell, (3) is or expresses a protease inhibitor in the plant cell, (4) is or expresses a repressor of the protease gene, (5) is or expresses a nucleic acid or expresses a protein which up or down regulates a different protein which results in the down regulation of the protease gene, (6) induces a site specific mutation in the protease gene, (7) is or induces expression of a triple strand binding nucleic acid which binds at or near the protease gene, 8) is or induces expression of an artificial binder to the protease gene, 9) is or induces expression of a binder for the protein of interest which protects the protein of interest from the action of the protease, 10) is or expresses an agent to reduce mobility of the protease or to retard protease secretion into the IF or 11) induces expression of a protein or nucleic acid which degrades or increases the degradation of the protease. The direct or indirect protease activity inhibitory activity may require additional cellular or fluid components for functioning. Likewise, the protease activity inhibitory activity may act on other cellular or fluid components, which enhance protease activity of the protease enzyme, such as a cofactor.

An RNA transcript capable of reducing expression of a protein may be an antisense transcript, a sense transcript, or a ribozyme. The antisense transcript is antisense to one or more proteases and is able to reduce, silence, or completely shut off expression of the one or more proteases. The sense transcript is sense to one or more proteases and is able to reduce, silence, or completely shut off expression of the one or more proteases. The antisense or sense element comprises a nucleotide sequence that is substantially homologous to the antisense or sense nucleotide sequence of a protease. Preferably, the antisense or sense element comprises the antisense or sense nucleotide sequence of the protease. The ribozyme is able to recognize mRNA encoding the one or more proteases in order to catalyze cleavage of the mRNA, which brings about the reduction, silence, or complete shut off of expression of the one or more proteases. The genetic element can be designed to specifically reduce the protease activity of one or more specific proteases in a plant cell by encoding a polypeptide protease inhibitor. The quantity of protease may also be reduced by a natural or artificial repressor or inducer analog (which does not induce) encoded by the genetic element. The protease gene itself may be mutated or inactivated by a genetic element that elicits a site-specific mutation in or near the protease gene or its regulatory elements. Such site specific mutations may be induced to create a missense codon, a non-sense codon, a frameshift mutation, an insertion in or a deletion of a portion of the protease gene. Such mutagenesis may be formed by the genetic element or its expression product. Reactive chemicals may enhance the mutation of the protease gene.

Antisense or sense RNA may also be polymerized and/or be fused with bulk polynucleotides to adsorb RNA or to bind to DNA where the bulk polynucleotides or polymers of antisense RNA act as insolubilizing, blocking or sequestering agents for binding to the RNA or DNA, thereby inhibiting synthesis of a protease. The RNA may have an additional polynucleotide sequence which binds to natural cellular compounds and structures to further localize it and the corresponding polynucleotides for the protease activity.

The protease can be native or non-native to the plant cell. The protease can be any protein with direct or indirect protease activity. Preferably, the specific proteases belong to one or more class of proteases. More preferably, the specific proteases of each class of protease have nucleic acid and/or amino acid sequence similarity or contain conserved or identical amino acid residues. Preferably, the protease can be any protein with a chymotrypsin-like serine protease or a subtilisin-like serine protease activity. More preferably, the protease can be any protein with a Nicotianalisin protease activity.

Preferably, the protease is a serine protease, or a functional fragment thereof. “Functional fragment” means a peptide comprising the minimum amino acids of the catalytic site of the protease wherein the peptide retains the proteolytic activity of the protease (i.e., all or part of the activity of the wild-type protein). More preferably, the protease is a chymotrypsin-like serine protease or a subtilisin-like serine protease. Even more preferably, the protease is a Nicotianalisin protein.

The protein of interest can be a peptide of virtually any amino acid sequence as long as the protein of interest is capable of being expressed in the host cell. The protein of interest can be a protein sensitive to the protease activity to be reduced. The protein of interest can be a plant or a non-plant protein. Microbial proteins may be proteins of interest, particularly those used for vaccine purposes. The non-plant protein can be an animal protein. The animal protein can be a human protein. Representative examples of such proteins of commercial interest produced in recombinant plants include: human growth hormone, chicken interferon, human single chain antibody, human insulin, human alpha-galactosidase, etc.

The protein of interest may even be a protein beneficial to the plant itself and not particularly for isolation therefrom. Insecticidal proteins such as endotoxins from Bacillus thuringensis or non-BT proteins such as VIP3A, cholesterol oxidases, alpha amylase inhibitors, septic wound response proteins, serine protease inhibitors, trypsin inhibitors, chitinase, Beta-1,3-glucanase, etc, may be produced as pesticides. Note Estruch et al, Nature Biotechnology 15:137-141 (1997) and Ryan, Ann. Rev. Cell. Biol. 3:295-317 (1987). Anti-fungal proteins, e.g. Ye et al, Life Sci. 7;67(7):775-81 (2000), Mitsuhara et al, Mol Plant Microbe Interact. August;13(8):860-8 (2000) may also be likewise used. Protein toxins against mammals and birds may also be used provided that the plant is a non-food crop.

Other desirable traits, such as flower or leaf color, salt tolerance, herbicide resistance, proteins altering secondary metabolite concentration, etc., may also be affected using the techniques of the present invention.

Anti-proteosome function activity is included as a form of inhibiting protease activity. Proteosome destabilization and associated protein degradation are considered a type of protease activity. Inhibitors include organic metabolites such as MG 132 and Lactacystin as well as oligo-leucine based peptides such as Calpin inhibitor III.

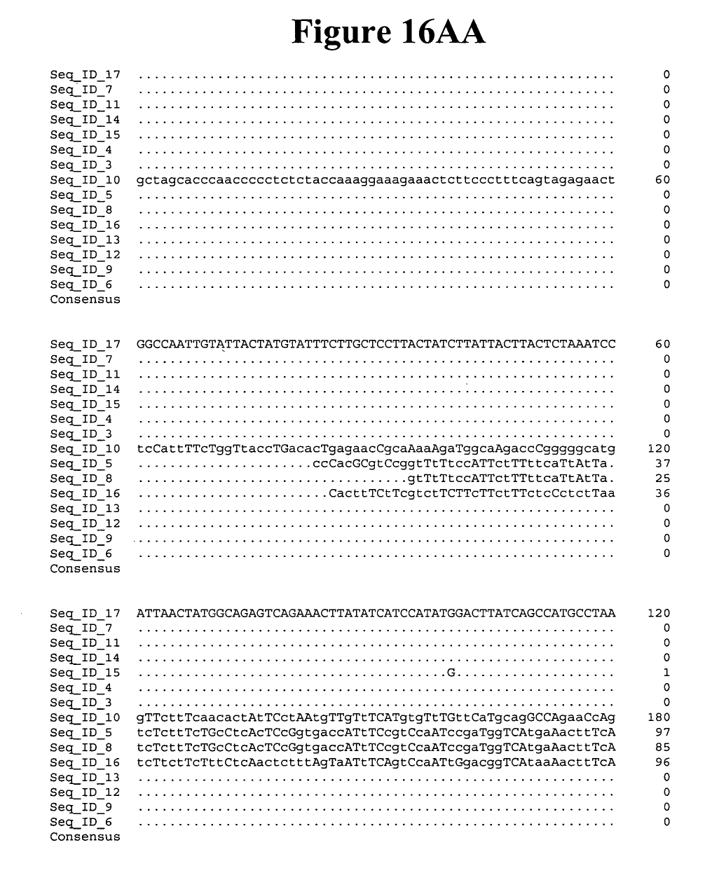

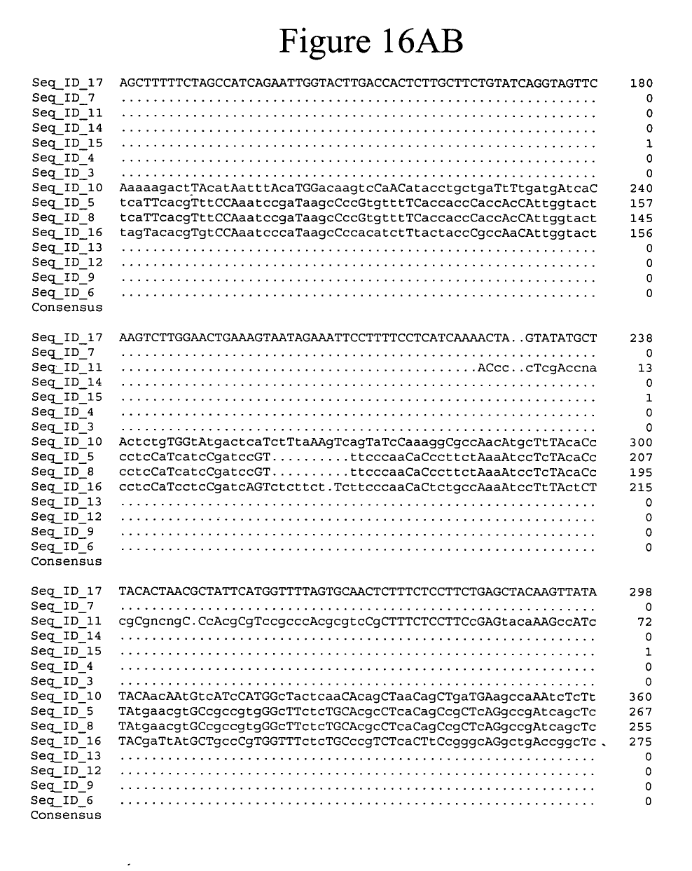

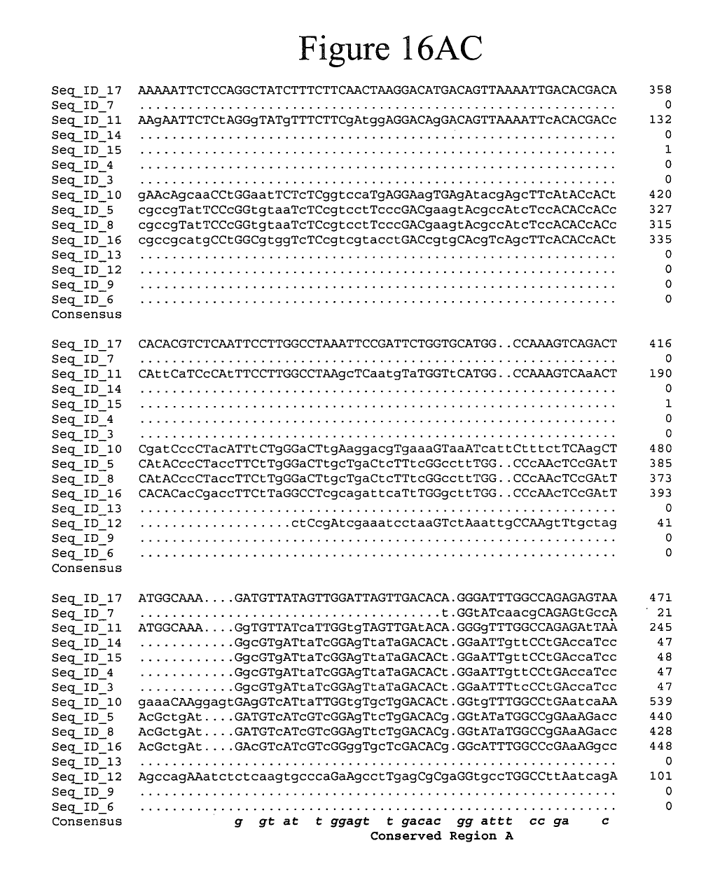

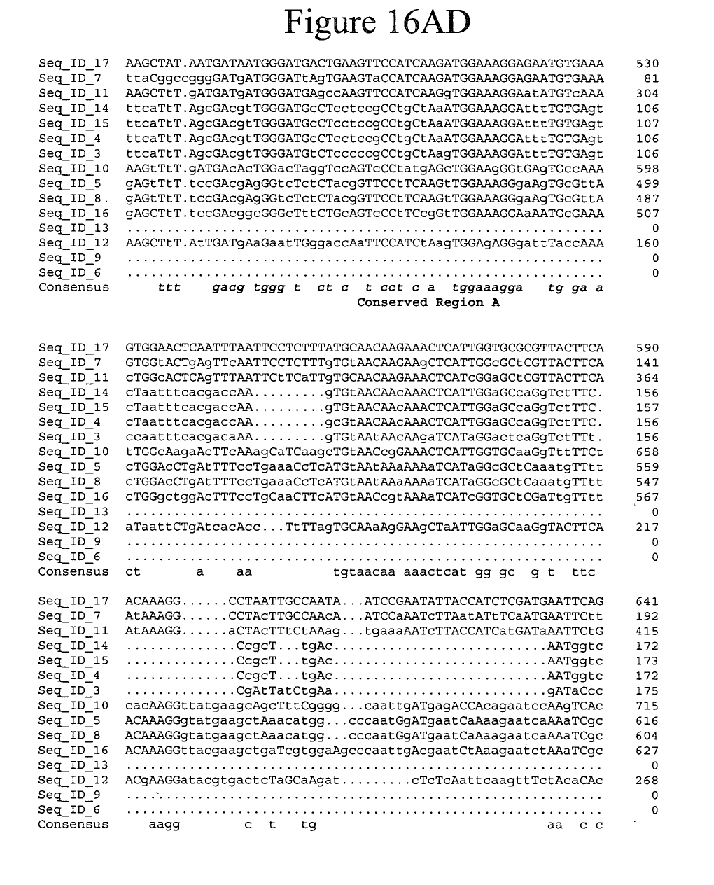

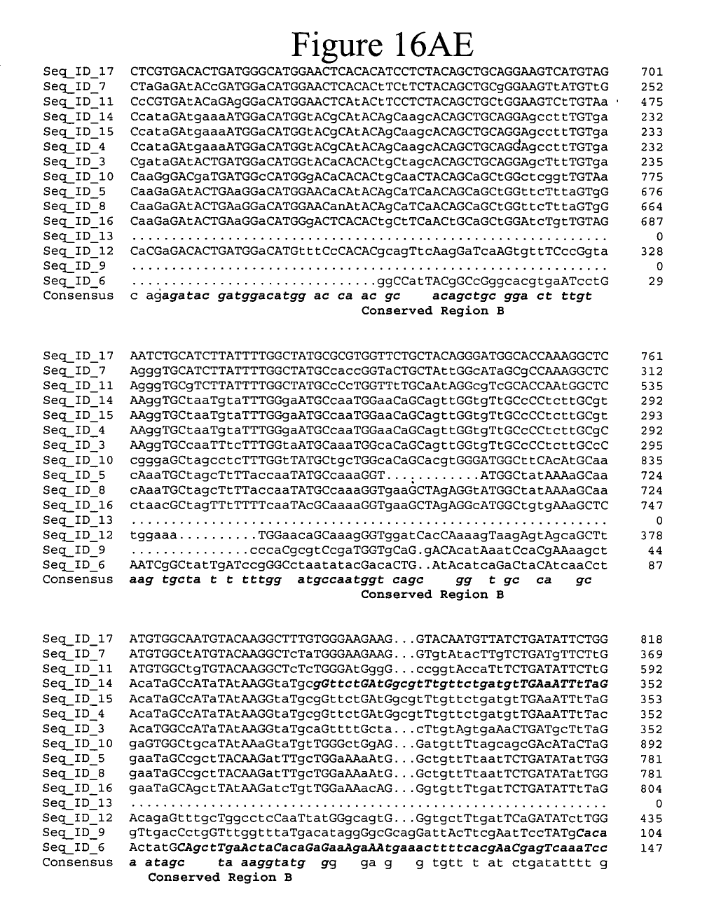

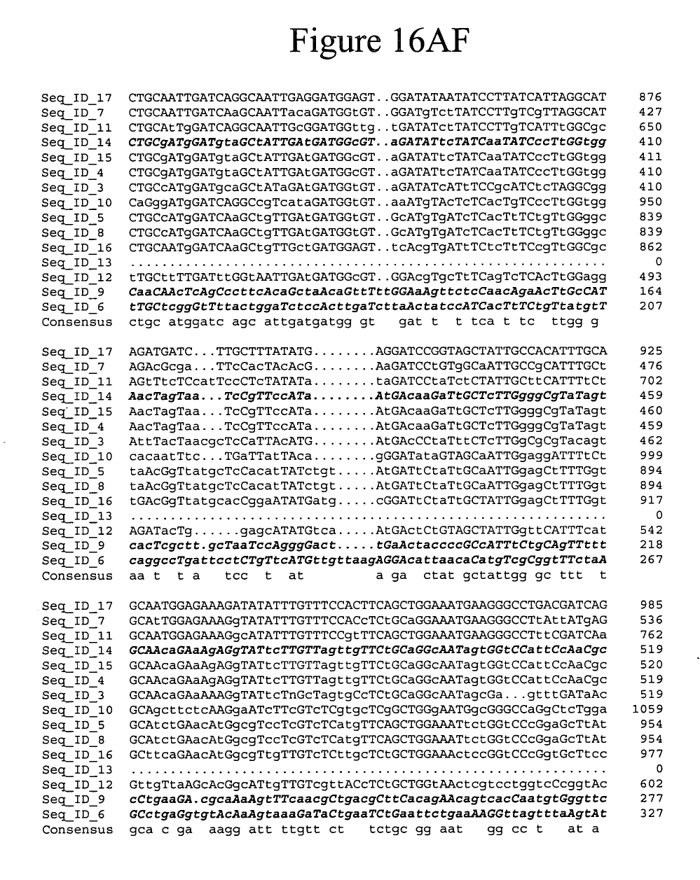

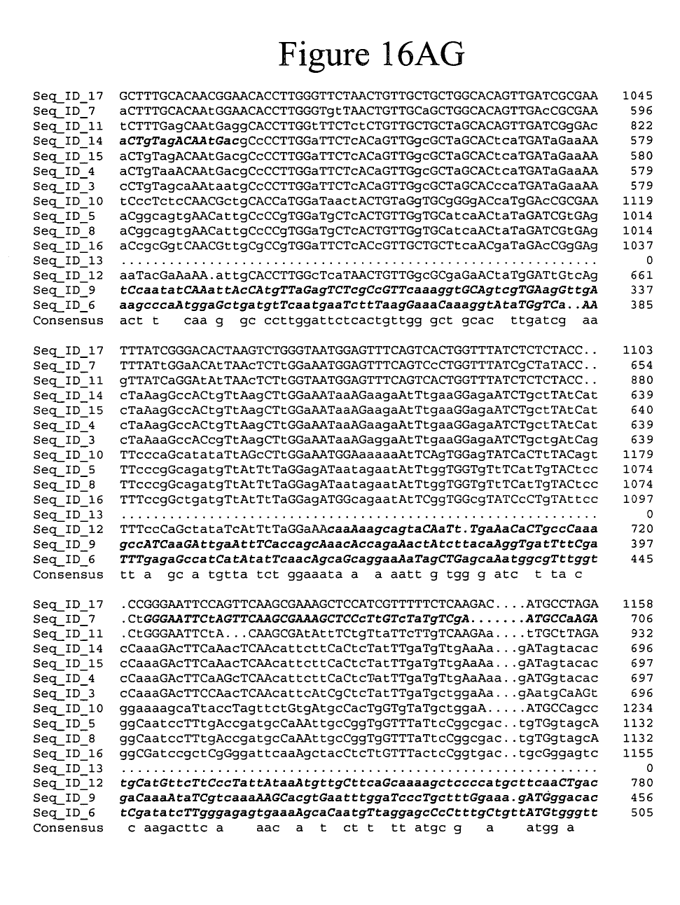

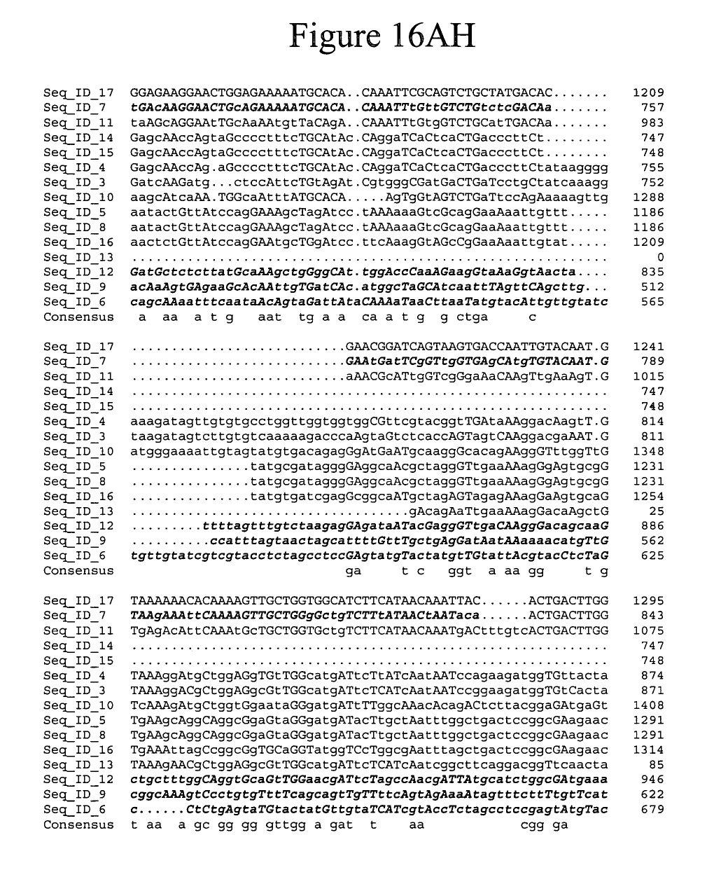

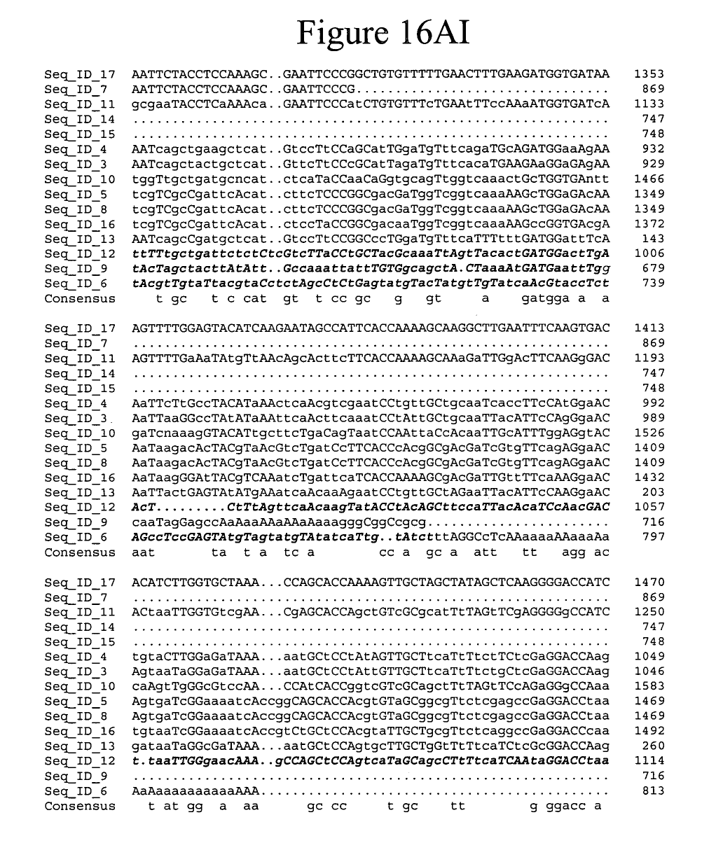

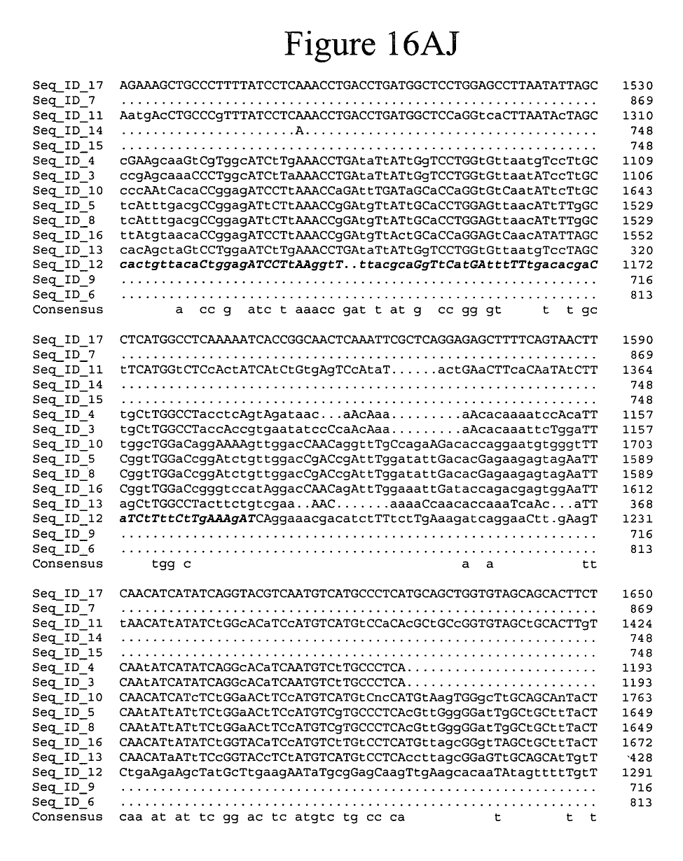

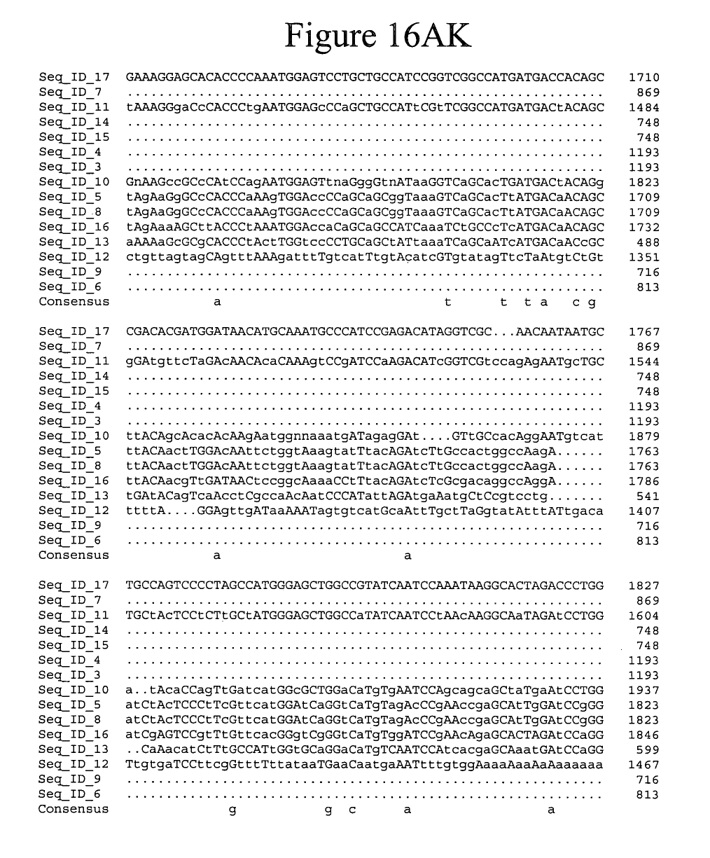

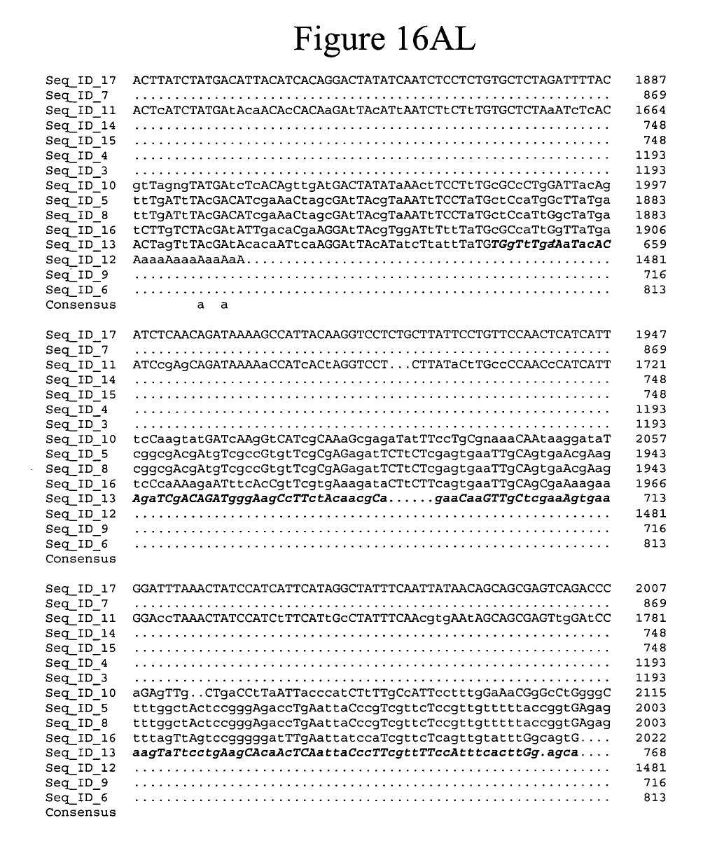

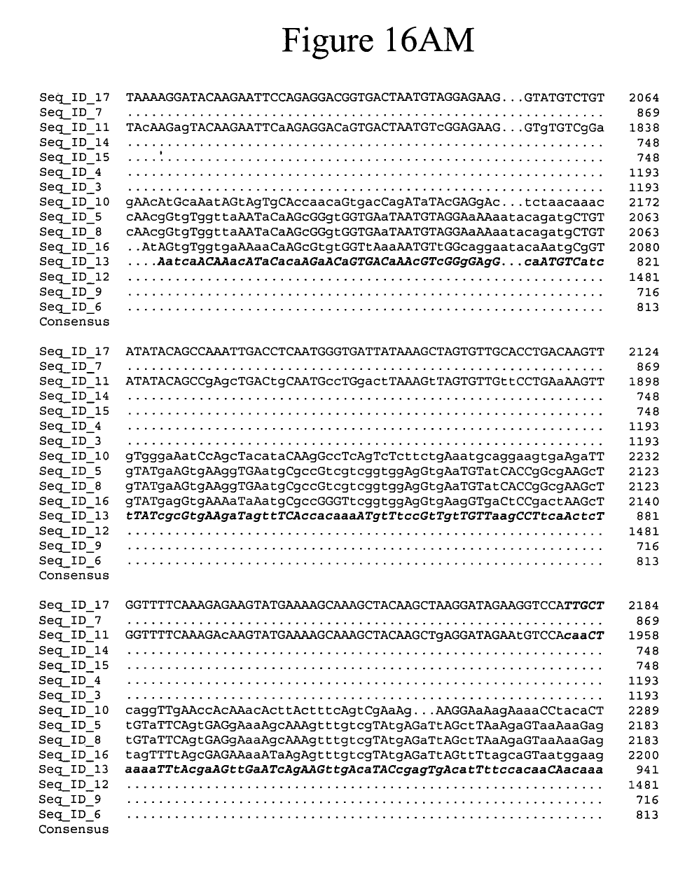

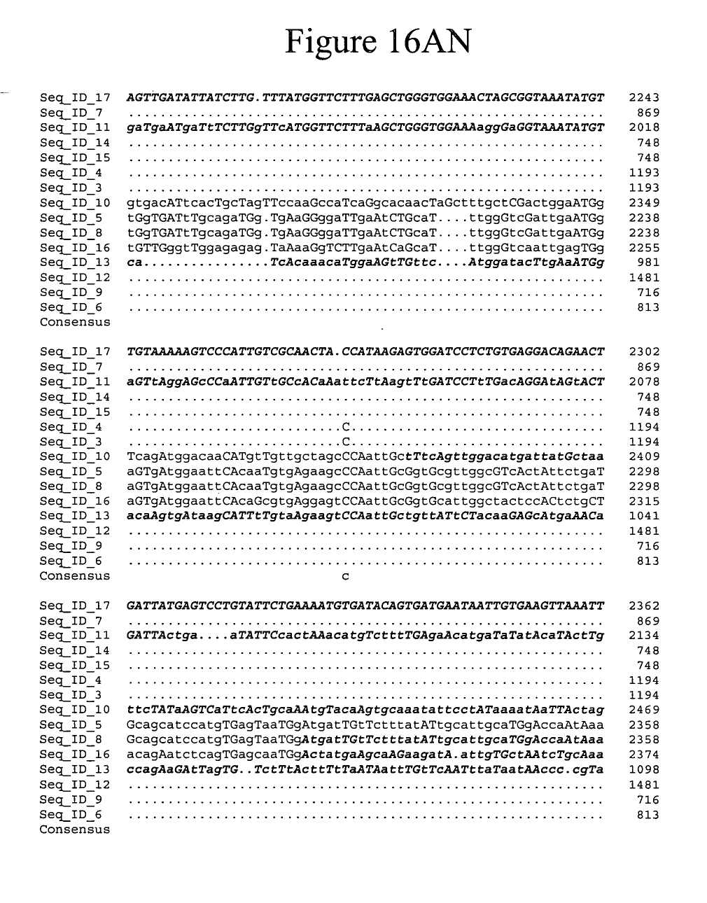

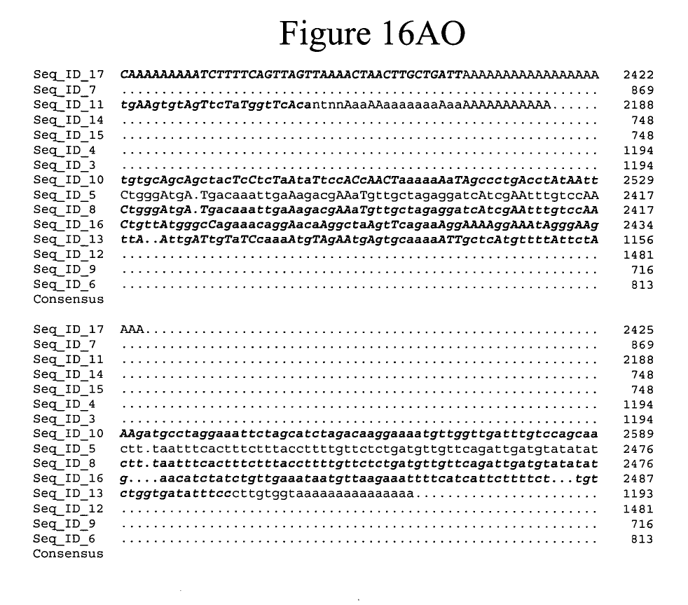

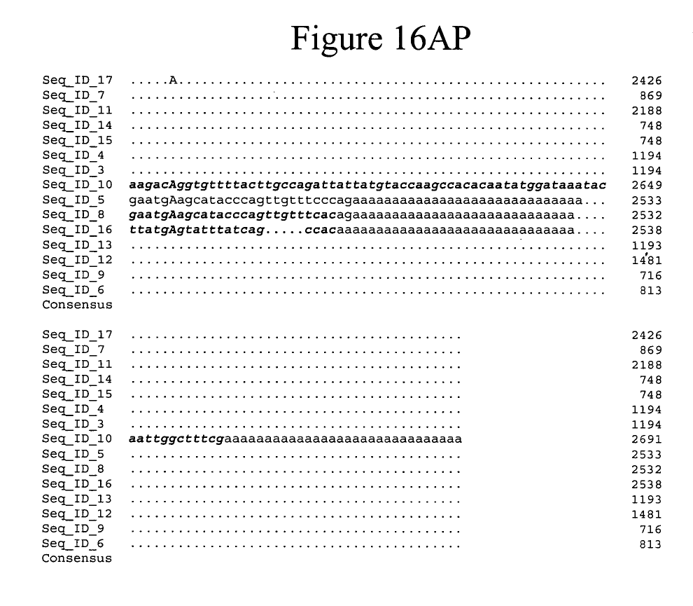

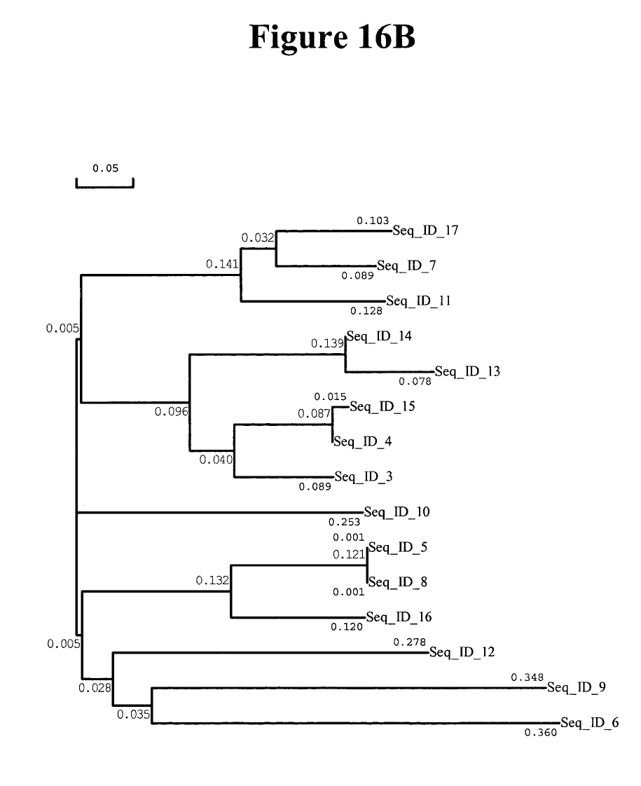

Another aspect of this present invention is a polynucleotide comprising the sequence (or complementary sequence) of a conserved region, or fragment thereof, of the protease mentioned earlier. The sequence (or complementary sequence) of a conserved region is either identical or substantially similar to the conserved regions. FIG. 16 discloses the nucleic acid sequences of such conserved regions. “Substantially similar” means a stretch of nucleotides with sufficient identity so that the sequence is capable of hybridizing to a nucleic acid comprising the conserved sequence. “Substantially similar” can be 50% or more of nucleotide identity. Preferably, it is 70% or more of nucleotide identity. More preferably, it is 80% or more of nucleotide identity. Even more preferably, it is 90% or more of nucleotide identity.

It is recognized that different strains and different species of plants may have slightly different analogous proteases. Furthermore, certain nucleotide changes or even amino acid changes may be employed to alter expression and even to change the protease activity or specificity.

The chemical structures of the inhibitors of protease activity may be diverse. A polypeptide expressed by the genetic element may be such an inhibitor by a number of mechanisms. Alternatively, an RNA expressed by the genetic element may be such an inhibitor by either hybridization, silincing enzymatic functioning or acting as a binding aptamer. A DNA genetic element itself may have the same functions as the RNA to inhibit the protease activity. Any of the polynucleotides mentioned above can be a nucleic acid, a recombinant nucleic acid, a recombinant viral nucleic acid, a genomic nucleic acid component, a subgenomic nucleic acid, a recombinant polynucleotide, or the like. The polynucleotide may be DNA or RNA, either double-stranded (“ds”) or single-stranded (“ss”). ss DNA or ss RNA can be either positive- or plus-sense, or negative- or minus-sense.

The polynucleotide may also comprise synthetic nucleic acid or nucleotides in the stead of DNA or RNA, such as a derivative resistant to degradation in vivo, as discussed below. Within this specification, references to DNA or RNA apply, mutatis mutandis, to other nucleic acids as well, unless clearly forbidden by the context. The bases may be the “normal” bases adenine (A), guanine (G), thymidine (T), cytosine (C) and uracil (U), or abnormal bases such as a synthetic base.

The polynucleotide may be prepared by any desired procedure. The polynucleotide can be synthesized using an automated DNA synthesizer, such as the ABI™ 3900 High-Throughput DNA Synthesizer (Applied Biosystems, Foster City, Calif.).

The polynucleotide may comprise a vector, construct, plasmid, episome, virus, transposon, naked or packaged (e.g. in a liposome) nucleic acid, replicon, or the like. The polynucleotide can be capable of stable replication in one or more of a tissue, a host, a bacterial cell, a prokaryotic cell, an eukaryotic cell, a yeast cell, an animal cell, especially an insect cell, a plant cell, a plant protoplast cell, or the like, for the purpose of amplification. The polynucleotide may be viral and may be recombinant or both. The polynucleotide may comprise a viral or other expression vector or a recombinant expression vector. The polynucleotide can remain extra-chromosomal and need not integrate into any host or organelle chromosome. The polynucleotide can be maintained in the cytoplasm or other compartment of the host and does not need to enter the nucleus of the host and is able to replicate in the cytoplasm or other compartment of the host. The polynucleotide may comprise one or more genomic nucleic acid components, or fragments thereof. The genomic nucleic acid component may comprise a subgenomic nucleic acid or a duplicated subgenomic nucleic acid.

The polynucleotide may or may not be encapsidated by coat protein(s) encoded by the recombinant virus. The polynucleotide may or may not comprise individual features common to certain viruses, such as a cap at the 5′ terminus of the nucleic acid, a specific initial sequence, or a highly conserved 3′ terminus of the nucleic acid. A cap may comprise a 7-methylguanosine cap. A specific initial sequence may comprise an initial sequence of m⁷ GpppGUA. A highly conserved 3′ terminus may comprise a polyadenylate (poly A) sequence that separates the coding region from a 238 nucleotide 3′ terminal tRNA-like structure. The tRNA-like structure may be able to be aminoacylated with tyrosine. The recombinant viral nucleic acid or recombinant virus is used to infect a host. The recombinant nucleic acid is capable of replication in the host, localized or systemic spread in the host, and transcription or expression of the native nucleic acid in the host to express the fusion protein in the host.

The fusion protein product may be cleaved by cellular enzymes to free the desired protein, whether it is a protein of interest of an inhibitor of a protease. Alternatively, the fusion protein may be used by itself as the desired product due to having both activities. For example, a protein of interest may employ a protease inhibitor fused with it as a way for blocking cleavage by the protease by stearic inhibition or by having the inhibitor portion acting on the protease itself. Fusion proteins may also have the added advantage of imparting greater storage stability to a protein of interest.

When the protein of interest is a diagnostic, pharmaceutical, or other directly used protein, a fused protein construct of the protein of interest and another polypeptide may also be used in the same manner. The other polypeptide may be an inhibitor of protease activity or another stabilizer if so desired. Particularly preferred are the use of cleavable linkers, which free the protein of interest before or during use of the protein of interest.

The polynucleotides of the subject invention may be encoded in RNA or DNA or any synthetic nucleic acid, ss or ds, linear or circular, capable of direct or indirect expression into RNA in a eukaryotic host, such as a yeast, such as Sacchromyces cerevisiae, or a prokaryotic host, such as a bacteria, for example Escherichia coli. Depending on the desired host to be used the necessary nucleotide structures necessary for maintenance in the host, such as origin of replication sites, amplifiable selectable markers, etc., and expression in the host, such as promoters, activation sites, etc. need to be present on the RNA or DNA. Such are known to one of ordinary skill in the art (see Old and Primrose, Principles of Gene Manipulation 5th ed., Blackwell Science, Oxford, U.K. (1994) (Old and Primrose, 1994)).

A large number of different vectors and techniques may be used for the present invention. These vectors and techniques are well known per se. The present specification, has focused on viral vectors because of their convenience with plants. However other vectors such as Ti plasmids, transposons, etc. may be used.

Viral vectors into which libraries of genomic or cDNA inserts or sequence variants are inserted may be constructed using a variety of methods known in the art. In preferred embodiments of the instant invention, the viral vectors used to bear such libraries are derived from RNA plant viruses. A variety of plant virus families may be used, such as Bromoviridae, Bunyaviridae, Comoviridae, Geminiviridae, Potyviridae, and Tombusviridae, among others. Within the plant virus families, various genera of viruses may be suitable for the instant invention, such as alfamovirus, ilarvirus, bromovirus, cucumovirus, tospovirus, carlavirus, caulimovirus, closterovirus, comovirus, nepovirus, dianthovirus, furovirus, hordeivirus, luteovirus, necrovirus, potexvirus, potyvirus, rymovirus, bymovirus, oryzavirus, sobemovirus, tobamovirus, tobravirus, carmovirus, tombusvirus, tymovirus, umbravirusa, and among others.

Within the genera of plant viruses, many species are particular preferred. They include alfalfa mosaic virus, tobacco streak virus, brome mosaic virus, broad bean mottle virus, cowpea chlorotic mottle virus, cucumber mosaic virus, tomato spotted wilt virus, carnation latent virus, cauliflower mosaic virus, beet yellows virus, cowpea mosaic virus, tobacco ringspot virus, carnation ringspot virus, soil-borne wheat mosaic virus, tomato golden mosaic virus, cassaya latent virus, barley stripe mosaic virus, barley yellow dwarf virus, tobacco necrosis virus, tobacco etch virus, potato virus X, potato virus Y, rice necrosis virus, ryegrass mosaic virus, barley yellow mosaic virus, rice ragged stunt virus, Southern bean mosaic virus, tobacco mosaic virus, ribgrass mosaic virus, cucumber green mottle mosaic virus watermelon strain, oat mosaic virus, tobacco rattle virus, carnation mottle virus, tomato bushy stunt virus, turnip yellow mosaic virus, carrot mottle virus, among others. In addition, RNA satellite viruses, such as tobacco necrosis satellite may also be employed.

A given plant virus may contain either DNA or RNA, which may be either ss or ds. One example of plant viruses containing ds DNA includes, but not limited to, caulimoviruses such as cauliflower mosaic virus (“CaMV”). Representative plant viruses, which contain ss DNA, are cassaya latent virus, bean golden mosaic virus (“BGMV”), and chloris striate mosaic virus. Rice dwarf virus and wound tumor virus are examples of ds RNA plant viruses. ss RNA plant viruses include tobacco mosaic virus (“TMV”), turnip yellow mosaic virus (“TYMV”), rice necrosis virus (“RNV”), brome mosaic virus (“BMV”), and barley stripe mosaic virus (“BSMV”). The ss RNA viruses can be further divided into plus sense (or positive-stranded), minus sense (or negative-stranded), or ambisense viruses. The genomic RNA of a plus sense RNA virus is messenger sense, which makes the naked RNA infectious. Many plant viruses belong to the family of plus sense RNA viruses. They include, for example, TMV, BMV, BSMV, and others. RNA plant viruses typically encode several common proteins, such as replicase/polymerase proteins essential for viral replication and mRNA synthesis, coat proteins providing protective shells for the extracellular passage, and other proteins required for the cell-to-cell movement, systemic infection and self-assembly of viruses. For general information concerning plant viruses, see Hull, R., Matthews' Plant Virology, 4^{th} Ed., Academic Press, San Diego (2002)(Hull, 2002). The viral genome of the virus can be monopartite (such as tobamovirus), or multipartite, including but not limited to bipartite (such as tobravirus) or tripartite (such as hordeivirus).

Selected groups of suitable plant viruses are characterized below. However, the invention should not be construed as limited to using these particular viruses, but rather the method of the present invention is contemplated to include all plant viruses at a minimum.

### Tobamovirus Group

TMV is a member of the tobamoviruses. The TMV virion is a tubular filament, and comprises coat protein sub-units arranged in a single right-handed helix with the ss RNA intercalated between the turns of the helix. TMV infects tobacco as well as other plants. TMV is transmitted mechanically and may remain infective for a year or more in soil or dried leaf tissue. The TMV virions may be inactivated by subjection to an environment with a pH of less than 3 or greater than 8, or by formaldehyde or iodine. Preparations of TMV may be obtained from plant tissues by (NH₄)₂SO₄ precipitation, followed by differential centrifugation.

TMV is a positive-stranded ssRNA virus whose genome is 6395 nucleotides long and is capped at the 5′-end but not polyadenylated. The genomic RNA contains a short 5′ NTR followed by an ORF of 4848 nucleotides, which includes an amber stop codon at nucleotide 3417. Two non-structural proteins are expressed from this ORF. The first is a 126 kDa protein (130K) containing the nucleotide binding and putative helicase activities. The second is a 183 kDa protein (180K), which is a translational readthrough of the amber stop codon in about 5-10% of the translational events. The 183 kDa protein contains the functional domains of the 126 kDa protein and a novel domain with homology to RNA-dependent RNA polymerases. At least two subgenomic mRNAs with a common 3′ terminus are also produced after TMV infection. These encode a 30 kDa movement protein and a 17.5 kDa coat protein. The 3′ terminus of TMV genomic RNA can be folded into a series of pseudoknots followed by a tRNA-like structure. The genomic RNA cannot function as a messenger for the synthesis of coat protein. Other genes are expressed during infection by the formation of monocistronic, 3′-coterminal subgenomic mRNAs, including one (LMC) encoding the 17.5K coat protein and another (I₂) encoding a 30K protein. The 30K protein has been detected in infected protoplasts as described in (Miller, 1984), and it is involved in the cell-to-cell transport of the virus in an infected plant as described by (Deom, 1987). The functions of the two large proteins are unknown, however, they are thought to function in RNA replication and transcription.

Several ds RNA molecules, including ds RNAs corresponding to the genomic, 12 and LMC RNAs, have been detected in plant tissues infected with TMV. These RNA molecules are presumably intermediates in genome replication and/or mRNA synthesis processes, which appear to occur by different mechanisms.

TMV assembly apparently occurs in plant cell cytoplasm, although it has been suggested that some TMV assembly may occur in chloroplasts since transcripts of ctDNA have been detected in purified TMV virions. Initiation of TMV assembly occurs by interaction between ring-shaped aggregates (“discs”) of coat protein (each disc consisting of two layers of 17 subunits) and a unique internal nucleation site in the RNA; a hairpin region about 900 nucleotides from the 3′-end in the common strain of TMV. Any RNA, including subgenomic RNAs containing this site, may be packaged into virions. The discs apparently assume a helical form on interaction with the RNA, and assembly (elongation) then proceeds in both directions (but much more rapidly in the 3′- to 5′-direction from the nucleation site).

Another member of the Tobamoviruses, the Cucumber Green Mottle Mosaic virus watermelon strain (“CGMMV-W”) is related to the cucumber virus (Nozu et al., 1971). The coat protein of CGMMV-W interacts with RNA of both TMV and CGMMV to assemble viral particles in vitro (Kurisu et al., 1976).

Several strains of the tobamovirus group are divided into two subgroups, on the basis of the location of the origin of assembly. Subgroup I, which includes the vulgare, OM, and tomato strain, has an origin of assembly about 800-1000 nucleotides from the 3′-end of the RNA genome, and outside the coat protein cistron (Lebeurier, Nicolaieff, and Richards, 1977); and (Fukuda, 1980). Subgroup II, which includes CGMMV-W and cowpea strain (Cc) has an origin of assembly about 300-500 nucleotides from the 3′-end of the RNA genome and within the coat protein cistron. The coat protein cistron of CGMMV-W is located at nucleotides 176-661 from the 3′-end. The 3′ noncoding region is 175 nucleotides long. The origin of assembly is positioned within the coat protein cistron (Meshi, 1983).

### Brome Mosaic Virus Group

BMV is a member of a group of tripartite, ss, RNA-containing plant viruses commonly referred to as the bromoviruses. Each member of the bromoviruses infects a narrow range of plants. Mechanical transmission of bromoviruses occurs readily, and some members are transmitted by beetles. In addition to BMV, other bromoviruses include broad bean mottle virus and cowpea chlorotic mottle virus.

Typically, a bromovirus virion is icosahedral, with a diameter of about 26 μm, containing a single species of coat protein. The bromovirus genome has three molecules of linear, positive-sense, ss RNA, and the coat protein mRNA is also encapsidated. The RNAs each have a capped 5′-end, and a tRNA-like structure (which accepts tyrosine) at the 3′-end. Virus assembly occurs in the cytoplasm. The complete nucleotide sequence of BMV has been identified and characterized as described by (Ahlquist, Luckow, and Kaesberg, 1981).

### Hordeivirus Group

Hordeiviruses are a group of ss, positive sense RNA-containing plant viruses with three or four part genomes. Hordeiviruses have rigid, rod-shaped virions. Hordeivirus is composed of four members: BSMV, poa semilatent virus (“PSLV”), lychnis ringspot virus (“LRSV”), and anthoxanthum latent blanching virus (“ALBV”) (Jackson, et al., 1989). BSMV is the type member of this group of viruses. BSMV infects a large number of monocot and dicot species including barley, oat, wheat, corn, rice, spinach, and N. benthamiana. Local lesion hosts include Chenopodium amaranticolor, and Nicotiana tabacum cv. Samsun. BSMV is not vector transmitted but is mechanically transmissible and in some hosts, such as barley, is also transmitted through pollen and seed. Most strains of BSMV have three genomic RNAs referred to as RNAα (or αRNA), RNAβ (or βRNA), and RNAγ (or γRNA). At least one strain, the Argentina mild (AM) strain has a fourth genomic RNA that is essentially a deletion mutant of the RNAγ. All genomic RNAs are capped at the 5′ end and have tRNA-like structures at the 3′ end. Virus replication and assembly occurs in the cytoplasm. The complete nucleotide sequence of several strains of BSMV has been identified and characterized (reviewed by Jackson, et al., 1989), and infectious cDNA clones are available (Petty et al., 1989).

BSMV is a plus-sense ss RNA virus that is able to infect plants of the Chenopodiaceae, Gramineae, and Solanaceae families, including, but not limited to, the following species: Anthoxanthum aristatum, Anthoxanthum odoratum, Avena sativa, Beta vulgaris, Bromus secalinus, Bromus tectorum, Chenopodium album, Chenopodium amaranticolor, Chenopodium quinoa, Dactylis glomerata, Echinochloa crus-galli, Elytrigia intermedia, Eragrostis cilianensis, Festuca pratensis, Hordeum vulgare, Lagurus ovatus, Lolium multiflorum, Lolium perenne, Lolium persicum, Lolium temulentum, Lophopyrum elongatum, Nicotiana tabacum, Oryza sativa, Oryzopsis miliacea, Panicum capillare, Panicum miliaceum, Phalaris arundinacea, Phalaris paradoxa, Phleum arenarium, Phleum pratense, Poa annua, Poa pratensis, Secale cereale, Setaria italica, Setaria macrostachya, Setaria viridis, Sorghum bicolor, Spinacia oleracea, Triticum aestivum, Triticum durum, and Zea mays. The method of transmission does not involve a vector and is by mechanical inoculation by seed (up to 90-100%) and by pollen to the pollinated plant. BSMV virions are rod-shaped, not enveloped, and usually straight. (Brunt et al., Plant Viruses Online: Descriptions and Lists from the VIDE Database, URL http://biology.anu.edu.au/Grouops/MES/vide/, 1996 onwards).

The BSMV virion contains 3.8-4% nucleic acid, 96% protein, and 0% lipid by weight. The BSMV genome consists of three ss linear RNA (designated RNAα, RNAβ, and RNAγ). The total genome size is 10.289 kb (Brunt et al., 1996). Each genomic RNA has a 7-methylguanosine cap at its 5′ terminus and contains the initial sequence m⁷ GpppGUA, and has a highly conserved 3′ terminus that has a polyadenylate (poly A) sequence that separates the coding region of each RNA from a 238 nucleotide 3′ terminal tRNA-like structure that can be aminoacylated with tyrosine. BSMV encodes a total of seven polypeptides. RNAα encodes αa, a 130 kDa protein which is believed to be an integral component of viral replicase. αa has a putative methyltransferase domain near the N-terminus and a nucleotide binding motif near the C-terminus (Jackson et al., 1991). When αa of BSMV strain N18 (non-pathogenic to oat) had more than half of its ORF replaced with the homologous αa of BSMV strain CV42 (pathogenic to oat), the gene homologous gene replacement enabled strain N18 to infect oat. In addition, a single amino acid substitution or up to six single amino acid substitutions (including the substitution of two adjacent amino acids) in αa of strain N18 enabled strain N18 to infect oat (Weiland and Edwards, 1996). RNAβ encodes four polypeptides: βa, the 22 kDa coat protein; βb, a 60 kDa disease-specific protein, which contains a nucleotide binding motif similar to αa; βc, a 17 kDa protein of unknown function but which is required for infectivity in barley (N. benthamiana and C. amaranticolor); and, βd, a 14 kDa protein essential for systemic infection and associated with the membrane fraction of infected barley. The ORFs of βb, βc and βd are tightly organized to form a triple gene block (“TGB”) whereby βd overlaps βb and βc. The TGB is similar in organization to the overlapping gene blocks found in furoviruses, potexviruses, and potato virus M, a carlavirus (Jackson et al., 1991). RNAγ encodes two ORFs: γa and γb. The γa ORF encodes a second replicase component, γa, that contains the GDD polymerase motif that is universally present in the replicases of plus-sense RNA viruses. The γb ORF encodes a 17 kDa cysteine rich protein, γb, contains a cysteine-rich region. BSMV with mutations that introduce single or up to four single amino acid substitutions in γb, when used to inoculate barley plants, resulted in altered symptom phenotype (Donald and Jackson, 1994). BSMV is of interest to provide new and improved vectors for the genetic manipulation of plants.

### Rice Necrosis Virus

RNV is a member of the Potato Virus Y Group or Potyviruses. The Rice Necrosis virion is a flexuous filament comprising one type of coat protein (molecular weight about 32,000 to about 36,000) and one molecule of linear positive-sense ss RNA. The Rice Necrosis virus is transmitted by Polymyxa oraminis (a eukaryotic intracellular parasite found in plants, algae and fungi).

### Geminiviruses

Geminiviruses are a group of small, ss DNA-containing plant viruses with virions of unique morphology. Each virion consists of a pair of isometric particles (incomplete icosahedral), composed of a single type of protein (with a molecular weight of about 2.7-3.4×10⁴). Each geminivirus virion contains one molecule of circular, positive-sense, ss DNA. In some geminiviruses (i.e., Cassaya latent virus and bean golden mosaic virus) the genome appears to be bipartite, containing two ss DNA molecules.

### Potyviruses

Potyviruses are a group of plant viruses, which produce polyprotein. A particularly preferred potyvirus is tobacco etch virus (“TEV”). TEV is a well characterized potyvirus and contains a positive-strand RNA genome of 9.5 kilobases encoding for a single, large polyprotein that is processed by three virus-specific proteinases. The nuclear inclusion protein “a” proteinase is involved in the maturation of several replication-associated proteins and capsid protein. The helper component-proteinase (HC-Pro) and 35-kDa proteinase both catalyze cleavage only at their respective C-termini. The proteolytic domain in each of these proteins is located near the C-terminus. The 35-kDa proteinase and HC-Pro derive from the N-terminal region of the TEV polyprotein.

The selection of the genetic backbone for the viral vectors of the instant invention may depend on the plant host used. The plant host may be a monocotyledonous or dicotyledonous plant, plant tissue, plant organ, or plant cell. Typically, plants of commercial interest, such as food crops, seed crops, oil crops, ornamental crops and forestry crops are preferred. For example, wheat, rice, corn, potato, barley, tobacco, soybean canola, maize, oilseed rape, lilies, grasses, orchids, irises, onions, palms, tomato, the legumes, or Arabidopsis, can be used as a plant host. Host plants may also include those readily infected by an infectious virus, such as Nicotiana, preferably, Nicotiana benthamiana, or Nicotiana clevelandii.

The source of the protein of interest and nucleic acid encoding the protein of interest can be derived or obtained from one or more donor organisms. The donor organism may be any organism of any classification, which includes Kingdom Monera, Kingdom Protista, Kingdom Fungi, Kingdom Plantae and Kingdom Animalia. Kingdom Monera includes subkingdom Archaebacteriobionta (archaebacteria): division Archaebacteriophyta (methane, salt and sulfolobus bacteria); subkingdom Eubacteriobionta (true bacteria): division Eubacteriophyta; subkingdom Viroids; and subkingdom Viruses. Kingdom Protista includes subkingdom Phycobionta: division Xanthophyta (yellow-green algae), division Chrysophyta (golden-brown algae), division Dinophyta (Pyrrhophyta) (dinoflagellates), division Bacillariophyta (diatoms), division Cryptophyta (cryptophytes), division Haptophyta (haptonema organisms), division Euglenophyta (euglenoids), division Chlorophyta, class Chlorophyceae (green algae), class Charophyceae (stoneworts), division Phaeophyta (brown algae), and division Rhodophyta (red algae); subkingdom Mastigobionta: division Chytridiomycota (chytrids), and division Oomycota (water molds); subkingdom Myxobionta: division Acrasiomycota (cellular slime molds), and division Myxomycota (true slime molds). Kingdom Fungi includes division Zygomycota (coenocytic fingi): subdivision Zygomycotina; and division Eumycota (septate fungi): subdivision Ascomycotina 000 (cup fungi), subdivision Basidiomycotina (club fungi), subdivision Deuteromycotina (imperfect fungi), and subdivision Lichenes. Kingdom Plantae includes division Bryophyta, Hepatophyta, Anthocerophyta, Psilophyta, Lycophyta, Sphenophyta, Pterophyta, Coniferophyta, Cycadeophyta, Ginkgophyta, Gnetophyta and Anthophyta. Kingdom Animalia includes: Porifera (Sponges), Cnidaria (Jellyfishes), Ctenophora (Comb Jellies), Platyhelminthes (Flatworms), Nemertea (Proboscis Worms), Rotifera (Rotifers), Nematoda (Roundworms), Mollusca (Snails, Clams, Squid & Octopus), Onychophora (Velvet Worms), Annelida (Segmented Worms), Arthropoda (Spiders & Insects), Phoronida, Bryozoa (Bryozoans), Brachiopoda (Lamp Shells), Echinodermata (Sea Urchins & starfish), and Chordata (Vertebrata-Fish, Birds, Reptiles, Mammals). A preferred donor organism is human. The donor organism may be any virus.

There can be one or more polynucleotides. The protein of interest may be encoded and expressed from one or a first polynucleotide and the genetic element capable of reducing a protease activity may be on another or second polynucleotide.

The protein of interest may be present in only certain tissue(s) or region(s) of the host. As such, the responsible agent of reducing protease activity should be active in the same tissue(s) or region(s). Tissue specific expression is known in a number of host parts such as plant seeds (Batchelor et al., 2000; Tanaka et al., 2001; Yamagata et al., 2000), leaves (Jorda et al., 1999; Meichtry, Amrhein, and Schaller, 1999), roots (Jorda, Conejero, and Vera, 2000; Meichtry, Amrhein, and Schaller, 1999) and as mentioned above.

Expression of a plant subtilisin-like protease has also been proposed in the regulation of stomatal distribution and density in Arabidopsis thaliana (Berger and Altmann, 2000a; Berger and Altmann, 2000b). Thus, reducing its activity for such purposes is also a use for the present invention.

The viral vector can also be a monopartite tobravirus RNA-1 comprising an inserted foreign RNA sequence operably linked to the 3′-end of the stop codon of the RNA sequence that codes for a 16 kDa cysteine-rich protein of RNA-1. The host can be any cell capable of expressing the protein of interest and/or the genetic element capable of reducing a protease activity. The host can be any plant cell. The plant cell may a protoplast, a recombinant cell, a transgenic cell, a non-transgenic cell, or a cell that is part of a cell culture, cell tissue, plant organ, or an entire plant organism. A protoplast is a plant cell that has the cell wall removed. The plant cell can be a dicot or a monocot plant cell. Preferably, the plant cell is a dicot plant cell. More preferably, the dicot plant cell is a Nicotiana benthamiana cell. The host may be of a species or strain that can be infected with a viral genome or a recombinant virus obtained from a virus that can infect the host.

Plant hosts include plants of commercial interest, such as food crops, seed crops, oil crops, ornamental crops and forestry crops. For example, wheat, rice, corn, potatoes, barley, tobaccos, soybean canola, maize, oilseed rape, Nicotiana sp. can be selected as a host plant. Plants without commercial interest may also be used, for example, Arabidopsis sp. In particular, host plants capable of being infected by a virus containing a recombinant viral nucleic acid are preferred. Preferred host plants include Nicotiana. More preferably, the host plants are N. benthamiana, N. excelsiana, N. clevelandii or tobacco.

Individual clones may be transfected into the plant host, such as (1) protoplasts; (2) cell or tissue cultures, (3) whole plants; or (4) plant tissues, such as leaves of plants (Dijkstra, 1998; Foster and Taylor, 1998). In some embodiments of the instant invention, the delivery of the recombinant plant nucleic acid into the plant may be affected by the inoculation of in vitro transcribed RNA, inoculation of virions, or internal inoculation of plant cells from nuclear cDNA, or the systemic infection resulting from any of these procedures. In all cases, the co-infection may lead to a rapid and pervasive systemic expression of the desired nucleic acid sequences in plant cells.

The host can be infected with a recombinant viral nucleic acid or a recombinant plant virus by conventional techniques. Suitable techniques include, but are not limited to, leaf abrasion, abrasion in solution, high velocity water spray, and other injury of a host as well as imbibing host seeds with water containing the recombinant viral RNA or recombinant plant virus. More specifically, suitable techniques include:

(a) Hand Inoculations. Hand inoculations are performed using a neutral pH, low molarity phosphate buffer, with the addition of a particulate such as celite or carborundum (usually about 1%). One to four drops of the preparation is put onto the upper surface of a leaf and gently rubbed.

(b) Mechanized Inoculations of Plant Beds. Plant bed inoculations are performed by spraying (gas-propelled) the vector solution into a tractor-driven mower while cutting the leaves. Alternatively, the plant bed is mowed and the vector solution sprayed immediately onto the cut leaves.

(c) High Pressure Spray of Single Leaves. Single plant inoculations can also be performed by spraying the leaves with a narrow, directed spray (50 psi, 6-12 inches from the leaf) containing approximately 1% carborundum in the buffered vector solution.

(d) Vacuum Infiltration. Inoculations may be accomplished by subjecting a host organism to a substantially vacuum pressure environment in order to facilitate infection.

(e) High Speed Robotics Inoculation. Especially applicable when the organism is a plant, individual organisms may be grown in mass array such as in microtiter plates. Machinery such as robotics may then be used to transfer the nucleic acid of interest.

(f) Ballistics (High Pressure Gun) Inoculation. Single plant inoculations can also be performed by particle bombardment. A ballistics particle delivery system (BioRad Laboratories, Hercules, (A) can be used to transfect plants such as N. benthamiana as described previously (Nagar et al., 1995).

An alternative method for introducing recombinant viral nucleic acids into a plant host is a technique known as agroinfection or Agrobacterium-mediated transformation (also known as Agro-infection) as described by (Grimsley, 1987). This technique makes use of a common feature of Agrobacterium, which colonizes plants by transferring a portion of their DNA (the T-DNA) into a host cell, where it becomes integrated into nuclear DNA. The T-DNA is defined by border sequences that are 25 base pairs long, and any DNA between these border sequences is transferred to the plant cells as well. The insertion of a recombinant plant viral nucleic acid between the T-DNA border sequences results in transfer of the recombinant plant viral nucleic acid to the plant cells, where the recombinant plant viral nucleic acid is replicated, and then spreads systemically through the plant. Agro-infection has been accomplished with potato spindle tuber viroid (PSTV) (Gardner, 1986); CaV (Grimsley, 1986); MSV (Grimsley, 1987), and (Lazarowitz, 1988)) digitaria streak virus (Donson et al., 1988), wheat dwarf virus (Hayes, 1988) and tomato golden mosaic virus (TGMV) (Elmer, 1988) and (Gardiner, 1988). Therefore, agro-infection of a susceptible plant could be accomplished with a virion containing a recombinant plant viral nucleic acid based on the nucleotide sequence of any of the above viruses. Particle bombardment or electroporation or any other methods known in the art may also be used.

In some embodiments of the instant invention, infection may also be attained by placing a selected nucleic acid sequence into an organism such as E. coli, or yeast, either integrated into the genome of such organism or not, and then applying the organism to the surface of the host organism. Such a mechanism may thereby produce secondary transfer of the selected nucleic acid sequence into a host organism. This is a particularly practical embodiment when the host organism is a plant. Likewise, infection may be attained by first packaging a selected nucleic acid sequence in a pseudovirus. Such a method is described in U.S. Pat. No. 5,443,969 (Wilson and Hwang-Lee, 1995). Though the teachings of this reference may be specific for bacteria, those of ordinary skill in the art will readily appreciate that the same procedures could easily be adapted to other organisms.

Plant may be grown from seed in a mixture of “Peat-Lite Mix™” (Speedling, Inc. Sun City, Fla.) and Nutricote™ controlled release fertilizer 14-14-14 (Chiss-Asahi Fertilizer Co., Tokyo, Japan). Plants may be grown in a controlled environment provided 16 hours of light and 8 hours of darkness. Sylvania “Gro-Lux/Aquarium” wide spectrum 40 watt fluorescent grow lights (Osram Sylvania Products, Inc. Danvers, Mass.) may be used. Temperatures may be kept at around 27° C. during light hours and 21° C. during dark hours. Humidity may be between 60 and 85%.

In the examples below, we describe the purification and characterization of novel Nicotiana benthamiana plant proteases that are involved in the cleavage of a mammalian therapeutic protein in vitro and in vivo. Based on molecular, biochemical and functional properties, these enzymes are classified as subtilisin-like serine proteases and named Nicotianalisins. In addition, we describe a method used to clone two members of the N. benthamiana subtilisin-like protease gene family. This method utilizes the parameters of strict conservation of the catalytic triad domain in this family of proteases and can be used to clone similar proteases from other species of plants. These examples also describe the isolation of thirteen other members of the N. benthamiana subtilisin-like protease gene family from a sequenced and annotated N. benthamiana library using databases searching tools. Molecular approaches to down regulating the activity of this protease activity in vivo as a means to produce human therapeutics in plants are also described.

Cloning of the Nicotianalisin genes allowed the development of methods to reduce Nicotianalisin protease activity in order to decrease the proteolysis of recombinant proteins expressed in the plant. Expression of the cloned Nicotianalisin gene may be performed to produce even higher amounts of the protease, which may be purified and used as a product per se in purified or essentially isolated form.

When the protease-labile protein of interest is native to the host cell, one need only inhibit the proteases in order to increase effective recovery of the protein of interest. The present invention using a vector with the genetic element may be used to increase the recovery of that protein of interest. This and similar methods are particularly effective when recovering proteins from the interstitial fluid which contains endogenous protease. A number of protease inhibitors are known to be secreted extracellularly (Horisberger et al, Histochemistry. 1983;77(3):313-21) and such are preferred.

The following examples further illustrate the present invention. While the examples show reducing one protease activity, the same techniques may simultaneously reduce plural protease activities; for example, by using a non-specific protease inhibitor or plural genetic elements, each specific for different protease activities. These examples are intended merely to be illustrative of the present invention and are not to be construed as being limiting.

Throughout the specification, the emphasis has been on reducing protease activity. However, other biological activities are present inside a cell, which may degrade the protein of interest. These include other hydrolases acting on ester, amide or glucosidic, cyclic amides (e.g. beta-lactamase), isomerases, asparginases, saccharidase, nuclease and small organic molecule cleaving enzymes. For example, a saccharidase may alter (or even completely remove) the glycosylation pattern of the protein of interest. Inhibition of these enzymes are also contemplated as part of the present invention.

### EXAMPLES

### Example 1

Purification and Biochemical Characterization of Nicotiana benthamiana Subtilisin-Like Serine Protease.

Initial Characterization of Protease Activity

When a plant viral vector expression system (Kumagai et al., 1995; McCormick et al., 1999) was utilized to express the secreted, mammalian recombinant protein, hGH, significant degradation of the target protein was observed. Experiments were performed to identify the proteolytic activity responsible for the degradation. N. benthamiana plants were grown in a controlled environment with 27° C. day and 23° C. night temperatures, a 12 hour photoperiod, and 86% relative humidity. Plants were inoculated three weeks post sow date with infectious transcripts of a plant viral vector comprising an hGH gene sequence in-frame with a tobacco extensin signal peptide as previously described for other tobamovirus expression studies (Kumagai et al., 1995; McCormick et al., 1999). Eight to ten days post-inoculation (dpi), virally-infected plant material was harvested and used for isolation of the plant interstitial fluid (IF) as previously described (McCormick et al., 1999).

Aliquots of the plant IF fraction were separated by SDS-PAGE and levels of hGH protein were detected by immunoblot analysis using anti-hGH polyclonal antibody (Sigma, St. Louis, Mo.). Briefly, proteins were separated on precast gels with an Xcell II Mini-Cell apparatus (Invitrogen, Carlsbad, Calif.) in the buffer system of Laemmli (Laemmli, 1970). Proteins were electrophoretically transferred (1 hr, 100 volts, 4° C.) to a nitrocellulose membrane (0.45 μm)(Schleicher and Schuell, Dassel, Germany). After blocking of nonspecific binding sites with 5% non-fat dried milk in Tween 20/Tris-HCl buffered Saline (TBST) for 2 hr, the blot was incubated for 2 hr with a 1:1000 dilution of anti-hGH antibody. Blots were developed using goat-anti-rabbit alkaline phosphatase-conjugated secondary antibodies (Sigma) as per manufacturer's instructions.

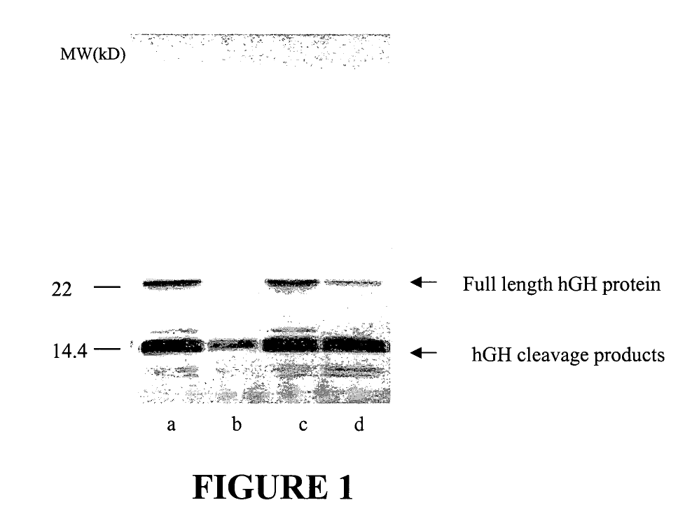

FIG. 1 reveals the inhibitory effects observed using specific protease inhibitors against the plant protease. At zero time, prior to the addition of any inhibitors, both full length and hGH cleavage products were detected (FIG. 1). When chymostatin, a specific inhibitor of chymotrypsin- and subtilisin- like proteases (Umezawa, 1976), was added to the IF extract, the inhibition of further degradation of the intact hGH protein was observed. The addition of potato protease inhibitor I (PI-I), a specific inhibitor of chymotrypsin-like serine protease activity (Plunkett et al., 1982), also inhibited degradation of the recombinant protein, but not as well as chymostatin. In the absence of any protease inhibitor, the full-length hGH protein was completely degraded in the plant IF extract.

Plant Protease Inhibitor Studies

To further classify the protease activity in the plant IF extract, protease inhibitor studies were performed. Standard inhibitors from different classes of proteases were used. An aliquot of the plant IF was incubated with each inhibitor for 30 min at 24° C., and the free enzyme was incubated under the same conditions without the inhibitor to serve as the control. The protease activity after the inhibition was measured and compared to the control. The results of this study, and the specifications of each inhibitor (Twining, 1984), are summarized in Table 1. Protease inhibitors that specifically target the class of serine proteases exhibited 100% inhibition of protease activity in an in vitro assay. Interestingly, approximately 40% inhibition was observed when inhibitors of either chymotrypsin-like proteases or elastases were used.

TABLE 1
Nicotianalisin inhibition in the presence of inhibitors from
different classes of proteases.
IF was prepared from viral vector-infected plants and incubated
with each inhibitor for 30 min at 24° C. Protease activity
following inhibitor treatment was measured using 0.3 mM
N-Suc-AAPL-p-NA (see below). Reduction of the protease
activity was reported as percent inhibition compared to
the activity in the IF from an uninfected plant (control).

| | | % | | |
|---|---|---|---|---|
| No. | Inhibitor | Inhibition | Class | Comments |
| 1 | Control | 0 | Serine/Irr* | |
| 2 | 3-4 DCI | 100 | Serine/Irr | Fast inhibitor |
| 3 | PMSF | 100 | Serine/Irr | |
| 4 | Chymostatin | 100 | Serine/Irr | |
| 5 | TLCK | 0 | Serine/Irr | Trypsin-like |
| 6 | TPCK | 38 | Serine/Irr | Chymotrypsin-like |
| 7 | N-CBZ-GGF-CK | 70 | Serine/Irr | Active site titrant |
| 8 | pI-I | 30 | Serine | Chymotrypsin-like |
| 9 | pI-II | 2 | Serine | Trypsin-like |
| 10 | Trypsin-Chymo I | 55 | Serine | Trypsin/ |
| | | | | chymotrypsin |
| 11 | Aprotinin | 46 | Serine/Rev* | |
| 12 | Elastinal | 41 | Serine/Rev | Elastase-like |
| 13 | Antipain | 77 | Serine/ | Trypsin and |
| | | | Cysteine | many cysteine-like |
| 14 | Leupeptin | 18 | Ser/Cysteine | Trypsin and |
| | | | | many cysteine-like |
| 15 | E-64 | 5 | Cysteine/Irr | Active site titrant |
| 16 | Cystain | 2 | Cysteine/Rev | |
| 17 | EDTA | 5 | Metallo/Rev | Chelator |
| 18 | Amastatin | 5 | Metallo/Rev | Aminopeptidase I |
| 19 | 1-10 Phenantrolin | 11 | Metallo/Rev | Chelator |
| 20 | Pepstatin A | 6 | Aspartic/Rev | |

*Irr—irreversible protease inhibitor;
Rev—reversible protease inhibitor

No. 7 has SEQ ID NO:

A large number of other protease inhibitors may be used. Particularly preferred are polypeptide protease inhibitors, which may be co-expressed with the protein of interest. For example, apolipoprotein A-I, tissue inhibitors of matrix metalloproteinases (MMPs), serine protease inhibitor, soybean trypsin inhibitor, soybean and other cysteine protease inhibitor, soyacystatin N (scN), winged bean chymotrypsin inhibitor, tomato protease inhibitor, etc.

Plant Protease Enzyme Assays

To further characterize the plant proteolytic activity, experiments were initiated to partially purify the plant protease. In order to purify the protease, enzyme assays were developed and utilized. Proteolytic activity was monitored by the hydrolysis of a synthetic substrate, hydrolysis of a protein substrate and/or by an in situ gel procedure. Assays based on synthetic substrates were performed in 100 mM Tris-HCl, pH 7.0, 5 mM CaCl₂ for 30 min at 37° C. The synthetic substrate was N-Succinyl-Alanine-Alanine-Proline-Leucine-para-nitroanilide (N-Suc-Ala-Ala-Pro-Leu-p-NA SEQ ID NO:) and/or N-Succinyl-Alanine-Alanine-Proline-Phenylalanine-para-nitroanilide (N-Suc-Ala-Ala-Pro-Phe-p-NA SEQ ID NO:) at 0.3 mM final concentration (Largman et al., 1980; Siekierka et al., 1989). The absorbance of the p-nitroaniline (p-NA) produced was measured at 410 nm (Erlanger, Kokowsky, and Cohen, 1961; Nakajima et al., 1979). The activity unit was defined as the amount of the enzyme capable of producing 1 nanomole of p-NA per min under the conditions of the experiment with an absorption coefficient of 8.8 mM cm⁻¹ (Erlanger, Kokowsky, and Cohen, 1961; Nakajima et al., 1979). The rate of hydrolysis of N-Succinyl-Ala-Ala-Pro-Phe-thiobenzylester SEQ ID NO: in 10% DMSO and 4,4′-dithiodipyridine was monitored at 324 nM using an extinction coefficient of 19800 cm⁻¹ M⁻¹ (Barrett and Kirschke, 1981). General proteolytic activity was measured with 2.0% azocasein or 2.0% azoalbumin and Fluorescein Isothiocyanate Casein (FITC-casein; (Twining, 1984)). The reaction was started by addition of 10 μl of protease solution to 70 μl of reaction mixture (100 mM buffer Tris-HCl pH 7.0 containing 0.04% FTC-casein, 0.5 mM DDT, 2 mM CaCl₂) and incubation for 1 hr at 37° C. The reaction was stopped by addition of 80 μl of 10% TCA and incubation for 15 min at −20° C. The mixture was centrifuged for 10 min at 10,000×g at 4° C. 10 μl of the supernatant solution was added to 150 μl Tris-HCl (0.5 M, pH 8.5), and fluorescence was measured at an excitation wavelength of 490 nm and an emission wavelength of 525 nm using a fluorescence plate reader ((Erlanger, Kokowsky, and Cohen, 1961; Nakajima et al., 1979), Molecular Devices).

The in situ protease gel assay was carried out in pre-cast 10% Zymogram gels (Invitrogen, Carlsbad, Calif.) as per manufacturer's protocols.

Partial Purification and Further Characterization of the Plant Protease

Following the initial observation that the plant protease activity was inhibited by both chymostatin and potato PI-I (FIG. 1), experiments were initiated to partially purify the protease from N. benthamiana leaves. Plants were grown and IF extracts were prepared as described above. The IF was filtered through a 0.8μ Sartorius GF membrane to remove most of the Ribulose-1,5-bisphosphate carboxylase/oxygenase (Rubisco) and green pigments. The diafiltered extract was separated further by SP sepharose (Pharmacia) ion exchange chromatography at pH 5.2. Proteolytic activity from column chromatography fractions was monitored by the hydrolysis of the synthetic substrate, N-Suc-Ala-Ala-Pro-Leu-p-NA SEQ ID NO:, as described above.

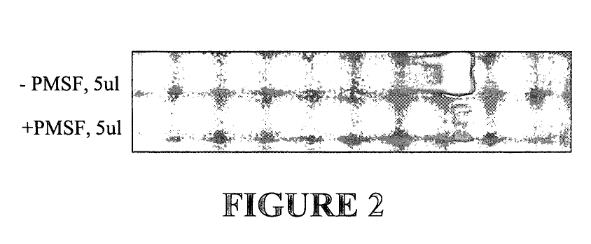

When the partially purified fraction containing highest proteolytic activity was treated with or without phenylmethanesulfonyl fluoride (PMSF) (Sigma), a general serine protease inhibitor (Moss and Fahrney, 1978), a significant reduction of protease activity was observed (FIG. 2). Using the in situ gel protease assay referenced above, a significant inhibition of clearing was observed in the Zymogram gelatin gel. This observation indicates that PMSF is a potent inhibitor of the partially purified plant protease, and therefore, the plant protease belongs to the serine protease family.

Purification of the Nicotianalisin Enzyme

In order to fully characterize the plant protease, Nicotianalisin, it was necessary to purify the enzyme. N. benthamiana plants were grown and IF extracts were prepared and diafiltered as described above. Proteolytic activity from column chromatography fractions was monitored by the hydrolysis of synthetic substrates, as described above.

Clarified IF was concentrated by an ultrafiltration system, using a 10,000 MWCO Amicon spiral membrane (Millipore). The supernatant solution of a 30% ammonium sulfate cut was filtered through a ≧0.2≦0.65μ filter and applied to a Butyl Sepharose (Pharmacia) chromatography column equilibrated with 30% saturated ammonium sulfate in 25 mM imidazole buffer, pH 6.0 (buffer A). Unbound proteins were washed from the column with buffer A, and Nicotianalisin activity was eluted with a linear gradient of decreasing ammonium sulfate in buffer A. Fractions with protease activity were pooled and dialyzed overnight against 25 mM Tris-HCl, pH 7.0 or concentrated and diafiltrated with 25 mM Tris-HCl, pH 7.0 using an ultrafiltration system. The concentrated active pool from Butyl Sepharose was applied to a DEAE Sepharose column equilibrated with 25 mM Tris-HCl, pH 7.0. After washing the unbound proteins from the column, the activity was eluted with a linear NaCl gradient from 0 to 200 mM in the equilibration buffer. Active fractions were collected and concentrated using a BioMax 10,000 MWCO membrane (Millipore), and applied to a Sephacryl S-100 gel filtration column (Pharmacia). The protein was eluted using 50 mM Tris-HCl buffer, pH 7.0, containing 150 mM NaCl. The pooled S-100 fractions were purified further using a Superose-12 (Pharmacia) size exclusion column equilibrated in 50 mM Tris-HCl buffer, pH 7.0, containing 150 mM NaCl. The protein concentration was measured using Bradford protein reagent (Bio-Rad Laboratories, Hercules, Calif.).

The steps in the purification of Nicotianalisin from the IF of N. benthamiana leaves are summarized in Table 2. Protease activity was retained nearly quantitatively and the activity eluted as a single peak during all column purification steps. Nicotianalisin was purified 306-fold with a specific activity of 3171 units/mg total protein after the final purification step.

TABLE 2
Purification of Nicotianalisin from N. benthamiana leaf.

| | Total | Total | Specific | | Purification |
|---|---|---|---|---|---|
| Purification | Activity¹ | Protein | Activity | Yield | factor |
| Steps | units | mg | units/mg | percent | fold |
| IF | 3306 | 319 | 10.4 | 100.0 | 1 |
| UF-DF | 3046 | 232 | 13.1 | 92.1 | 1 |
| Butyl | 2087 | 25 | 83.5 | 63.1 | 8 |
| Sepharose | | | | | |
| DEAE | 771 | 2.2 | 350.4 | 23.3 | 34 |
| Sepharose | | | | | |
| Sephacryl | 414 | 0.35 | 1181.5 | 12.5 | 114 |
| S-100 | | | | | |
| Superose-12 | 203 | 0.064 | 3170.9 | 6.1 | 306 |

¹Based on enzyme activity using Suc-AAPL-pNA SEQ ID NO:, where one unit is 1 nmol pNA released per min.
Molar absorption coefficient 8.8 mM⁻¹ cm⁻¹

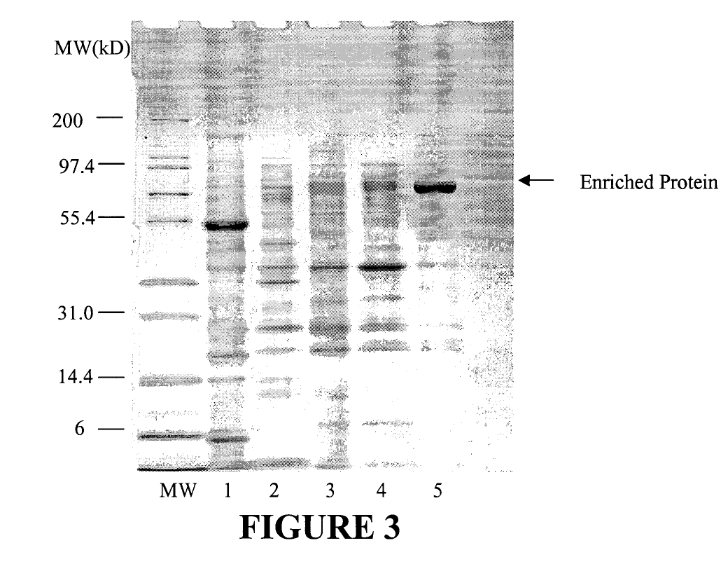

FIG. 3 represents an overview of the purification steps analyzed by 14% SDS-PAGE stained with Coomassie brilliant blue. Protein samples of the various purification steps (2.5 μg total protein) were loaded per lane. Enrichment of a protein band with an apparent molecular mass of 80 kD was observed after the final purification step (FIG. 3).

The protease was also purified using other combinations of column chromatography, such as, SP-Sepharose, isoelectric focusing, and Mono-Q HR. The active fractions from the DEAE column (see table above) were pooled and applied to an isoelectric focusing column. The activity was eluted over a pH range of 7 to 3.5 using polybuffer 74 (Amersham Pharmacia Biotech). The majority of the Nicotianalisin activity was eluted at acidic pH. Different isozymes were also separated using cation exchange chromatography (SP-Sepharose, Mono-S) by loading the column over a pH range of 5.2 to 4.5 and eluting with a linear gradient of 0 to 250 mM NaCl in binding buffer.

Biochemical Characterization of Purified Nicotianalisin

Following purification of the plant protease, various biochemical analyses were performed in order to fully characterize the enzyme.

Nicotianalisin Amino acid Sequence Homologous to Other Plant Subtilisins:

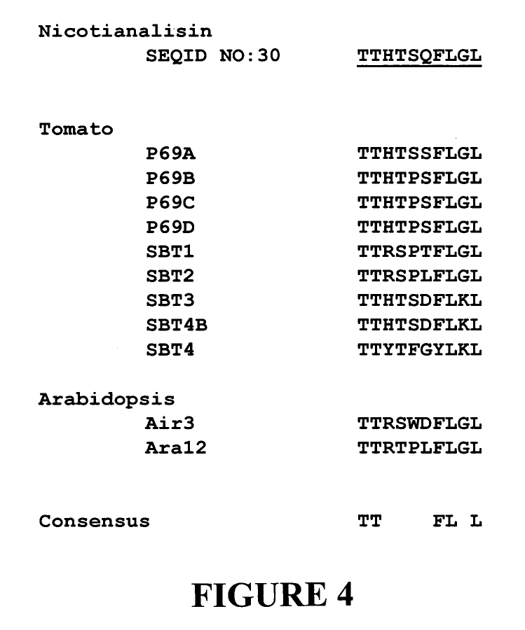

N-terminal amino acid sequence. An aliquot of the purified Nicotianalisin protein was immobilized on PVDF (ProBlot, Applied Biosystems) membrane and the N-terminal sequence of the mature protein was determined using an Applied Biosystems Sequencer. The N-terminal amino acid sequence of the purified, mature N. benthamiana secreted protease was determined to be TTHTSQFLGL (SEQ ID NO: 30) (FIG. 4). The site of propeptide processing appears to occur amino-terminal to a pair of threonine residues. This sequence is homologous to other plant subtilisin-like proteases (FIG. 4) and contains the conserved motif that has been described in other plant subtilisin-like proteases (Meichtry, Amrhein, and Schaller, 1999).

Internal amino acid peptide sequence. The amino acid sequence of an internal fragment of the purified protease was determined using electrospray ionization-tandem mass spectrometry (ESI-MS/MS). An aliquot of the purified protein was separated by SDS-PAGE and digested enzymatically in-gel using porcine trypsin following standard protocols. Tryptic peptides were reconstituted in 5% acetonitrile/0.1% formic acid/94.9% water, separated on a C-18 column and analyzed using an Applied Biosystems API-QSTAR™ LC/MS/MS system. Full scan Q1 data was acquired by scanning from 450 to 2200 m/z, charged ions were selected for MS/MS analysis, and instrument software was used to determine the amino acid sequence of selected ions. The following sequence was determined from a tryptic fragment of Nicotianalisin: FGYATGTAIGIAPK (SEQ ID NO: 31). When the resultant sequence was used to search for homologues in an NCBI BLASTp search (Altschul et al., 1997), the top 18 matches producing significant alignments with 61-81% identity, were all to plant subtilisin-like proteases.

Molecular weight determination: The average molecular mass of the purified protease was determined using matrix-assisted laser/desorption ionization time-of-flight (MALDI-TOF) mass spectrometry using an Applied Biosystem's DE-PRO Biospectrometry™ Workstation. An aliquot of the protease was mixed with an equal volume of 10 mg/ml sinapinic acid (Sigma) matrix solution in 0.1% TFA:acetonitrile (2:1 v/v). The MALDI-TOF MS spectra were acquired at an accelerating voltage of 25 kV and in the positive ion mode.

The average molecular mass of the purified, mature protease was 78955 Da as determined by MALDI-TOF mass spectrometry. This mass concurs well with other plant subtilisin-like proteases that have been reported (Jorda et al., 1999; Meichtry, Amrhein, and Schaller, 1999; Tornero, Conejero, and Vera, 1996; Yamagata et al., 1994).

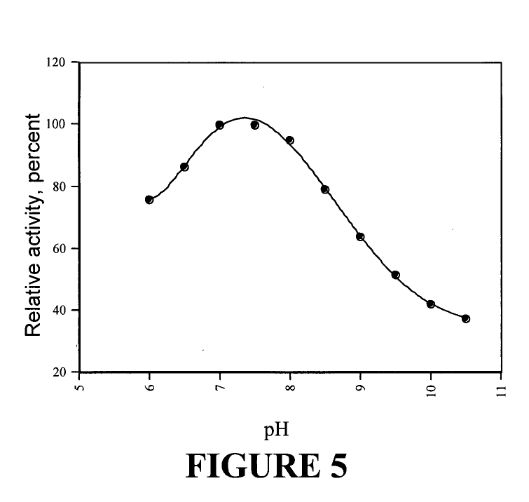

Effect of pH on the activity of purified Nicotianalisin: The purified N. benthamiana protease exhibited activity in a broad pH range with optimal enzyme activity at pH 7-7.5 (FIG. 5). Nicotianalisin was very stable in the pH range of 5.0 to 9.0. The assays were conducted in 0.1 M bis-Tris propane containing 5 mM CaCl₂ at pHs ranging from 6 to 10.5 for 30 min at 37° C. N-Suc-AAPF-pNA SEQ ID NO: (Del Mar et al., 1980; Del Mar et al., 1979) was used as a substrate at 0.3 mM in the reaction mixture as described above. The absorbance of the p-nitroaniline produced was measured at 410 nm. Values are shown as percentages of the maximum activity.

Nicotianalisin Protease Substrate Specificity:

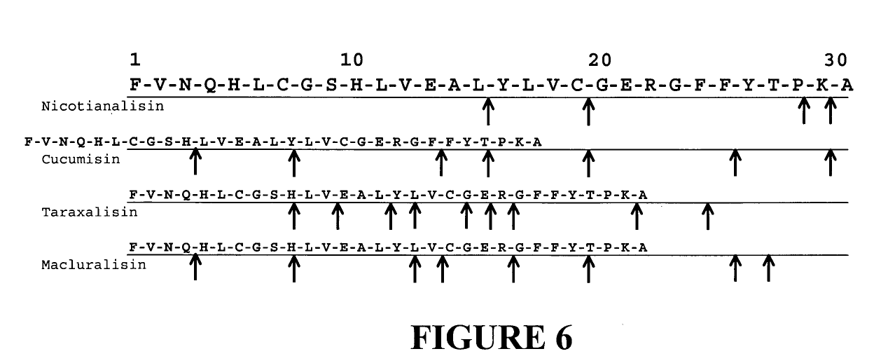

Protease-mediated hydrolysis of bovine insulin B chain. An aliquot of the pure protease was mixed with oxidized insulin B chain protein (Sigma) (5 mg/ml) and incubated for 2 to 16 hr at 37° C. The reactions were stopped by addition of 0.1% TFA in 50% acetonitrile and the mass of the cleavage products was determined by MALDI-TOF mass spectrometry as described above. As indicated in FIG. 6, hydrolysis occurred after leucine, cysteine, proline, and lysine amino acid residues. Nicotianalisin protease also showed a preference for large hydrophobic residues in the P₃ and P₄ positions of insulin B-chain with respect to Leu-15, Cys-19, Pro-28, and Lys-29 (FIG. 6). This is a common biochemical characteristic of other plant subtilisin-like proteases (Rudenskaya et al., 1998). Nicotianalisin enzyme specificity. The enzyme specificity of Nicotianalisin was assessed by monitoring the cleavage efficiency of numerous chromogenic synthetic substrates. An aliquot of the purified protease was added to an assay mixture containing 0.1 M Tris-HCl, pH 7.0, 5 mM CaCl₂, 0.5-2.7% dimethylformamide, and 0.3 to 2 mM synthetic substrate and allowed to proceed for 30 min at 37° C. The released p-NA moiety was measured at 410 mu as previously described. Relative activity was reported as a percentage activity of Nicotianalisin in the presence of a given substrate compared to N-Suc-AAPL-pNA SEQ ID NO: substrate at a given substrate concentration.

As summarized in Table 3, purified Nicotianalisin preferentially cleaved the synthetic peptide substrates N-Suc-Ala-Ala-Pro-Leu-p-NA SEQ ID NO:, N-Suc-Ala-Ala-Pro-Met-p-NA SEQ ID NO:, and N-Suc-Ala-Ala-Pro-Phe-p-NA SEQ ID NO:, after the Leu, Met, and Phe amino acid residues, respectively. This is in close agreement with the activities of other plant subtilisin-like proteases that have been reported (Kaneda, Yonezawa, and Uchikoba, 1995; Rudenskaya et al., 1998; Uchikoba, Yonezawa, and Kaneda, 1995). In general, Nicotianalisin had a strong primary preference for hydrophobic amino acids at scissile bonds and extended peptide substrates. Presence of hydrophobic residues Val and Tyr at P₃ and P₄ positions increased the activity at cleavage sites several fold (Substituent-NH—P₄—P₃—P₂—P₁—P₁′—COO-substituent SEQ ID NO:).

Table 3. Hydrolysis of homologous synthetic 4-nitroanilide peptides by Nicotianalisin. Enzymatic assays were conducted at 0.3 mM substrate concentration in 0.1 M Tris-HCl, pH 7.0, containing 5 mM CaCl₂, and 0.5-2.7% dimethylformamide at 25° C. Substrate specificity is reported as a percentage of activity of Nicotianalisin in the presence of a given substrate relative to the activity in the presence of N-Suc-AAPL-pNA SEQ ID NO: substrate.

| Substrate | Relative Activity | |
|---|---|---|
| N-Suc-AAPL-pNA | 100 | SEQ ID NO: |
| N-Suc-AAPM-pNA | 100 | SEQ ID NO: |
| N-Suc-AAPF-pNA | 67 | SEQ ID NO: |
| N-Suc-AAPA-pNA | 24 | SEQ ID NO: |
| N-Suc-AAPV-pNA | 8 | SEQ ID NO: |
| N-Suc-AAPD-pNA | 16 | SEQ ID NO: |
| N-Suc-AAVA-pNA | 20 | SEQ ID NO: |
| N-Suc-AAPI-pNA | 8 | SEQ ID NO: |
| N-Suc-YVAD-pNA | 70 | SEQ ID NO: |
| N-Suc-IEGR-pNA | 18 | SEQ ID NO: |
| N-Suc-AAA-pNA | 2 | |
| N-Suc-AAV-pNA | 5 | |
| N-Suc-GGG-pNA | 1 | |
| N-Suc-GGF-pNA | 3 | |
| N-Suc-GGL-pNA | 1 | |
| N-Suc-GFG-pNA | 1 | |
| N-Suc-GPK-pNA | 2 | |
| N-Suc-VGR-pNA | 3 | |
| N-Suc-YLV-pNA | 0 | |
| N-Suc-FVR-pNA | 1 | |
| N-Suc-PFR-pNA | 1 | |
| N-Suc-F-pNA | 1 | |
| N-Suc-M-pNA | 8 | |
| N-BZ-C-pNA | 2 | |
| N-BZ-Y-pNA | 20 | |
| N-BZ-R-pNA | 1 | |

Nicotianalisin Protease Substrate Specificity:

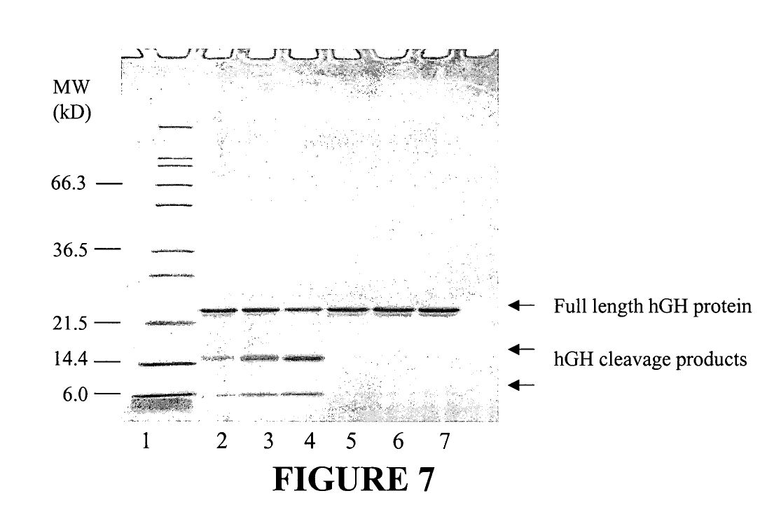

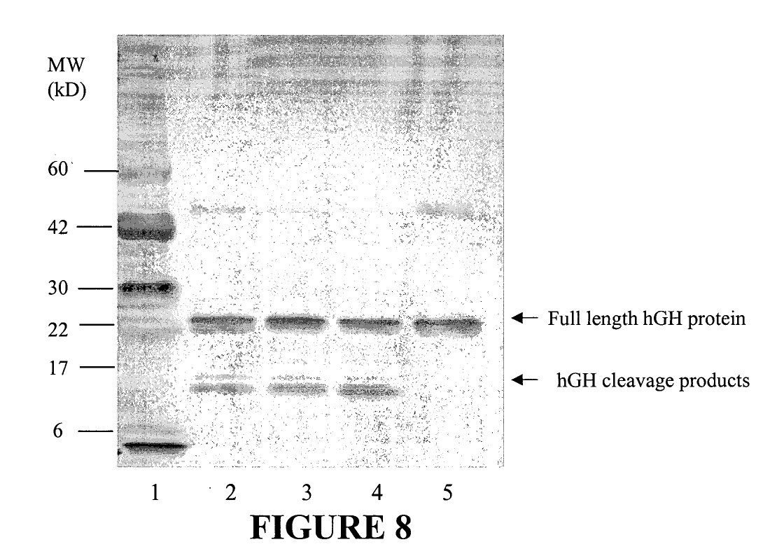

A comparison of the proteolytic activity of purified Nicotianalisin and in vivo proteolysis on hGH. To complement the results of the data in FIG. 1, purified Nicotianalisin protein was incubated with purified hGH protein in an in vitro assay, and analyzed by SDS-PAGE and Western blotting using hGH antibody. FIG. 7 shows a Coomassie stained gel of purified hGH cleaved by purified protease at 30° C. for 10, 20, or 30 min in the presence or absence of protease. Lane 1 contains protein molecular weight markers. Lanes 2, 3, and 4 represent hGH cleavage after 10, 20, and 30 min by Nicotianalisin respectively. The faint higher molecular weight bands may constitute Nicotianalisin. Controls for 10, 20 and 30 min incubations are shown in lanes 5, 6, and 7 respectively. Similarly, in FIG. 8 lane 1 contains pre-stained protein molecular weight markers. Lanes 2, 3, and 4 represent hGH cleavage by Nicotianalisin after 10, 20, and 30 min respectively. hGH after 30 min incubation at 37° C. in the absence of protease is shown in lane 5 as a control. Two prominent immunoreactive degradation products were detected (FIGS. 7 and 8) that are very similar in size to those observed after in vivo proteolysis in plants expressing hGH from a viral vector as shown in FIG. 1. The degradation products are also very similar in size to those reported in mammalian systems. A reduction in the amount of possible hGH dimmer is also observable in lane 5 compared to lanes 2, 3 and 4. Data from the in vitro cleavage studies and the characterization of the proteolytic activity of the Nicotianalisin protease clearly demonstrate that the protease identified from the IF is involved in the degradation of the hGH protein in vivo.

### EXAMPLE 2

Cloning a Family of N. benthamiana Subtilisin-Like Protease cDNA Fragments.

RT-PCR amplification of Plant Subtilisin-Like Protease Catalytic Domains from N. benthamiana and Arabidopsis.

Several factors were considered when the PCR primers were designed to clone a fragment of the N. benthamiana subtilase gene. Biochemical characterization of the purified Nicotianalisin protein revealed high identity with a plant subtilisin-like serine protease. N-terminal sequence data supported that observation and indicated homology between Nicotianalisin and the tomato subtilase family. Plant subtilisin-like genes include a 15-member tomato gene family (Meichtry, Amrhein, and Schaller, 1999) and others (Batchelor et al., 2000; Jorda et al., 1999; Jorda, Conejero, and Vera, 2000; Neuteboom et al., 1999; Yamagata et al.,(2000) Berger, 2000 #6112). Based on the fact that all members of the subtilisin family contain a highly conserved catalytic domain (Siezen and Leunissen, 1997), primers were designed to the tomato catalytic triad and used to amplify an internal fragment of the N. benthamiana subtilisin-like serine protease gene. In addition, to test the hypothesis that this is a general method for cloning similar genes from other plants, the tomato primers were also used to amplify an internal fragment of a subtilisin-like protease from Arabidopsis thaliana.

Total RNA was prepared from whole plants by the hot borate RNA extraction method (Krieg, 1996). Nucleotide sequence from the tomato subtilisin-like protease (Tornero, Conejero, and Vera, 1996) was used to design PCR primers to amplify a conserved catalytic domain. The non-degenerate tomato primer 5′ GTG AGG GCA AGA CAT TGA TGT GCC TGA TAT GAT ATT GAA 3′ SEQ ID NO: 1, was used for first strand synthesis using the RETROscript™ RT-PCR kit (Ambion, Austin, Tex.). PCR amplification of the cDNA proceeded with the addition of the primer, 5′ GGC GTG ATT ATC GGA GTT ATA GAC 3′ SEQ ID NO:2, in a Perkin-Elmer 2400 GeneAmp PCR System for 35 cycles, each consisting of 94° C., for 20 sec, 40° C. for 30 sec and 72° C. for 40 sec.

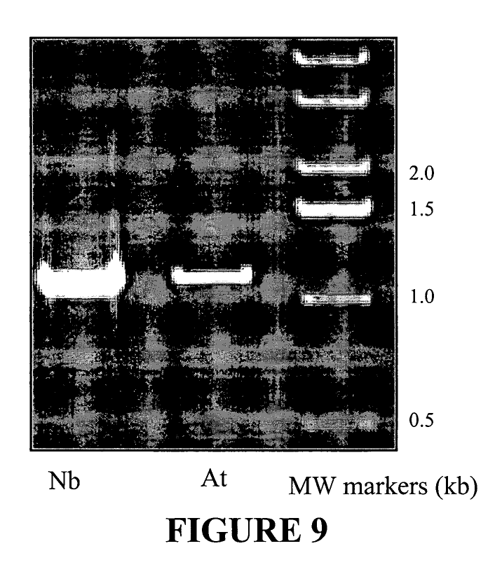

The RT-PCR amplified N. benthamiana and A. thaliana DNA fragments were visualized in agarose gels by ethidium bromide staining and a single DNA band of approximately 1200 bases was observed for both PCR products (FIG. 9). The size of these fragments concur with the tomato (Tornero, Conejero, and Vera, 1996) and the Arabidopsis (Ribeiro et al., 1995) reported sequences for this region.

cDNA Cloning and Sequencing. Following amplification of the appropriately-sized bands (FIG. 9), fragments from both N. benthamiana and A. thaliana were cut from the gel and DNA cloned into the pCR® 2.1-TOPO® vector as per manufacturers instructions (Invitrogen, Carlsbad, Calif.). Sequencing of all cDNA clones was performed using the ABI 310 Genetic Analyzer (Applied Biosystems, Foster City, Calif.). The sequence was further analyzed using the Sequencher software program. The nucleotide and deduced amino acid sequences were analyzed using DNAMAN Sequence Analysis Software (Lynnon BioSoft).

The sequences of the N. benthamina and A. thaliana PCR fragments were homologous to the tomato (Tomero, Conejero, and Vera, 1996) and Arabidopsis (Ribeiro et al., 1995) subtilisin-like proteases. Sequence alignment of the N. benthamina gene fragments, NbP2-NbP7, revealed that they represent two contigs. NbP6 alone is one contig and the other five clones (NbP2, NbP3, NbP4, NbP5, and NbP7) form the second contig. Therefore, the NbP3 (SEQ ID NO: 3) and NbP6 (SEQ ID NO: 4) sequences were used to represent the two different genes.

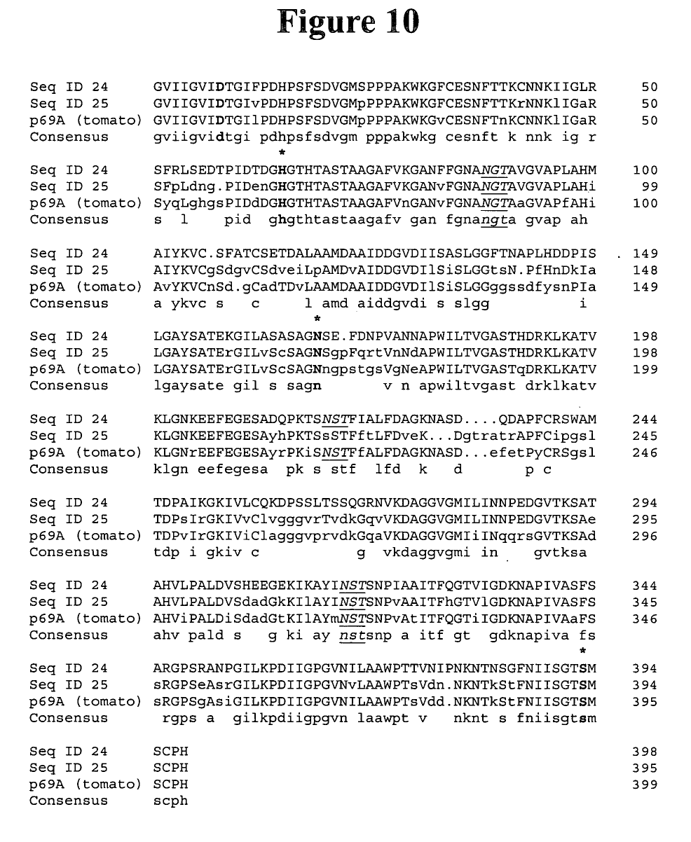

The deduced amino acid sequences of SEQ ID NO: 3 and SEQ ID NO: 4 (i.e., SEQ ID NO: 24 and SEQ ID NO: 25, respectively) have characteristic motifs that are shared with other plant subtilisins. Three potential asparagine-linked glycosylation sites that are present in p69A and conserved in the tomato family (Meichtry, Amrhein, and Schaller, 1999), are also conserved in SEQ ID NO: 24 (FIG. 10). SEQ ID NO: 25 has two of the conserved N-glycosylation sites (FIG. 10). The Asp-His-Ser catalytic triad that is conserved in other subtilisins, including the tomato family (Meichtry, Amrhein, and Schaller, 1999), is also conserved in N. benthamiana (FIG. 10). In addition, SEQ ID NO: 24 and 25 both contain the highly conserved and catalytically important Asn residue that is known to stabilize the tetrahedral transition state of the enzyme reaction of subtilisin-like proteases (Gensberg, Jan, and Matthews, 1998; Siezen and Leunissen, 1997).

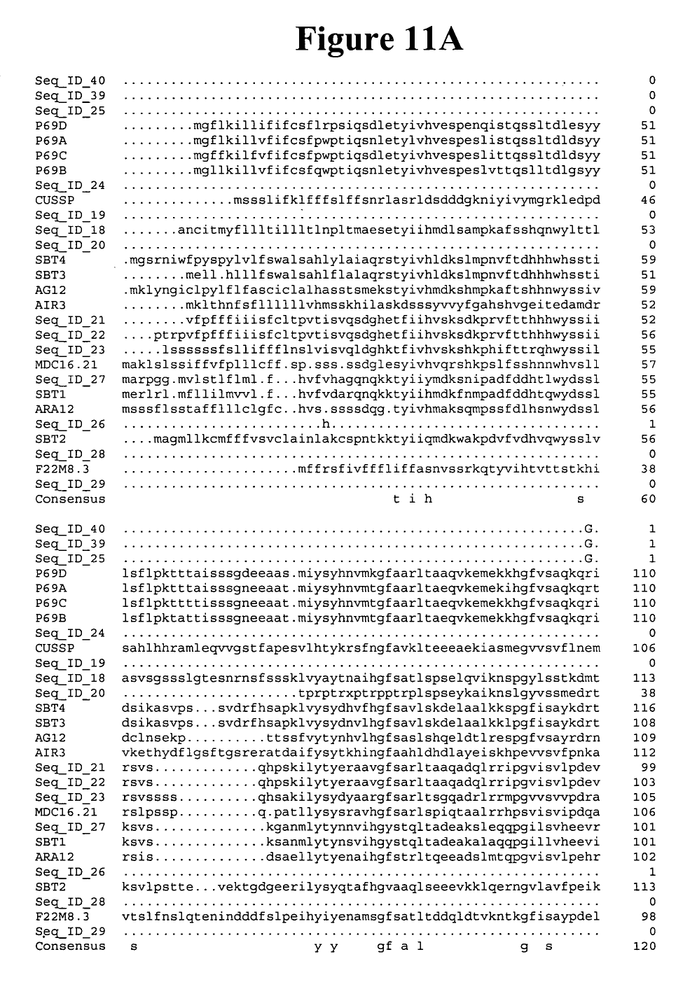

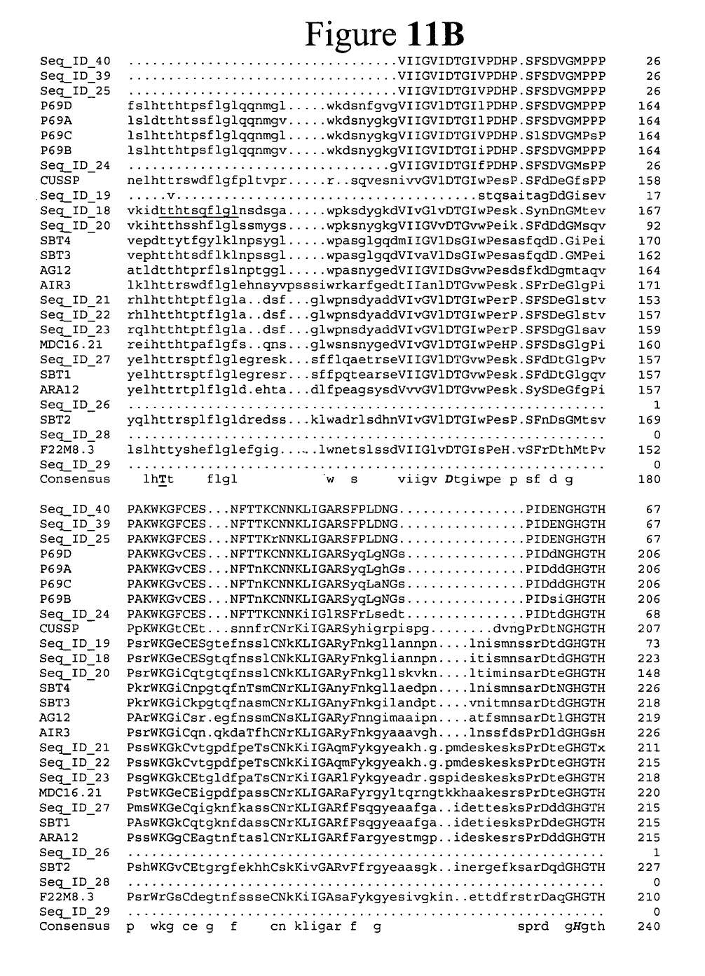

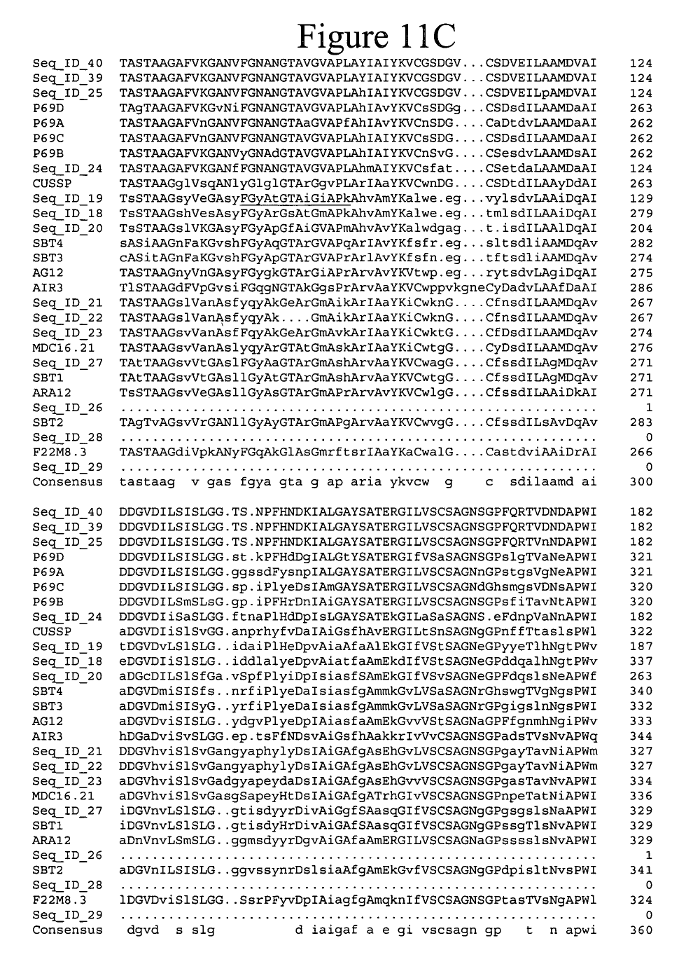

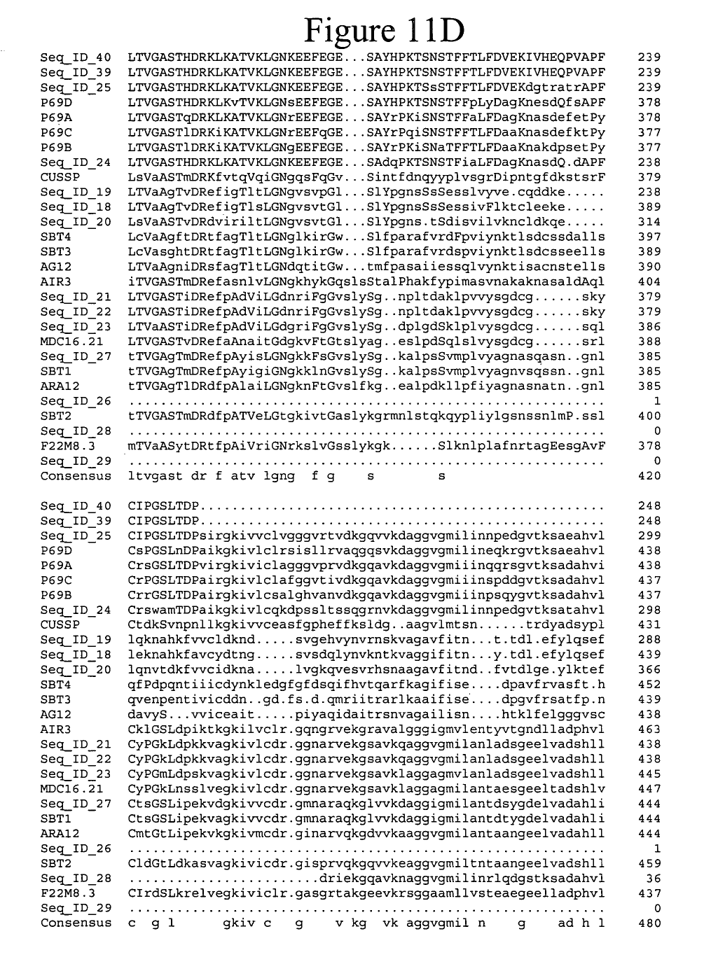

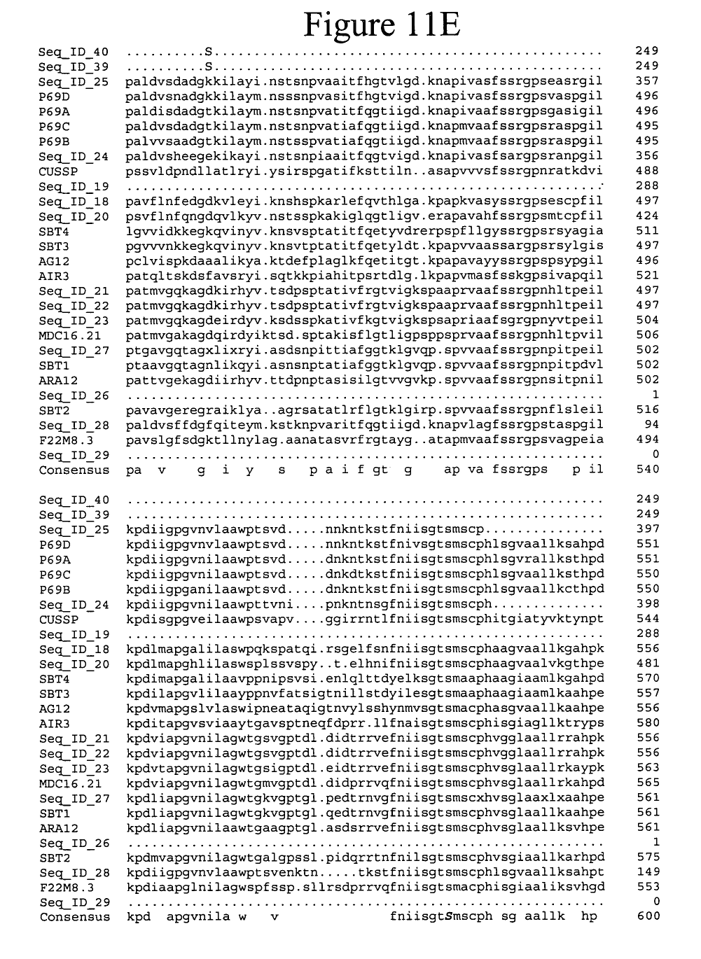

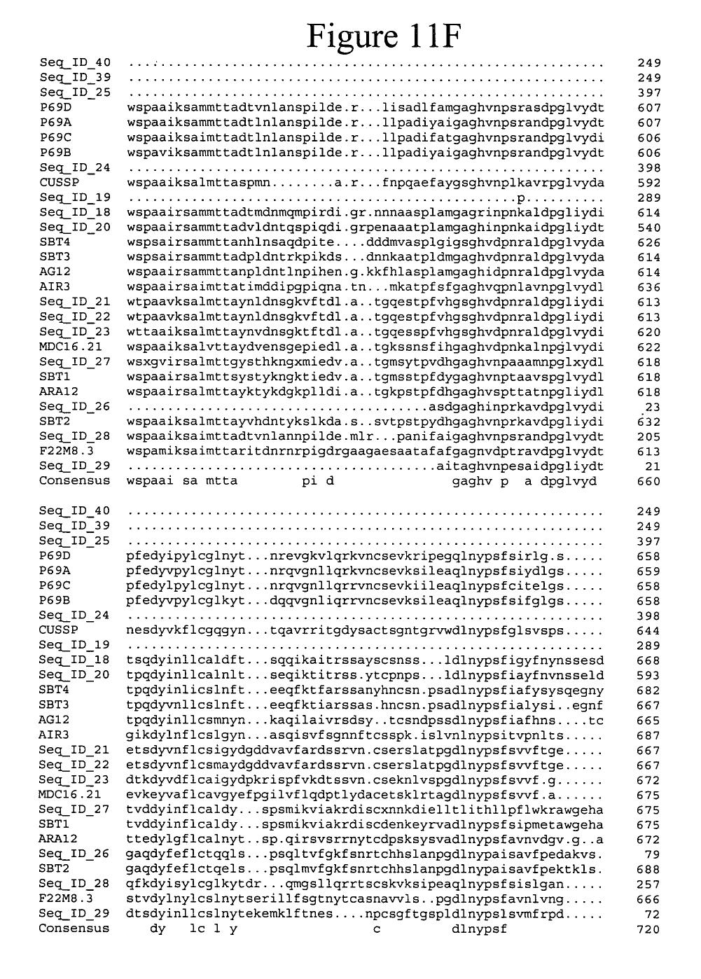

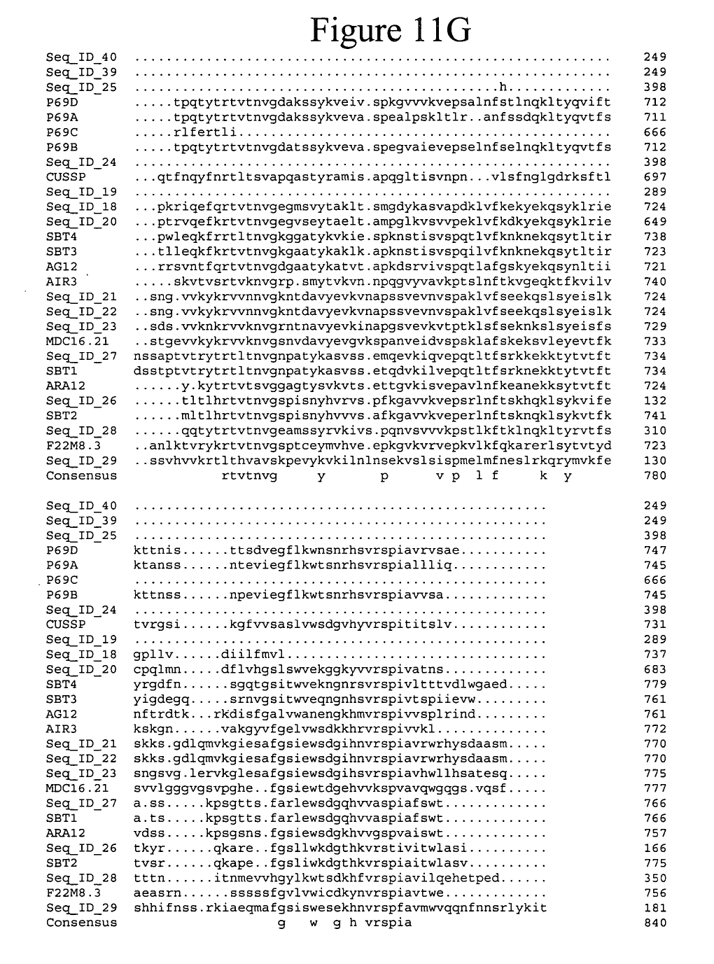

The tomato non-degenerate primers used in this study amplified both the N. benthamiana and the A. thaliana subtilisin-like gene fragments. Sequence analysis revealed 83% deduced amino acid residue identity between the tomato p69A subtilase gene fragment (Tomero, Conejero, and Vera, 1996) and the N. benthamiana subtilisin-like serine protease gene fragments. In the case of the Arabidopsis gene fragment, SEQ ID NO: 1 primer sequence was approximately 90% identical to the reported Arabidopsis sequence (Ribeiro et al., 1995). The deduced amino acid sequence covering this primer region is also highly conserved in all plant subtilisin-like proteases (FIG. 11). The conservation of the sequence may allow this method to be used to clone subtilisin-like proteases that have not yet been identified from other organisms.

### EXAMPLE 3

Down-Regulation of Endogenous N. benthamiana Subtilisin-like Serine Protease Activity Using a Plant Viral.

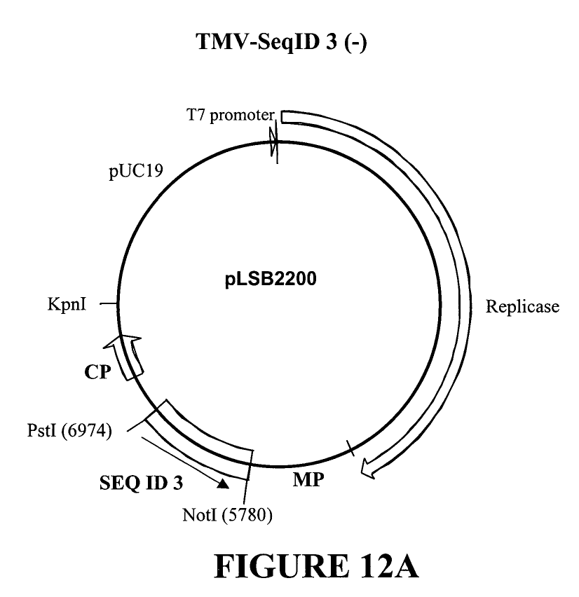

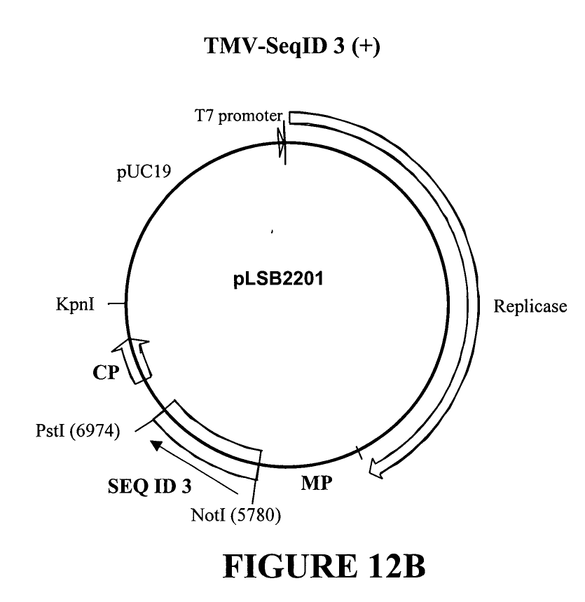

Antisense and sense expression of a N. benthamiana subtilisin-like gene fragment in a TMV-based viral vector. Viral vectors have been shown to induce gene silencing in plants (Baulcombe, 1999; Lindbo, Fitzmaurice, and della-Cioppa, 2001). In addition, plant metabolic pathways have been altered via the delivery of viral-vector mediated gene silencing (Kumagai et al., 1995). To inhibit the endogenous proteolytic activity of Nicotianalisin in vivo, SEQ ID NO: 3, a partial cDNA sequence of the N. benthamiana subtilisin-like protease, was placed under control of the TMV-U1 coat protein subgenomic promoter in both the sense and antisense orientation (FIGS. 12A and B). Unique Pst I/Not I and No I/Pst I restriction sites were added at the 5′ and 3′ ends, respectively, of the SEQ ID NO: 3 gene fragment using PCR mutagenesis. The fragments were subcloned into TMV expression vectors in the antisense and sense orientation using methods that have been previously described (Kumagai et al., 1995), generating the TMV silencing vectors, pLSB2200 and pLSB2201 (FIGS. 12A and 12B). In addition, green fluorescent protein (GFP) was cloned into a TMV expression vector in the sense orientation and used as a control vector. Infectious RNA was generated using an mMESSAGE mMACHINE™ capped RNA transcription kit (Ambion, Austin, Tex.) as per manufacturer's recommendations and used to inoculate N. benthamiana plants as previously described (Kumagai, et al., 1995; McCormick et al., 1999). At 10 days post-inoculation leaves were harvested, and the plant IF fraction was isolated. Aliquots of the plant IF extracts were assayed for inhibition of protease activity using the synthetic substrate, N-Suc-Ala-Ala-Pro-Leu-p-NA SEQ ID NO:, and substrate-imbedded Zymogram gels as described in Example 1. The protease activities of IF extracted from GFP-inoculated plants and non-inoculated plants were used as controls.

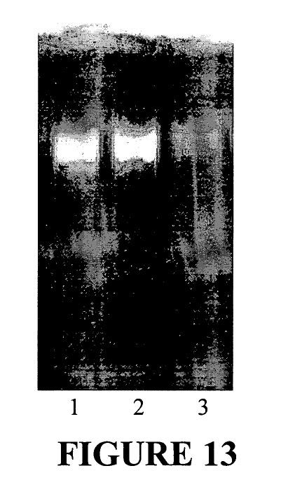

Plant viral vector, pLSB2200 (FIG. 12A), expressing the N. benthamiana subtilisin-like protease gene fragment in the antisense orientation, mediated the down regulation of Nicotianalisin protease activity in N. benthamiana leaves. FIG. 13, shows a significant reduction of protease activity in the IF from an infected plant (lane 3) compared to the activity in IF from an uninoculated plant (lane 1) or a control plant infected with a viral vector expressing GFP (lane 2) as assessed by the in situ protease gel assay (see assay description in Example 1).

The inhibition of protease activity was also observed in plants inoculated with the sense construct, pLSB2201 (FIG. 12B). As summarized in Table 4, a similar reduction of protease activity was observed in plants inoculated with either sense or antisense infectious RNA as compared to uninoculated or GFP vector control plants.

Table 4. Percent reduction protease activity. IF was prepared from virally-infected and uninoculated plant leaves. Proteolytic activity was monitored by the hydrolysis of the synthetic substrate, N-Suc-AAPL-p-NA SEQ ID NO: as described in Example 1. Reduction of the protease activity was reported as the relative activity as compared to the activity in the IF from an uninfected plant (control).

| Plant Vector | Relative Activity |
|---|---|
| Uninoculated | 100% |
| GFP vector control | 100% |
| pLSB2201 (sense) | 40% |
| pLSB2200 (antisense) | 30% |

This result is similar to a previous study that demonstrated a manipulation of the carotenoid biosynthetic pathway in plants using plant viral vectors expressing gene and gene fragments in both the sense and antisense orientation (Kumagai et al., 1995).

### EXAMPLE 4

Down-Regulation of N. benthamiana Subtilisin-Like Serine Protease Activity Using TRV and TMV Plant Viral Vectors.

Tobacco rattle virus (TRV) has also been shown to mediate gene silencing in plants (Ratcliff, Martin-Hernandez, and Baulcombe, 2001; Ratcliff, MacFarlane, and Baulcombe, 1999). The upper leaves emerging after infection show little to no viral symptoms, but still exhibit post-transcriptional gene silencing of nuclear genes (Ratcliff, MacFarlane, and Baulcombe, 1999). In addition, it has been shown that these upper leaves can be re-inoculated with another, distinctively different plant viral vector that can express a heterologous protein. Therefore, a strategy was developed utilizing both TRV and TMV to silence endogenous Nicotianalisin protease activity and express recombinant human growth hormone protein in planta.

Construction of TR V-SEQ ID NO: 3 sense and antisense. Tobacco rattle virus (TRV) RNA-2 encodes a capsid protein and two non-structural proteins, 2b and 2c. RNA-2 is not essential for infection in plants. It has been previously modified for expression of heterologous proteins. In this example, construct TRV-GFP (MacFarlane and Popovich, 2000), which has the 2b and 2c genes of TRV RNA-2 replaced with the pea early browning virus (PEBV) coat protein subgenomic promoter, was modified by PCR-directed mutagenesis. Oligonucleotides (5′-GTCCTAATCCCTAGGGATTTAAGG-3′ SEQ ID NO: 32, upstream, TRV2AVR2) and (5′-CTTTGGAAATTGCAGAAAC-3′ SEQ ID NO: 33, downstream, TRV4307-4289) were used to PCR amplify the region between the Avr II and Pst I sites of plasmid TRV-2b-GFP (MacFarlane and Popovich, 2000), which is similar to TRV-GFP except that it retains the 2b gene. Oligonucleotides (5′-GTTTCTGCAATTTCCAAAG-3′ SEQ ID NO: 34, upstream, TRV4289-4307) and (5′-GAATTCGGGGTACCGCGGCCGCGATATCCTGCAGGGCGTTAACTC-3′ SEQ ID NO: 35, downstream, TRVPST/NOT PL) were used to PCR amplify the region between the Pst I site and the 3′-end of the PEBV coat protein subgenomic promoter of construct TRV-2b-GFP. The two resulting PCR fragments were then joined by splice overlap PCR using oligonucleotides TRV2AVR2 and TRVPST/NOT PL and cloned into TRV-GFP digested with Avr II and Kpn I. The resulting construct, pK20-2b-P/N-SmaI, includes the 2b gene and has unique Pst I, EcoRV, and Not I cloning sites, with a Sma I site at the 3′-terminus of the TRV RNA-2 cDNA insert. Construct pK20-2b-N/P-SmaI, in which the Pst I and Not I sites were reversed, was constructed as described above, except oligonucleotide (TRVNOT/PST PL, 5′-GAATTCGGTACCCTGCAGGATATCGCGGCCGCGGCGTTAACTCGG-3′ SEQ ID NO: 36) was used instead of oligonucleotide (TRVPST/NOT PL SEQ ID NO: 35).

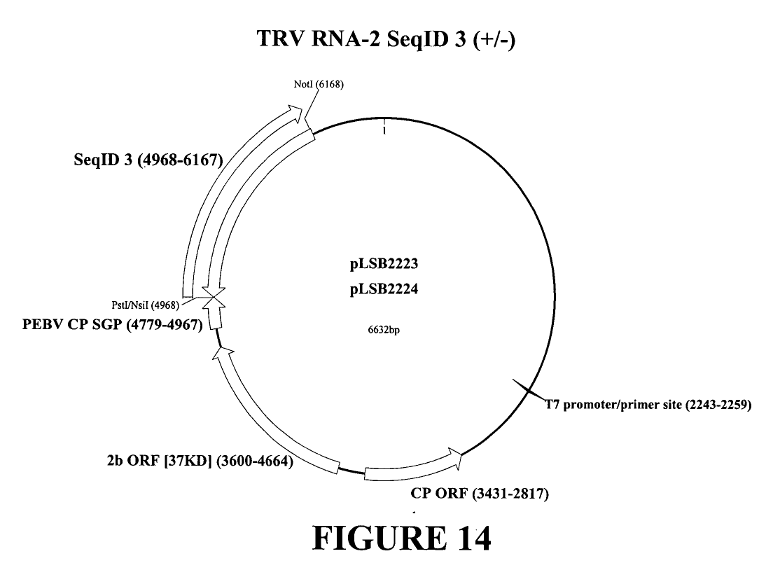

The subtilisin-like protease cDNA from N. benthamiana containing unique Nsi I and Not I sites at the 5′ and 3′ ends, respectively, was PCR amplified from plasmids pLSB2201 (sense orientation) and pLSB2200 (antisense orientation) using the following oligonucleotides (5′-TGGTTCTGCAGTTATGCATAGGCGTGATTATCGGAGTTATAG-3′ SEQ ID NO: 37, upstream) and (5′-TTTCCTTTTGCGGCCGCGTGAGGGCAAGACATTGATG-3′ SEQ ID NO: 38, downstream). The subtilisin protease gene fragment was then subcloned into the Pst I/Not I sites of pK20-2b-P/N-SmaI in the sense orientation and the Not I/Pst I sites of pK20-2b-N/P-SmaI in the antisense orientation. The resultant TRV RNA2-SEQ ID NO: 3 sense and antisense constructs were pLSB2223 and pLSB2224, respectively (FIG. 14).

Accumulation of hGH in a dual-virus expression system. TRV RNA2-SEQ ID NO: 3 sense construct pLSB2223 (FIG. 14), was linearized with Sma I and transcribed using T7 RNA polymerase (Ambion mMessage mMachine). N. benthamiana plants were inoculated with a mixture of transcript of RNA2 with transcripts from a full-length clone of TRV RNA-1. Transgenic plants expressing GFP were also used as a control to monitor gene silencing using a vector carrying gfp in RNA-2.

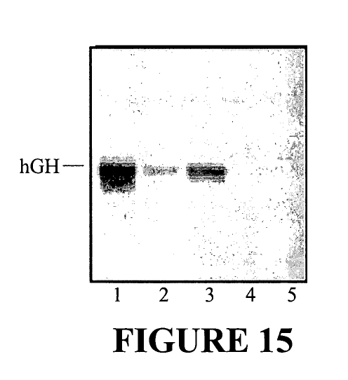

At 9 days post-inoculation the GFP expression in the GFP transgenic plants that were inoculated with TRV-2b-GFP RNA2 was silenced. Therefore, TRV RNA2-SEQ ID NO: 3-infected plants were then inoculated with a TMV-expression vector containing the hGH gene as described in Example 1. Eight to 10 days post TMV inoculation, plants were analyzed for hGH accumulation by Western immunoblot. The effect of in vivo reduction of Nicotianalisin activity by the recombinant TRV on the accumulation of a recombinant protein (hGH) expressed by a TMV vector is represented in FIG. 15. In the pLSB2223 TRV RNA2 SEQ ID NO: 3 infected plants, intact hGH accumulation (Lane 3, FIG. 15), was significantly higher than in plants not previously infected with TRV RNA2 SEQ ID NO: 3 (Lane 2, FIG. 15) suggesting that endogenous Nicotianalisin protease activity was down regulated.

### EXAMPLE 5

N. benthamiana Subtilisin Gene Family and Strategies to Suppress the Protease Activity.

Isolation and characterization of N. benthamiana cDNAs homologous to the subtilisin-like protease. Several N. benthamiana cDNA libraries were constructed using whole plant, roots, apical meristem, and flowers of N. benthamiana. Individual clones were picked randomly, partially sequenced (resulting in an expressed sequence tag; EST), annotated and the information deposited in a searchable database. Several cDNAs, in addition to SEQ ID NO: 3 and SEQ ID NO: 4, with homology to other subtilisin-like proteases were found following querying of the database. These thirteen additional cDNAs are shown in Table 6 with their corresponding clone names: 28965 (SEQ ID NO: 5). 48994 (SEQ ID NO: 6), 103775 (SEQ ID NO: 7), 103965 (SEQ ID NO: 8), 108459 (SEQ ID NO: 9), 111767 (SEQ ID NO: 10), 113167 (SEQ ID NO: 11), 114340 (SEQ ID NO: 12), 155186 (SEQ ID NO: 13), 266847 (SEQ ID NO: 14), 272011 (SEQ ID NO: 15), 272344 (SEQ ID NO: 16), and 274641 (SEQ ID NO: 17). With the exception of SEQ ID NO: 12, both partial and full open reading frames (ORF) of SEQ ID NO: 3 to 17 were generated based on the homology to the Genbank sequences. SEQ ID NO: 12 produced several disrupted ORFs that may indicate that it is a pseudogene. However, additional data are needed in order to determine the role of this ORF. These fourteen Nicotianlisins (SEQ ID NO: 18-29, and 39-40) were aligned against fourteen homologous subtilisin-like proteases, and the results are shown in FIG. 11. The two peptide sequences derived from the purified protease fraction isolated from the plant IF (FIG. 11, SEQ ID NO: 30 and SEQ ID NO: 31) were found to match the deduced amino acid sequences of two different cDNAs: SEQ ID NO: 7 and SEQ ID NO: 17. As the deduced amino acid sequence of SEQ ID NO: 7 (i.e., SEQ ID NO: 19) lacks the N-terminal portion that would be examined for identity with the peptide sequence SEQ ID NO: 30, the possibility cannot be excluded that both SEQ ID NO: 30 and SEQ ID NO: 31 might be found in a deduced amino acid sequence of a full-length version of the gene represented by SEQ ID NO: 7. However, the deduced amino acid sequence of SEQ ID NO: 17 (i.e., SEQ ID NO: 18) contains the genetic regions correlating with both peptides, but was only identical with SEQ ID NO: 30, and not with SEQ ID NO: 31. This indicates that the IF of N. benthamiana leaves accumulates proteases expressed from at least one, and more likely two genes.

The gene represented by SEQ ID NO: 17 contained a partial open reading frame (ORF) of 2196 base pairs, coding for a polypeptide of minimum 737 amino acids and a deduced processed polypeptide of 620 amino acid with predicted molecular mass of 66,915 Da.

All fifteen sequences were aligned using the DNAMAN sequence analysis program, and the alignment and the deduced phylogenetic tree, are shown in FIGS. 10A and 10B. Based on this alignment, different variable and conserved regions of the sequences (FIG. 16A) were chosen as targets for making RNAi constructs to reduce the protease expression in plants (Table 6). There are two blocks of conserved sequence (Regions A and B) found in the alignment (FIG. 16A). Genes that share≧74% homology were grouped into single clusters. This resulted in eight different clusters representing 15 genes (Table 6). The variable regions represent a sequence that is unique to an individual gene. The conserved regions represent similar regions among closely related genes and, therefore, this sequence may be used to target genes in the same cluster for silencing.

In addition, the MALDI-TOF mass spectrum (see Example 1) concludes that the protein appears to be glycosylated. The mass data indicates that Nicotianalisins are 15% glycosylated. Percent glycosylation was calculated based on the mass difference between the theoretical and measured mass of isolated Nicotianalisin as determined by MALDI-TOF. The size of the mature protease and the presence of theoretical glycosylation sites in the deduced amino acid sequence concur with other plant subtilisin-like proteases that have been reported.

### EXAMPLE 6

Reduction of Plants' Subtilisin-Like Protease Activities by Expression of Sense and Antisense of Subtilisin Genes.

Viral induced gene silencing approaches. Foreign proteins expressed in N. benthamiana via viral vectors are sometimes degraded by plant proteases. We found that the IF from the leaf possesses at least two subtilisin-like peptides that are associated with protease activities. These peptide sequences were present in clones SEQ ID NO: 7 and SEQ ID NO: 17. In order to facilitate the accumulation of foreign proteins in plants, plants are inoculated with a TRV vector containing a piece of a subtilisin-like gene. Since TRV is a strong silencing vector, this initiates the silencing of endogenous subtilisin gene(s). Then, these plants are inoculated further with a TMV vector containing the desired protein gene. One can attempt to selectively silence each individual gene using a unique sequence in the variable region, or one can concatenate two or more units to silence more genes. In addition, one can silence related genes using the conserved unit for each cluster. Again, one can concatenate these conserved units to silence several groups or all of the subtilisin genes. One can selectively use a combination of variable and/or conserved units to obtain a desirable trait (accumulation of protein) while limiting possible undesirable effects of reduced expression of one or more protease genes on the plant's growth.

Transgenic plant approaches. The gene silencing strategy can also be used in stably transformed plants (e.g., via Agrobacterium-mediated transformation, transposons and other genome integrating vectors) expressing these variable and conserved units. However, this type of silencing in plants is generally less effective than the transient (viral vector-mediated) method described above. Recently, expression of double-stranded RNA (ds-RNA) was found to significantly increase the efficiency and degree of silencing in stably transformed plants (Chuang and Meyerowitz, 2000; Waterhouse, Graham, and Wang, 1998). This construction typically has the sense and antisense units interrupted by a loop. This loop can be a fragment of coding region or an intron, and it is expected to form a hairpin structure following transcription. One can adapt this ds-RNA method as a more efficient way of silencing the proteases in plants. In this case, transgenic plants containing the silencing unit are inoculated with a TMV vector expressing the protein of interest. Two weeks post-inoculation, the accumulated protein is purified from leaves. Similarly to the transient method above, one can combine the effects of different units by concatenating these units into a single construct. Another way to combine these units is to sexually cross transgenic plants carrying individual or multiple units, and then screen the progeny for the presence of both transgenes. The expression of the silencing unit can be driven by any largely constitutive promoter such as those derived from CaMV 35S, actin, rubisco, or ubiquitin. However, silencing of certain unit(s) may cause aberrations or a lethal phenotype in transformed plants. To overcome problems caused by constitutive silencing of the protease(s), an inducible promoter is used to facilitate induction of silencing shortly before infection with the viral vector expressing the gene of interest, thus allowing the protein of interest to accumulate, but minimizing the time for undesirable phenotypes to develop. Promoters have been isolated that have been shown to be inducible in transgenic plants by glucocorticoid (Martinez et al., 1999), salicylic acid (Lebel et al., 1998), or copper (Mett, Lochhead, and Reynolds, 1993). In this case, the transcription of the silencing unit(s) is driven by any appropriate inducible promoter(s). Transformed plants are obtained in the absence of the inducer. In the case of targeting individual genes or subgroups, it is important to design the trigger dsRNA carefully to take into account the possibility of transitive RNAi (interfering RNA) causing unintended silencing of homologous genes (Nishikura, 2001).

Either the protein of interest, such as hGH and/or the genetic element capable of reducing protease activity maybe stably incorporated into the host genome by conventional techniques. Either direct protease inhibitors such as aprotinin or inhibitors to prevent formation of the protease may be used. When the genetic element and/or the protein of interest interfere with the functioning of the plant, either or both may be under regulatory control, which can be altered. For example, by using an inducible promoter, one can culture the transgenic plant without expression of the genetic element and/or the protein of interest. At a selected time, an inducer may be added or the conditions changed to allow the promoter to express the genetic element and/or the protein of interest. Likewise, the opposite may be done for repressors and indirect regulatory elements.

When using transgenic plants and only one of the gene for the protein of interest or the genetic element is present, the other may be added by using a vector.

TABLE 6
N. benthamiana cDNA Clones Homologous to Subtilisin-Like Proteases

| | | Conceptual | | Relative a.a. | | | |
|---|---|---|---|---|---|---|---|
| N.A. | | translated | | coverage of | Name of | Name of | Name of |
| SEQ | Clone | protein, | Homologous Protein and its | homologous | Variable | Conserved | Conserved |
| ID | Name | SEQ ID | Annotation | Protein | region | region A | region B |
| 17 | 274641 | 18 | Alnus glutinosa, S52769 | ˜8-760 | V1 | C1A | C1B |
| | | | subtilisin-like proteinase ag12 | (761) | | | |
| 7 | 103775 | 19 | Alnus glutinosa, S52769 | 150-430 | V2 | C2A | C2B |
| | | | subtilisin-like proteinase ag12 | (761) | | | |
| 11 | 113167 | 20 | Alnus glutinosa, S52769 | 60-755 | V3 | C1A | C2B |
| | | | subtilisin-like proteinase ag12 | (761) | | | |
| 8 | 103965 | 21 | A thaliana, BAB02339 | 7-770 | V4** | C4A** | C5B** |
| | | | cucumisin-like serine protease | (777) | | | |
| 5 | 28965 | 22 | A thaliana, BAB02339 | 7-770 | | | |
| | | | cucumisin-like serine protease | (777) | | | |
| 16 | 272344 | 23 | A thaliana, BAB02339 | ˜6-769 | V5 | C4A | C5B |
| | | | cucumisin-like serine protease | (777) | | | |
| 3 | NbP3 | 24 | Tomato, T06580 subtilisin-like | 140-540 | | C6A | C8B |
| | | | proteinase p69f | (747) | | | |
| 4 | NbP6 | 25 | Tomato, T06580 subtilisin-like | 140-534 | | C6A | C8B |
| | | | proteinase p69f | (747) | | | |
| 14 | 266847 | 39 | Tomato, T06580 subtilisin-like | 139-388 | V8* | C6A | C8B |
| | | | proteinase p69f | (747) | | | |
| 15 | 272011 | 40 | Tomato, T06580 subtilisin-like | 139-388 | | C6A | C8B |
| | | | proteinase p69f | (747) | | | |
| 9 | 108459 | 26 | Tomato, T07172 subtilisin-like | 614-775 | V10 | | |
| | | | proteinase | (775) | | | |
| 10 | 111767 | 27 | Tomato, T07171 subtilisin-like | 1-766 | V11 | | |
| | | | proteinase SBT1 | (766) | | | |
| 12 | 114340 | | A thaliana, AAD12260 subtilisin- | ˜150-522 | V12 | | |
| | | | like protease | (772) | | | |
| 13 | 155186 | 28 | Tomato, CAA07250 serine | 404-745 | V13 | | |
| | | | protease | (747) | | | |
| 6 | 48994 | 29 | A thaliana, AAF76468 similar to | 593-755 | V14 | | |
| | | | p69d gene of tomato | (756) | | | |

Each cluster is separated by a heavy line.
Abbreviations:
N.A.: nucleic acid.
V: Variable (unique to each sequence).
C: Conserved.
*This region would behave similar to the conserved region, and it may be used to inhibit all four genes in this group.
**This region would also be used for clone 28965 (SEQ ID NO:5) in the same group.

### EXAMPLE 7

Reduction of Plants' Subtilisin-Like Protease Degradation of a Protein by Simultaneous Expression of a Protease Inhibitor Gene.

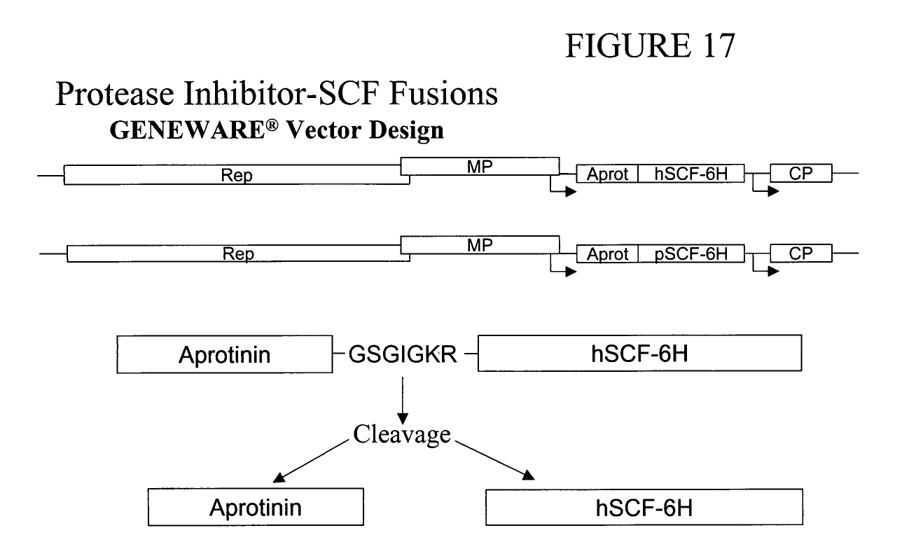

Human and porcine Stem Cell growth Factor (SCF) genes were cloned in GENEWARE® vectors either alone (neat) or fused to either a 6 histidine tag, an HDEL tag, both 6-His and HDEL or an aprotinin gene connected by a cleavable linker. The vector design for the fusion to aprotinin gene is shown in FIG. 17. Controls of a GENEWARE® vector containing gfp (clone 5), aprotinin cloned in a GENEWARE® vector, E. coli-produced recombinant hSCF purified protein and purified natural aprotinin were also prepared.

The vectors were used to inoculate plant leaves of different sets of plants as described in the examples above. Both plant homogenates and interstitial fluid were extracted as described above. hSCF and pSCF were each purified from 27 plants to yield 0.85 mg (˜15 mg/kg) and 2.5 mg (˜45 mg/kg) of protein respectively. Recovered proteins were biologically active in CD34+ proliferation assays

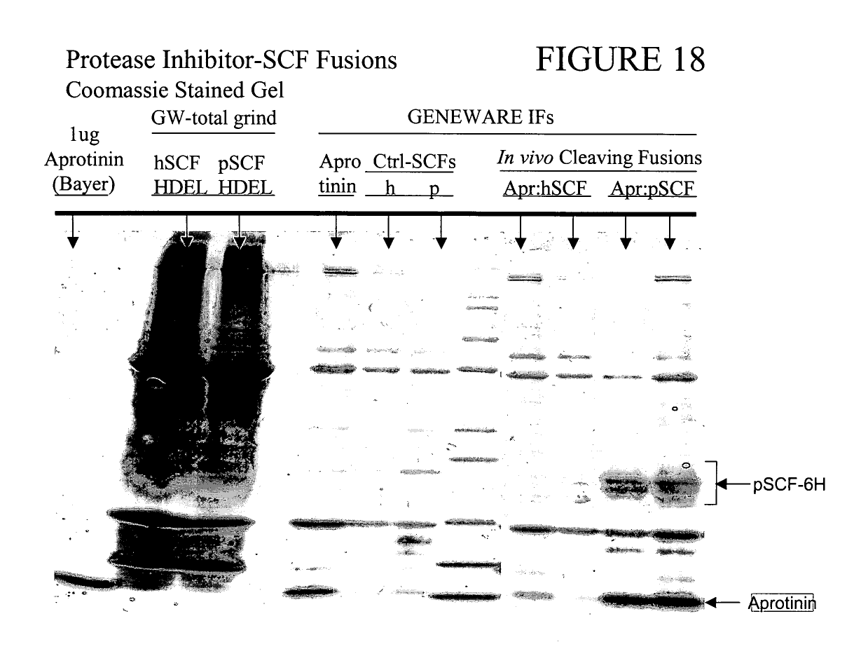

Samples from some of the experiments were taken and subjected to SDS-PAGE and stained with Coomassie Blue. The gel is shown as FIG. 18. Within the protein rich homogenate, little specific production can be seen but in the interstitial fluid (IF) fractions, it is possible to see a protein band in the location of SCF and aprotinin when using a vector expressing both genes. Such bands are not readily apparent in lanes where the vector lacked aprotinin.

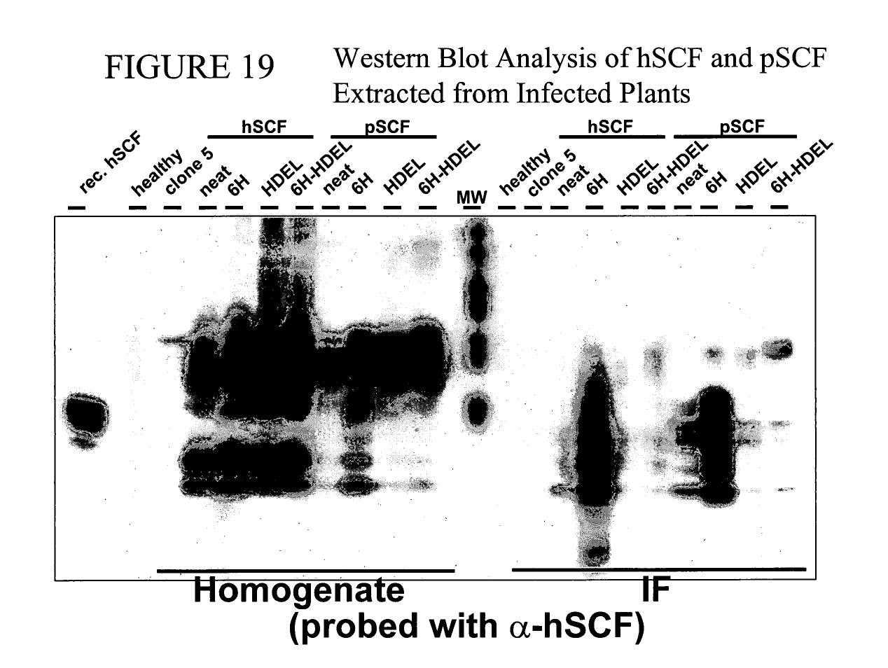

To distinguish SCF proteins from other cellular proteins of little interest, a Western blot was performed using various samples and an antibody against hSCF that cross reacted with pSCF. The result is shown as FIG. 19. The first lane with recombinant hSCF produced by E. coli is unglycosylated and has a lower molecular weight than hSCF produced in plants. The HDEL tagged proteins were preferentially retained in the endoplasmic reticulum. Because the antibody was generated against human SCF, it reacts less with porcine SCF. Hence the pSCF should be in higher concentrations than it appears. Interstitial fluid samples show the proteins were mostly degraded.

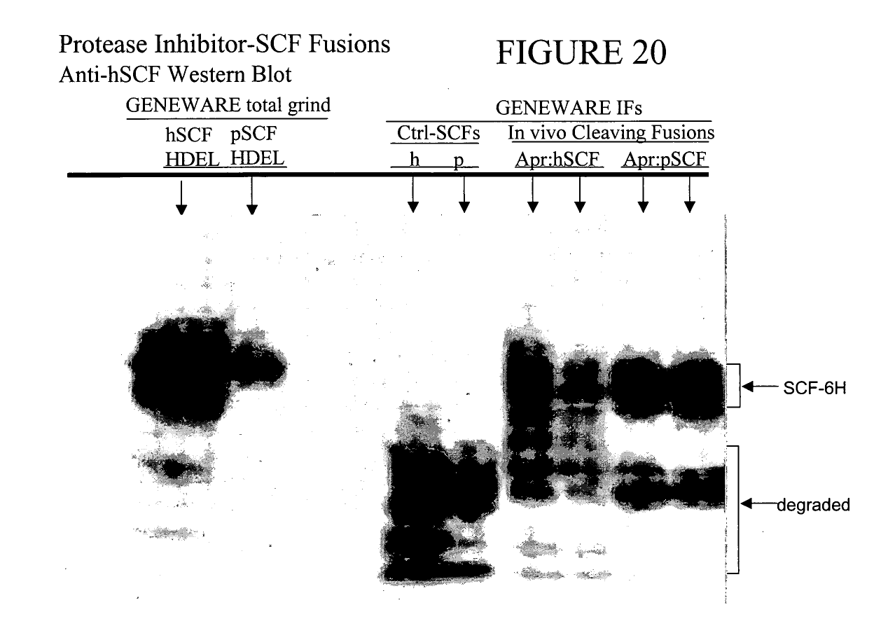

The Western blot experiment was repeated focusing on comparing the regions for both SCFs and their degradation products. This time, interstitial fluid from plants infected with GENEWARE® vectors containing aprotinin-hSCF and aprotinin-pSCF were compared to hSCF and pSCF. The key region is shown in FIG. 20. The quantity of higher molecular weight SCF protein was higher and the quantity of lower molecular weight degradation products was reduced. This suggests the presence of aprotinin reduced proteolysis compared to the same expression system without expression of aprotinin.

Although the invention has been described with reference to the presently preferred embodiments, it should be understood that various modifications can be made without departing from the spirit of the invention. All publications, patents, patent applications, and web sites are herein incorporated by reference in their entirety to the same extent as if each individual patent, patent application, or web site was specifically and individually indicated to be incorporated by reference in its entirety.

Ahiquist, P., Luckow, V., and Kaesberg, P. (1981). Complete nucleotide sequence of brome mosaic virus RNA3. J Mol Biol 153(1), 23-38.

Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res 25(17), 3389-402.

Barr, P. J. (1991). Mammalian subtilisins: the long-sought dibasic processing endoproteases. Cell 66(1), 1-3.

Barrett, A. J., and Kirschke, H. (1981). Cathepsin B, Cathepsin H, and cathepsin L. Methods Enzymol 80(Pt C), 535-61.

Batchelor, A. K., Boutilier, K., Miller, S. S., Labbe, H., Bowman, L., Hu, M., Johnson, D. A., Gijzen, M., and Miki, B. L. (2000). The seed coat-specific expression of a subtilisin-like gene, SCS1, from soybean. Planta 211(4), 484-92.

Baulcombe, D.C. (1999). Fast forward genetics based on virus-induced gene silencing. Curr Opin Plant Biol 2(2), 109-113.

Berger, D., and Altmann, T. (2000a). MEANS AND METHODS FOR MODULATING STOMATA CHARACTERISTICA IN PLANTS. Patent application WO 00/22144.

Berger, D., and Altmann, T. (2000b). A subtilisin-like serine protease involved in the regulation of stomatal density and distribution in Arabidopsis thaliana. Genes & Development 14(9), 1119-1131.

Chuang, C. F., and Meyerowitz, E. M. (2000). Specific and heritable genetic interference by double-stranded RNA in Arabidopsis thaliana. Proc Natl Acad Sci USA 97(9), 4985-90.

Creemers, J. W., Jackson, R. S., and Hutton, J. C. (1998). Molecular and cellular regulation of prohormone processing. Semin Cell Dev Biol 9(1), 3-10.

Del Mar, E. G., Largman, C., Brodrick, J. W., Fassett, M., and Geokas, M. C. (1980). Substrate specificity of human pancreatic elastase 2. Biochemistry 19(3), 468-72.

Del Mar, E. G., Larginan, C., Brodrick, J. W., and Geokas, M. C. (1979). A sensitive new substrate for chymotrypsin. Anal Biochem 99(2), 316-20.

Deom, C. M., Oliver, M. J., and Beachy, R. N. (1987). The 30-Kilodalton Gene Product of Tobacco Mosaic Virus Potentiates Virus Movement. Science 237, 389-394.

Dijkstra (1998). “Practical Plant Virology: Protocols and Exercises.” Springer Verlag.

Donald, R. G., and Jackson, A. O. (1994). The barley stripe mosaic virus gamma b gene encodes a multifunctional cysteine-rich protein that affects pathogenesis. Plant Cell 6(11), 1593-606.

Donson, J., Gunn, H. V., Woolston, C. J., Pinner, M. S., Boulton, M. I., Mullineaux, P. M., and Davies, J. W. (1988). Agrobacterium-mediated infectivity of cloned digitaria streak virus DNA. Virology 162(1), 248-50.

Elmer, J. S. (1988). Agrobacterium-Mediated Inoculation of Plants with Tomato Golden Mosaic Virus DNA's. Plant Molecular Biology 10, 225-234.

Erlanger, B., Kokowsky, N., and Cohen, W. (1961). The preparation and properties of two new chromogenic substrates of trypsin. Arch Biochem Biophys 95, 271-278.

Foster, G. D., and Taylor, S. C. (1998). “Plant virology protocols: from virus isolation to transgenic resistance.” Humana Press, Totowa, N.J.

Fukuda (1980). The Site of Initiation of Rod Assembly on the RNA of a Tomato and a Cowpea Strain of Tobacco Mosaic Virus. Virology 101, 492-502.

Gardiner (1988). Genetic Analysis of Tomato Golden Mosaic Virus: the Coat Protein is Not Required for Systematic Spread of Symptom Development. The EMBO Journal 7, 899-904.

Gardner (1986). Potato Spindle Tuber Viroid Infections Mediated by the Ti Plasmid of Agrobacterium Tumefaciens. Plant Mol. Biol 6, 221-228.

Gellerfors, P., Pavlu, B., Axelsson, K., Nyhlen, C., and Johansson, S. (1990). Separation and identification of growth hormone variants with high performance liquid chromatography techniques. Acta Paediatr Scand Suppl 370, 93-100.

Gensberg, K., Jan, S., and Matthews, G. M. (1998). Subtilisin-related serine proteases in the mammalian constitutive secretory pathway. Semin Cell Dev Biol 9(1), 11-7.

Grimsley (1986). ‘Agroinfection,’ and Alternative Route for Viral Infection of Plants by Using the Ti Plasmid. Proc. Natl. Acad. Sci. USA 83, 3282-3286.

Grimsley (1987). Agrobacterium-Mediated Delivery of Infectious Maize Streak Virus into Maize Plants. Nature 325, 177-179.

Hayes (1988). Agroinfection of Triticum aestivum with Cloned DNA of Wheat Dwarf Virus. Journal of General Virology 69, 891-896.

Hull, R. (2002). “Matthews' Plant Virology.” 4th ed. Academic Press, San Diego.

Jorda, L., Coego, A., Conejero, V., and Vera, P. (1999). A genomic cluster containing four differentially regulated subtilisin-like processing protease genes is in tomato plants. J Biol Chem 274(4), 2360-5.

Jorda, L., Conejero, V., and Vera, P. (2000). Characterization of P69E and P69F, two differentially regulated genes encoding new members of the subtilisin-like proteinase family from tomato plants. Plant Physiology 122(1), 67-73.

Kaneda, M., Yonezawa, H., and Uchikoba, T. (1995). Improved isolation, stability and substrate specificity of cucumisin, a plant serine endopeptidase. Biotechnol Appl Biochem 22(Pt 2), 215-22.

Krieg, P. (1996). “A Laboratory Guide to RNA: Isolation, Analysis and Synthesis.” Wiley-Liss, Inc, New York.

Kumagai, M. H., Donson, J., della-Cioppa, G., Harvey, D., Hanley, K., and Grill, L. K. (1995). Cytoplasmic inhibition of carotenoid biosynthesis with virus-derived RNA. Proc Natl Acad Sci USA 92(5), 1679-1683.

Kurisu, M., Ohno, T., Okada, Y., and Nozu, Y. (1976). Biochemical characterization of cucumber green mottle mosaic virus ribonucleic acid. Virology 70(1), 214-6.

Laemmli, U. K. (1970). Cleavage of structural proteins during the assembly of the head of bacteriophage T4. Nature 227(259), 680-5.

Largman, C., DelMar, E. G., Brodrick, J. W., Fassett, M., and Geokas, M. C. (1980). Inhibition of human pancreatic elastase 2 by peptide chloromethyl ketones. Biochim Biophys Acta 614(1), 113-20.

Lazarowitz, S. G. (1988). Infectivity and complete nucleotide sequence of the genome of a South African isolate of maize streak virus. Nucleic Acids Res 16(1), 229-49.

Lebel, E., Heifetz, P., Thome, L., Uknes, S., Ryals, J., and Ward, E. (1998). Functional analysis of regulatory sequences controlling PR-1 gene expression in Arabidopsis. Plant J 16(2), 223-33.

Lebeurier, G., Nicolaieff, A., and Richards, K. E. (1977). Inside-out model for self-assembly of tobacco mosaic virus. Proc Natl Acad Sci U S A 74(1), 149-53.

Lindbo, J. A., Fitzmaurice, W. P., and della-Cioppa, G. (2001). Virus-mediated reprogramming of gene expression in plants. Curr Opin Plant Biol 4(3), 181-185.

MacFarlane, S. A., and Popovich, A. H. (2000). Efficient expression of foreign proteins in roots from tobravirus vectors. Virology 267(1), 29-35.

Martinez, A., Sparks, C., Drayton, P., Thompson, J., Greenland, A., and Jepson, I. (1999). Creation of ecdysone receptor chimeras in plants for controlled regulation of gene expression. Mol Gen Genet 261(3), 546-52.

McCormick, A. A., Kumagai, M. H., Hanley, K., Turpen, T. H., Hakim, I., Grill, L. K., Tuse, D., Levy, S., and Levy, R. (1999). Rapid production of specific vaccines for lymphoma by expression of the tumor-derived single-chain Fv epitopes in tobacco plants. Proc Natl Acad Sci USA 96(2), 703-708.

Meichtry, J., Amrhein, N., and Schaller, A. (1999). Characterization of the subtilase gene family in tomato (Lycopersicon esculentum Mill.). Plant Mol Biol 39(4), 749-60.

Meshi (1983). Nucleotide Sequence of the Coat Protein Cistron and the 3′ Noncoding Region of cucumber Green Mottle Mosaic Virus (Watermelon Strain) RNA. Virology 127, 54-64.

Mett, V. L., Lochhead, L. P., and Reynolds, P. H. (1993). Copper-controllable gene expression system for whole plants. Proc Natl Acad Sci USA 90(10), 4567-71.

Miller, J. (1984). Virology 132, 71.

Moss, D. E., and Fahrney, D. (1978). Kinetic analysis of differences in brain acetylcholinesterase from fish or mammalian sources. Biochem Pharmacol 27(23), 2693-8.

Nagar, S., Pedersen, T. J., Carrick, K. M., Hanley-Bowdoin, L., and Robertson, D. (1995). A geminivirus induces expression of a host DNA synthesis protein in terminally differentiated plant cells. Plant Cell 7(6), 705-19.

Nakajima, K., Powers, J. C., Ashe, B. M., and Zimmerman, M. (1979). Mapping the extended substrate binding site of cathepsin G and human leukocyte elastase. Studies with peptide substrates related to the alpha 1-protease inhibitor reactive site. J Biol Chem 254(10), 4027-32.

Neurath, H. (1984). Evolution of proteolytic enzymes. Science 224(4647), 350-7.

Neuteboom, L. W., Ng, J. M., Kuyper, M., Clijdesdale, 0. R., Hooykaas, P. J., and van der Zaal, B. J. (1999). Isolation and characterization of cDNA clones corresponding with mRNAs that accumulate during auxin-induced lateral root formation. Plant Mol Biol 39(2), 273-87.

Nishikura, K. (2001). A short primer on RNAi: RNA-directed RNA polymerase acts as a key catalyst. Cell 107(4), 415-8.

Nozu, Y., Tochihara, H., Komuro, Y., and Okada, Y. (1971). Chemical and immunological characterization of cucumber green mottle mosaic virus (watermelon strain) protein. Virology 45(3), 577-85.

Old, and Primrose (1994). “Principles of Gene Manipulation.” 5th ed ed. Blackwell Science, Oxford, U.K.

Petty, I. T., Hunter, B. G., Wei, N., and Jackson, A. O. (1989). Infectious barley stripe mosaic virus RNA transcribed in vitro from full-length genomic cDNA clones. Virology 171(2), 342-9.

Plunkett, G., Senear, D. F., Zuroske, G., and Ryan, C. A. (1982). Proteinase inhibitors I and II from leaves of wounded tomato plants: purification and properties. Arch Biochem Biophys 213(2), 463-72.

Ratcliff, F., Martin-Hemandez, A. M., and Baulcombe, D. C. (2001). Tobacco rattle virus as a vector for analysis of gene function by silencing. Plant Journal 25(2), 237-245.

Ratcliff, F. G., MacFarlane, S. A., and Baulcombe, D. C. (1999). Gene silencing without DNA: RNA-mediated cross-protection between viruses. Plant Cell 11(7), 1207-1215.

Ribeiro, A., Akkermans, A. D., van Kammen, A., Bisseling, T., and Pawlowski, K. (1995). A nodule-specific gene encoding a subtilisin-like protease is expressed in early stages of actinorhizal nodule development. Plant Cell 7(6), 785-94.

Rudenskaya, G. N., Bogacheva, A. M., Preusser, A., Kuznetsova, A. V., Dunaevsky Ya, E., Golovkin, B. N., and Stepanov, V. M. (1998). Taraxalisin—a serine proteinase from dandelion Taraxacum officinale Webb s.1. FEBS Lett 437(3), 237-40.

Rudenskaya, G. N., Bogdanova, E. A., Revina, L. P., Golovkin, B. N., and Stepanov, V. M. (1995). Macluralisin—a serine proteinase from fruits of Maclura pomifera (Raf.) Schneid. Planta 196(1), 174-9.

Salem, M. A. (1988). Effects of the amino-terminal portion of human growth hormone on glucose clearance and metabolism in normal, diabetic, hypophysectomized, and diabetic-hypophysectomized rats. Endocrinology 123(3), 1565-76.

Siekierka, J. J., Hung, S. H., Poe, M., Lin, C. S., and Sigal, N. H. (1989). A cytosolic binding protein for the immunosuppressant FK506 has peptidyl-prolyl isomerase activity but is distinct from cyclophilin. Nature 341(6244), 755-7.

Siezen, R. J., and Leunissen, J. A. (1997). Subtilases: the superfamily of subtilisin-like serine proteases. Protein Sci 6(3), 501-23.

Sinha, Y. N., and Jacobsen, B. P. (1994). Human growth hormone (hGH)-(44-191), a reportedly diabetogenic fragment of hGH, circulates in human blood: measurement by radioimmunoassay. J Clin Endocrinol Metab 78(6), 1411-8.

Steiner, D. F., Smeekens, S. P., Ohagi, S., and Chan, S. J. (1992). The new enzymology of precursor processing endoproteases. J Biol Chem 267(33), 23435-8.

Tanaka, H., Onouchi, H., Kondo, M., Hara-Nishimura, I., Nishimura, M., Machida, C., and Machida, Y. (2001). A subtilisin-like serine protease is required for epidermal surface formation in Arabidopsis embryos and juvenile plants. Development 128(23), 4681-4689.

Taylor, A. A., Horsch, A., Rzepczyk, A., Hasenkampf, C. A., and Riggs, C. D. (1997). Maturation and secretion of a serine proteinase is associated with events of late microsporogenesis. Plant J 12(6), 1261-71.

Tomero, P., Conejero, V., and Vera, P. (1996). Primary structure and expression of a pathogen-induced protease (PR-P69) in tomato plants: Similarity of functional domains to subtilisin-like endoproteases. Proc Natl Acad Sci U S A 93(13), 6332-7.

Tomero, P., Conejero, V., and Vera, P. (1997). Identification of a new pathogen-induced member of the subtilisin-like processing protease family from plants. J Biol Chem 272(22), 14412-9.

Twining, S. S. (1984). Fluorescein isotbiocyanate-labeled casein assay for proteolytic enzymes. Anal Biochem 143(1), 30-4.

Uchikoba, T., Yonezawa, H., and Kaneda, M. (1995). Cleavage specificity of cucumisin, a plant serine protease. J Biochem (Tokyo) 117(5), 1126-30.

Umezawa, H. (1976). Structures and activities of protease inhibitors of microbial origin. Methods Enzymol 45, 678-95.

Waterhouse, P. M., Graham, M. W., and Wang, M. B. (1998). Virus resistance and gene silencing in plants can be induced by simultaneous expression of sense and antisense RNA. Proc Natl Acad Sci US A 95(23), 13959-64.

Weiland, J. J., and Edwards, M. C. (1996). A single nucleotide substitution in the alpha a gene confers oat pathogenicity to barley stripe mosaic virus strain ND18. Mol Plant Microbe Interact 9(1), 62-7.

Wilson, T. M. A., and Hwang-Lee, D.-J. (1995). RNA packaging system. U.S. Pat. No. 5,443,969.

Wroblewski, V. J., Kaiser, R. E., and Becker, G. W. (1993). Proteolysis of human growth hormone by rat thyroid gland in vitro: application of electrospray mass spectrometry and N-terminal sequencing to elucidate a metabolic pathway. Pharm Res 10(8), 1106-14.

Yamagata, H., Masuzawa, T., Nagaoka, Y., Ohnishi, T., and Iwasaki, T. (1994). Cucumisin, a serine protease from melon fruits, shares structural homology with subtilisin and is generated from a large precursor. J Biol Chem 269(52), 32725-31.

Yamagata, H., Uesugi, M., Saka, K., Iwasaki, T., and Aizono, Y. (2000). Molecular cloning and characterization of a cDNA and a gene for subtilisin-like serine proteases from rice (Oryza sativa L.) and Arabidopsis thaliana. Bioscience Biotechnology and Biochemistry 64(9), 1947-1957.

What is claimed is:

1. A host cell comprising one or more polynucleotides, wherein said one or more polynucleotides encode a protein of interest and a genetic element capable of reducing a protease activity in a host cell or fluids, wherein said one or more polynucleotides is capable of expressing said protein of interest in the host cell, wherein said protease activity is capable of cleaving said protein of interest, wherein said protein of interest is non-native to the host cell.

2. The host cell according to claim 1, wherein said genetic element (1) expresses an antisense or sense element capable of reducing expression of a protein with said protease activity in said host cell, (2) expresses a ribozyme capable of reducing expression of a protein with said protease activity in said host cell, (3) induces expression of a protease inhibitor native to said host cell in said host cell, or (4) expresses a protease inhibitor in said host cell.

3. The host cell according to claim 2, wherein said genetic element expresses an antisense or sense element capable of reducing expression of a protein with said protease activity in said host cell.

4. The host cell according to claim 3, wherein said antisense or sense element comprises a nucleotide sequence that is substantially similar to the antisense or sense nucleotide sequence of a protease.

5. The host cell according to claim 4, wherein said antisense or sense element comprises the antisense or sense nucleotide sequence of said protease.

6. The host cell according to claim 5, wherein said host cell is a plant cell.

7. The host cell according to claim 5, wherein said protease is native to said host cell.

8. The host cell according to claim 5, wherein said protease is a serine protease.

9. The host cell according to claim 8, wherein said serine protease is a chymotrypsin-like serine protease or a subtilisin-like serine protease.

10. The host cell according to claim 9, wherein said subtilisin-like serine protease is a Nicotianalisin protein and the host is a plant cell.

11. The host cell according to claim 1, wherein said protein of interest is a protein not native to the host cell.

12. The host cell according to claim 11, wherein said protein is a human protein.

13. The host cell according to claim 12, wherein said human protein is human growth hormone.

14. The host cell according to claim 1, wherein said first polynucleotide is non-native to said host cell.

15. The host cell according to claim 14, wherein said polynucleotide encoding the protein of interest inserted into a viral vector.

16. The host cell according to claim 15, wherein said viral vector is obtained from a RNA virus.

17. The host cell according to claim 11, wherein said host cell is a plant cell.

18. The plant cell according to claim 17, wherein said one or more polynucleotides is in a vector.

19. The plant cell according to claim 17, wherein the polynucleotide encoding a protein of interest and/or the polynucleotide encoding a genetic element capable of reducing a protease activity is integrated into the plant genome.

20. The host cell according to claim 1, wherein said polynucleotide encoding the genetic element is inserted into a viral vector.

21. The host cell according to claim 1, wherein said genetic element encodes a protease inhibitor.

22. The host cell according to claim 21, wherein said protease inhibitor is aprotinin.

23. The host cell according to claim 21, wherein said genetic element and said polynucleotide encoding a protein of interest are both inserted into a vector.

24. The plant cell according to claim 23, wherein said polynucleotides encoding said genetic element and said protein of interest are fused together to produce a fused protein product.

25 The plant cell comprising the plant cell according to claim 6.

26. A plant comprising the plant cell according to claim 17.

27. A polynucleotide comprising a substantially similar or complementary sequence of at least a part of the coding sequence, or one or more fragments, of a Nicotianalisin protease which is not identical with another protease.

28. A method of reducing the amount of a protein of interest cleaved by a hydrolase activity in a host cell, comprising the steps of:
(a) introducing a polynucleotide into a host cell, wherein said polynucleotide comprises a genetic element capable of reducing a hydrolase activity in said host cell; and
(b) expressing a protein of interest in said host cell, wherein said protein of interest is capable of expression in said host cell, wherein said protein of interest is capable of being cleaved by said hydrolase activity; whereby the amount of protein of interest cleaved by said hydrolase activity in said host cell is reduced compared to the amount of protein of interest cleaved by said hydrolase activity in another host cell in which said polynucleotide is not introduced.

29. The method of claim 28 wherein said protein of interest is heterologous to said host.

30. The method according to claim 28, further comprising the step of isolating the protein of interest from said host cell or fluid.

31. The method according to claim 28 wherein said host cell is a plant cell.

32. The method of claim 29 wherein said polynucleotide is in a vector.

33. The method of claim 29 wherein said hydrolase is a protease.

34. A vector containing a genetic element capable of reducing protease activity and a polynucleotide encoding a protein of interest.

35. A composition of purified Nicotianalisin having a specific activity greater than 100 units/mg protein.

36. The composition of claim 34 wherein Nicotianalisin is substantially isolated from other proteins and having an activity greater than 3000 units/mg protein.

37. A method for cleaving a polypeptide comprising contacting a composition containing the Nicotianalisin of claim 35 with a polypeptide substrate for a time and under conditions sufficient to cleave the polypeptide.


SEQUENCE LISTING (40 sequences)

SEQ ID NO: 1 - DNA, 39 bp, Primer
gtgagggcaagacattgatgtgcctgatatgatattgaa

SEQ ID NO: 2 - DNA, 24 bp, Primer
ggcgtgattatcggagttatagac

SEQ ID NO: 3 - DNA, 1194 bp, Artificial Sequence
  TMV Vector Construct
ggcgtgattatcggagttatagacactggaattttccctgaccatccttcatttagcgacgttgggatgtctcccccgcctgctaagtggaaaggattttgtgagtccaatttcacgacaaagtgtaataacaagatcataggactcaggtcttttcgattatctgaagataccccgatagatactgatggacatggtacacacactgctagcacagctgcaggagcttttgtgaaaggtgccaatttctttggtaatgcaaatggcacagcagttggtgttgcccctcttgcccacatggccatatataaggtatgcagttttgctacttgtagtgaaactgatgctttagctgccatggatgcagctatagatgatggtgtagatatcatttccgcatctctaggcggatttactaacgctccattacatgatgaccctatttctcttggcgcgtacagtgcaacagaaaaaggtattctngctagtgcctctgcaggcaatagcgagtttgataaccctgtagcaaataatgccccttggattctcacagttggcgctagcacccatgatagaaaactaaaagccaccgttaagcttggaaataaagaggaatttgaaggagaatctgctgatcagccaaagacttccaactcaacattcatcgctctatttgatgctggaaagaatgcaagtgatcaagatgctccattctgtagatcgtgggcgatgactgatcctgctatcaaaggtaagatagtcttgtgtcaaaaagacccaagtagtctcaccagtagtcaaggacgaaatgtaaaggacgctggaggcgttggcatgattctcatcaataatccggaagatggtgtcactaaatcagctactgctcatgttcttcccgcattagatgtttcacatgaagaaggagagaaaattaaggcctatataaattcaacttcaaatcctattgctgcaattacattccagggaacagtaataggagataaaaatgctcctattgttgcttcattttctgctcgaggaccaagccgagcaaaccctggcatcttaaaacctgatattattggtcctggtgttaatatccttgctgcttggcctaccaccgtgaatatccccaacaaaaacacaaattctggattcaatatcatatcaggcacatcaatgtcttgccctcac

SEQ ID NO: 4 - DNA, 1194 bp, Artificial Sequence
  TMV Vector Construct
ggcgtgattatcggagttatagacactggaattgttcctgaccatccttcatttagcgacgttgggatgcctcctccgcctgctaaatggaaaggattttgtgagtctaatttcacgaccaagcgtaacaacaaactcattggagccaggtctttcccgcttgacaatggtcccatagatgaaaatggacatggtacgcatacagcaagcacagctgcaggagcctttgtgaaaggtgctaatgtatttgggaatgccaatggaacagcagttggtgttgcccctcttgcgcacatagccatatataaggtatgcggttctgatggcgtttgttctgatgttgaaattttacctgcgatggatgtagctattgatgatggcgtagatattctatcaatatcccttggtggaactagtaatccgttccataatgacaagattgctcttggggcgtatagtgcaacagaaagaggtattcttgttagttgttctgcaggcaatagtggtccattccaacgcactgtaaacaatgacgccccttggattctcacagttggcgctagcactcatgatagaaaactaaaggccactgttaagcttggaaataaagaagaatttgaaggagaatctgcttatcatccaaagacttcaagctcaacattcttcactctatttgatgttgaaaaagatggtacacgagcaaccagagcccctttctgcataccaggatcactcactgacccttctataaggggaaagatagttgtgtgcctggttggtggtggcgttcgtacggttgataaaggacaagttgtaaaggatgctggaggtgttggcatgattcttatcaataatccagaagatggtgttactaaatcagctgaagctcatgtccttccagcattggatgtttcagatgcagatggaaagaaaattcttgcctacataaactcaacgtcgaatcctgttgctgcaatcaccttccatggaactgtacttggagataaaaatgctcctatagttgcttcattttcttctcgaggaccaagcgaagcaagtcgtggcatcttgaaacctgatattattggtcctggtgttaatgtccttgctgcttggcctacctcagtagataacaacaaaaacacaaaatccacattcaatatcatatcaggcacatcaatgtcttgccctcac

SEQ ID NO: 5 - DNA, 2533 bp, Artificial Sequence
  Expresses homologous to  A thaliana, BAB02339 cucumisin-like serine protease
cccacgcgtccggtttttccattctttttcattattatctctttctgcctcactccggtgaccatttccgtccaatccgatggtcatgaaactttcatcattcacgtttccaaatccgataagccccgtgttttcaccacccaccaccattggtactcctccatcatccgatccgtttcccaacacccttctaaaatcctctacacctatgaacgtgccgccgtgggcttctctgcacgcctcacagccgctcaggccgatcagctccgccgtattcccggtgtaatctccgtccttcccgacgaagtacgccatctccacaccacccatacccctaccttcttgggacttgctgactctttcggcctttggcccaactccgattacgctgatgatgtcatcgtcggagttctggacacgggtatatggccggaaagaccgagtttttccgacgagggtctctctacggttccttcaagttggaaagggaagtgcgttactggacctgattttcctgaaacctcatgtaataaaaaaatcataggcgctcaaatgttttacaaagggtatgaagctaaacatggcccaatggatgaatcaaaagaatcaaaatcgccaagagatactgaaggacatggaacacatacagcatcaacagcagctggttctttagtggcaaatgctagcttttaccaatatgccaaaggtatggctataaaagcaagaatagccgcttacaagatttgctggaaaaatggctgttttaattctgatatattggctgccatggatcaagctgttgatgatggtgtgcatgtgatctcactttctgttggggctaacggttatgctccacattatctgtatgattctattgcaattggagcttttggtgcatctgaacatggcgtcctcgtctcatgttcagctggaaattctggtcccggagcttatacggcagtgaacattgccccgtggatgctcactgttggtgcatcaactatagatcgtgagttcccggcagatgttattttaggagataatagaatatttggtggtgtttcattgtactccggcaatcctttgaccgatgccaaattgccggtggtttattccggcgactgtggtagcaaatactgttatccaggaaagctagatcctaaaaaagtcgcaggaaaaattgttttatgcgataggggaggcaacgctagggttgaaaaagggagtgcggtgaagcaggcaggcggagtagggatgatacttgctaatttggctgactccggcgaagaactcgtcgccgattcacatcttctcccggcgacgatggtcggtcaaaaagctggagacaaaataagacactacgtaacgtctgatccttcacccacggcgacgatcgtgttcagaggaacagtgatcggaaaatcaccggcagcaccacgtgtagcggcgttctcgagccgaggacctaatcatttgacgccggagattcttaaaccggatgttattgcacctggagttaacattttggccggttggaccggatctgttggaccgaccgatttggatattgacacgagaagagtagaattcaatattatttctggaacttccatgtcgtgccctcacgttgggggattggctgctttacttagaagggcccacccaaagtggaccccagcagcggtaaagtcagcacttatgacaacagcttacaacttggacaattctggtaaagtatttacagatcttgccactggccaagaatctactcccttcgttcatggatcaggtcatgtagacccgaaccgagcattggatccgggtttgatttacgacatcgaaactagcgattacgtaaatttcctatgctccatggcttatgacggcgacgatgtcgccgtgttcgcgagagattcttctcgagtgaattgcagtgaacgaagtttggctactccgggagacctgaattacccgtcgttctccgttgtttttaccggtgagagcaacggtgtggttaaatacaagcgggtggtgaataatgtaggaaaaaatacagatgctgtgtatgaagtgaaggtgaatgcgccgtcgtcggtggaggtgaatgtatcaccggcgaagcttgtattcagtgaggaaaagcaaagtttgtcgtatgagattagcttaaagagtaaaaagagtggtgatttgcagatggtgaaggggattgaatctgcatttgggtcgattgaatggagtgatggaattcacaatgtgagaagcccaattgcggtgcgttggcgtcactattctgatgcagcatccatgtgagtaatggatgattgttctttatattgcattgcatggaccaataaactgggatgatgacaaattgaaagacgaaatgttgctagaggatcatcgaatttgtccaactttaatttcactttctttaccttttgttctctgatgttgttcagattgatgtatatatgaatgaagcatacccagttgtttcccagaaaaaaaaaaaaaaaaaaaaaaaaaaaaa

SEQ ID NO: 6 - DNA, 813 bp, Artificial Sequence
  Clone that expresses homologous to A thaliana, AAF76468 similar to p69d gene of tomato
ggccattacggccgggcacgtgaatcctgaatcggctattgatccgggcctaatatacgacactgatacatcagactacatcaacctactatgcagcttgaactacacagagaaagaaatgaaacttttcacgaacgagtcaaatccttgctcgggttttactggatctccacttgatcttaactatccatcactttctgttatgttcaggcctgattcctctgttcatgttgttaagaggacattaacacatgtcgcggtttctaagcctgaggtgtacaaagtaaagatactgaatctgaattctgaaaaggttagtttaagtataagcccaatggagctgatgttcaatgaatctttaaggaaacaaaggtatatggtcaaatttgagagccatcatatattcaacagcagcaggaaaatagctgagcaaatggcgtttggttcgatatcttgggagagtgaaaagcacaatgttaggagcccctttgctgttatgtgggttcagcaaaatttcaataacagtagattatacaaaataacttaatatgtacattgttgtatctgttgtatcgtcgtacctctagcctccgagtatgtactatgttgtattacgtacctctagcctctgagtatgtactatgttgtatcatcgtacctctagcctccgagtatgtactacgttgtattacgtacctctagcctctgagtatgtactatgttgtatcaacgtacctctagcctccgagtatgtagtatgtatatcattgtatctttaggcctcaaaaaaaaaaaaaaaaaaaaaaaaaaaaa

SEQ ID NO: 7 - DNA, 869 bp, Artificial Sequence
  Expresses homologous to Alnus glutinosa, S52769 subtilisin-like proteinase ag12
tggtatcaacgcagagtgccattacggccggggatgatgggattagtgaagtaccatcaagatggaaaggagaatgtgaaagtggtactgagttcaattcctctttgtgtaacaagaagctcattggcgctcgttacttcaataaaggcctacttgccaacaatccaaatcttaatatttcaatgaattcttctagagataccgatggacatggaactcacacttcttctacagctgcgggaagttatgttgagggtgcatcttattttggctatgccaccggtactgctattggcatagcgccaaaggctcatgtggctatgtacaaggctctatgggaagaaggtgtatacttgtctgatgttcttgctgcaattgatcaagcaattacagatggtgtggatgtcttatccttgtcgttaggcatagacgcgattccactacacgaagatcctgtggcaattgccgcatttgctgcattggagaaaggtatatttgtttccacctctgcaggaaatgaagggccttattatgagactttgcacaatggaacaccttgggtgttaactgttgcagctggcacagttgaccgcgaatttattggaacattaactcttggaaatggagtttcagtccctggtttatcgctataccctgggaattctagttcaagcgaaagctcccttgtctatgtcgaatgccaagatgacaaggaactgcagaaaaatgcacacaaatttgttgtctgtctcgacaagaatgattcggttggtgagcatgtgtacaatgtaagaaattcaaaagttgctggggctgtctttataactaatacaactgacttggaattctacctccaaagcgaattcccg

SEQ ID NO: 8 - DNA, 2532 bp, Artificial Sequence
  Expresses homologous to A thaliana, BAB02339 cucumisin-like serine protease
gtttttccattctttttcattattatctctttctgcctcactccggtgaccatttccgtccaatccgatggtcatgaaactttcatcattcacgtttccaaatccgataagccccgtgttttcaccacccaccaccattggtactcctccatcatccgatccgtttcccaacacccttctaaaatcctctacacctatgaacgtgccgccgtgggcttctctgcacgcctcacagccgctcaggccgatcagctccgccgtattcccggtgtaatctccgtccttcccgacgaagtacgccatctccacaccacccatacccctaccttcttgggacttgctgactctttcggcctttggcccaactccgattacgctgatgatgtcatcgtcggagttctggacacgggtatatggccggaaagaccgagtttttccgacgagggtctctctacggttccttcaagttggaaagggaagtgcgttactggacctgattttcctgaaacctcatgtaataaaaaaatcataggcgctcaaatgttttacaaagggtatgaagctaaacatggcccaatggatgaatcaaaagaatcaaaatcgccaagagatactgaaggacatggaacamatacagcatcaacagcagctggttctttagtggcaaatgctagcttttaccaatatgccaaaggtgaagctagaggtatggctataaaagcaagaatagccgcttacaagatttgctggaaaaatggctgttttaattctgatatattggctgccatggatcaagctgttgatgatggtgtgcatgtgatctcactttctgttggggctaacggttatgctccacattatctgtatgattctattgcaattggagcttttggtgcatctgaacatggcgtcctcgtctcatgttcagctggaaattctggtcccggagcttatacggcagtgaacattgccccgtggatgctcactgttggtgcatcaactatagatcgtgagttcccggcagatgttattttaggagataatagaatatttggtggtgtttcattgtactccggcaatcctttgaccgatgccaaattgccggtggtttattccggcgactgtggtagcaaatactgttatccaggaaagctagatcctaaaaaagtcgcaggaaaaattgttttatgcgataggggaggcaacgctagggttgaaaaagggagtgcggtgaagcaggcaggcggagtagggatgatacttgctaatttggctgactccggcgaagaactcgtcgccgattcacatcttctcccggcgacgatggtcggtcaaaaagctggagacaaaataagacactacgtaacgtctgatccttcacccacggcgacgatcgtgttcagaggaacagtgatcggaaaatcaccggcagcaccacgtgtagcggcgttctcgagccgaggacctaatcatttgacgccggagattcttaaaccggatgttattgcacctggagttaacattttggccggttggaccggatctgttggaccgaccgatttggatattgacacgagaagagtagaattcaatattatttctggaacttccatgtcgtgccctcacgttgggggattggctgctttacttagaagggcccacccaaagtggaccccagcagcggtaaagtcagcacttatgacaacagcttacaacttggacaattctggtaaagtatttacagatcttgccactggccaagaatctactcccttcgttcatggatcaggtcatgtagacccgaaccgagcattggatccgggtttgatttacgacatcgaaactagcgattacgtaaatttcctatgctccattggctatgacggcgacgatgtcgccgtgttcgcgagagattcttctcgagtgaattgcagtgaacgaagtttggctactccgggagacctgaattacccgtcgttctccgttgtttttaccggtgagagcaacggtgtggttaaatacaagcgggtggtgaataatgtaggaaaaaatacagatgctgtgtatgaagtgaaggtgaatgcgccgtcgtcggtggaggtgaatgtatcaccggcgaagcttgtattcagtgaggaaaagcaaagtttgtcgtatgagattagcttaaagagtaaaaagagtggtgatttgcagatggtgaaggggattgaatctgcatttgggtcgattgaatggagtgatggaattcacaatgtgagaagcccaattgcggtgcgttggcgtcactattctgatgcagcatccatgtgagtaatggatgattgttctttatattgcattgcatggaccaataaactgggatgatgacaaattgaaagacgaaatgttgctagaggatcatcgaatttgtccaactttaatttcactttctttaccttttgttctctgatgttgttcagattgatgtatatatgaatgaagcatacccagttgtttcacagaaaaaaaaaaaaaaaaaaaaaaaaaaaa

SEQ ID NO: 9 - DNA, 716 bp, Artificial Sequence
  Expression homologous to Tomato, T07172 subtilisin-like proteinase
cccacgcgtccgatggtgcaggacacataaatccacgaaaagctgttgaccctggtttggtttatgacataggggcgcaggattacttcgaattcctatgcacacaacaactcagcccttcacagctaacagtttttggaaagttctccaacagaacttgccatcactcgcttgctaatccaggggacttgaactaccccgccatttctgcagtttttcctgaagacgcaaaagtttcaacgctgacgcttcacagaacagtcaccaatgtgggttctccaatatcaaattaccatgttagagtctcgccgttcaaaggtgcagtcgtgaaggttgagccatcaagattgaatttcaccagcaaacaccagaaactatcttacaaggtgattttcgagacaaaatatcgtcaaaaagcacgtgaatttggatccctgctttggaaagatgggacacacaaagtgagaagcacaattgtgatcacatggctagcatcaatttagttcagcttgccatttagtaactagcattttgtttgctgaggataataaaaaacatgttgcggcaaagtccctgtgttttcagcagttgttttcagtagaaaatagtttcttttgttcattactagctacttatattgccaaattatttgtggcagctactaaaatgatgaatttggcaataggagccaaaaaaaaaaaaaaagggcggccgcg

SEQ ID NO: 10 - DNA, 2691 bp, Artificial Sequence
  Expression homologous to Tomato, T07171 subtilisin-like proteinase SBT1
gctagcacccaaccccctctctaccaaaggaaagaaactcttccctttcagtagagaacttccattttctggttacctgacactgagaaccgcaaaaagatggcaagacccgggggcatggttctttcaacactattcctaatgttgtttcatgtgtttgttcatgcaggccagaaccagaaaaagacttacataatttacatggacaagtccaacatacctgctgattttgatgatcacactctgtggtatgactcatctttaaagtcagtatccaaaggcgccaacatgctttacacctacaacaatgtcatccatggctactcaacacagctaacagctgatgaagccaaatctcttgaacagcaacctggaattctctcggtccatgaggaagtgagatacgagcttcataccactcgatcccctacatttctgggacttgaaggacgtgaaagtaaatcattctttcttcaagctgaaacaaggagtgaggtcattattggtgtgctggacactggtgtttggcctgaatcaaaaagttttgatgacactggactaggtccagtccctatgagctggaagggtgagtgccaaattggcaagaacttcaaagcatcaagctgtaaccggaaactcattggtgcaaggtttttctcacaaggttatgaagcagctttcggggcaattgatgagaccacagaatccaagtcaccaagggacgatgatggccatgggacacacactgcaactacagcagctggctcggttgtaacgggagctagcctctttggttatgctgctggcacagcacgtgggatggcttcacatgcaagagtggctgcatataaagtatgttgggctggaggatgttttagcagcgacatactagcagggatggatcaggccgtcatagatggtgtaaatgtactctcactgtcccttggtggcacaatttctgattattacagggatatagtagcaattggaggattttctgcagcttctcaaggaatcttcgtctcgtgctcggctgggaatggcgggccaggctctggatccctctccaacgctgcaccatggataactactgtaggtgcggggaccatggaccgcgaattcccagcatatattagccttggaaatggaaaaaaattcagtggagtatcactttacagtggaaaagcattacctagttctgtgatgccactggtgtatgctggaaatgccagccaagcatcaaatggcaatttatgcacaagtggtagtctgattccagaaaaagttgatgggaaaattgtagtatgtgacagagggatgaatgcaagggcacagaagggtttggttgtcaaagatgctggtggaatagggatgattttggcaaacacagactcttacggagatgagttggttgctgatgcmcatctcataccaacaggtgcagttggtcaaactgctggtganttgatcnaaaggtacattgcttctgacagtaatccaattaccacaattgcatttggaggtaccaagttgggcgtccaaccatcaccggtcgtcgcagcttttagttccagagggccaaacccaatcacaccggagatccttaaaccagatttgatagcaccaggtgtcaatattcttgctggctggacaggaaaagttggaccaacaggtttgccagaagacaccaggaatgtgggtttcaacatcatctctggaacttccatgtcatgtcnccatgtaagtgggcttgcagcantactgnaagccgcccatccagaatggagttnaggggtnataaggtcagcactgatgactacaggttacagcacacacaagaatggnnaaatgatagaggatgttgccacaggaatgtcatatacaccagttgatcatggcgctggacatgtgaatccagcagcagctatgaatcctgggttagkgtatgatctcacagttgatgactatataaacttcctttgcgccctggattacagtccaagtatgatcaaggtcatcgcaaagcgagatatttcctgcgnaaacaataaggatatagagttgctgaccttaattacccatcttttgccattcctttggaaacgggcctggggcgaacatgcaaatagtagtgcaccaacagtgaccagatatacgaggactctaacaaacgtgggaaatccagctacatacaaggcctcagtctcttctgaaatgcaggaagtgaagattcaggttgaaccacaaacacttactttcagtcgaaagaaggaaaagaaaacctacactgtgacattcactgctagttccaagccatcaggcacaactagctttgctcgactggaatggtcagatggacaacatgttgttgctagcccaattgctttcagttggacatgattatgctaattctataagtcattcactgcaaatgtacaagtgcaaatattcctataaaataattactagtgtgcagcagctactcctctaatattccaccaactaaaaaaatagccctgacctataattaagatgcctaggaaattctagcatctagacaaggaaaatgttggttgatttgtccagcaaaagacaggtgttttacttgccagattattatgtaccaagccacacaatatggataaatacaattggctttcgaaaaaaaaaaaaaaaaaaaaaaaaaaaaaa

SEQ ID NO: 11 - DNA, 2188 bp, Artificial Sequence
  Expression homologous to Alnus glutinosa, S52769 subtilisin-like proteinase ag12
acccctcgaccmacgcgycsgcccacgcgtccgcccacgcgtccgctttctccttccgagtacaaagccatcaagaattctctagggtatgtttcttcgatggaggacaggacagttaaaattcacacgacccattcatcccatttccttggcctaagctcaatgtatggttcatggccaaagtcaaactatggcaaaggtgttatcattggtgtagttgatacaggggtttggccagagattaaaagctttgatgatgatgggatgagccaagttccatcaaggtggaaaggaatatgtcaaactggcactcagtttaattcttcattgtgcaacaagaaactcatcggagctcgttacttcaataaaggactactttctaaagtgaaaaatcttaccatcatgataaattctgcccgtgatacagagggacatggaactcatacttcctctacagctgctggaagtcttgtaaagggtgcgtcttattttggctatgcccctggttttgcaataggcgtcgcaccaatggctcatgtggctgtgtacaaggctctctgggatggggccggtaccatttctgatattcttgctgcattggatcaggcaattgcggatggttgtgatatcttatccttgtcatttggcgcagtttctccattccctctatatatagatcctatctctattgcttcattttctgcaatggagaaaggcatatttgtttccgtttcagctggaaatgaagggcctttcgatcaatctttgagcaatgaggcaccttggtttctctctgttgctgctagcacagttgatcgggacgttatcaggatattaactcttggtaatggagtttcagtcactggtttatctctctaccctgggaattctacaagcgatatttctgttattcttgtcaagaattgcttagataagcaggaattgcaaaatgttacagacaaatttgtggtctgcattgacaaaaacgcattggtcgggaaacaagttgaaagtgtgagacattcaaatgctgctggtgctgtcttcataacaaatgactttgtcactgacttgggcgaatacctcaaaacagaattcccatctgtgtttctgaatttccaaaatggtgatcaagttttgaaatatgttaacagcacttcttcaccaaaagcaaagattggacttcaagggacactaattggtgtcgaacgagcaccagctgtcgcgcattttagttcgagggggccatcaatgacctgcccgtttatcctcaaacctgacctgatggctccaggtcacttaatactagcttcatggtctccactatcatctgtgagtccatatactgaacttcacaatatctttaacattatatctggcacatccatgtcatgtccacacgctgccggtgtagctgcacttgttaaagggacccaccctgaatggagcccagctgccattcgttcggccatgatgactacagcggatgttctagacaacacacaaagtccgatccaagacatcggtcgtccagagaatgctgctgctactcctcttgctatgggagctggccatatcaatcctaacaaggcaatagatcctggactcatctatgatacaacaccacaagattacattaatcttctttgtgctctaaatctcacatccgagcagataaaaaccatcactaggtcctcttatacttgccccaacccatcattggacctaaactatccatctttcattgcctatttcaacgtgaatagcagcgagttggatcctacaagagtacaagaattcaagaggacagtgactaatgtcggagaaggtgtgtcggaatatacagccgagctgactgcaatgcctggacttaaagttagtgttgttcctgaaaagttggttttcaaagacaagtatgaaaagcaaagctacaagctgaggatagaatgtccacaactgatgaatgatttcttggttcatggttctttaagctgggtggaaaagggaggtaaatatgtagttaggagcccaattgttgccacaaattcttaagtttgatcctttgacaggatagtactgattactgaatattccactaaacatgtcttttgagaacatgatatatacatacttgtgaagtgtagttctatggttcacantnnaaaaaaaaaaaaaaaaaaaaaaaaaa

SEQ ID NO: 12 - DNA, 1481 bp, Artificial Sequence
  Expression homologous to A thaliana, AAD12260 subtilisin-like protease
ctccgatcgaaatcctaagtctaaattgccaagtttgctagagccagaaatctctcaagtgcccagaagccttgagcgcgaggtgcctggccttaatcagaaagctttattgatgaagaattgggaccaattccatctaagtggagagggatttaccaaaataattctgatcacaccttttagtgcaaaaggaagctaattggagcaaggtacttcaacgaaggatacgtgactctagcaagatctctcaattcaagtttctacacaccacgagacactgatggacatgtttcccacacgcagttcaaggatcaagtgtttcccggtatggaaatggaacagcaaagggtggatcaccaaaagtaagagtagcagcttacagagtttgctggcctccaattatgggcagtgggtgctttgattcagatatcttggttgcttttgatttggtaattgatgatggcgtggacgtgctttcagtctcacttggaggagatactggagcatatgtcaatgactctgtagctattggttcatttcatgttgttaagcacggcattgttgtcgttacctctgctggtaactcgtcctggtcccggtacaatacgaaaaaattgcaccttggctcataactgttggcgcgagaactatggattgtcagtttcccagctatatcattttaggaaacaaaaagcagtacaatttgaaacactgcccaaatgcatgttcttccctattataaatgttgcttcagcaaaagctccccatgcttcaactgacgatgctctcttatgcaaagctggggcattggacccaaagaaggtaaaggtaactattttagtttgtctaagaggagataatacgagggttgacaagggacagcaagctgctttggcaggtgcagttggaacgattctagccaacgattatgcatctggcgatgaaatttttgctgattctctctcgtcttacctgctacgcaaattagttacactgatggacttgaactctttagttcaacaagtatacctacagcttccattacacatccaacgacttaattgggaacaaagccagctccagtcatagcagccttttcatcaataggacctaacactgttacactggagatccttaaggttttacgcaggttcatgattttttgacacgacatcttttcttgaaagatcaggaaacgacatcttttcttgaaagatcaggaacttgaagtctgaagaagctatgcttgaagaatatgcggagcaagttgaagcacaatatagtttttgttctgttagtagcagtttaaagattttgtcatttgtacatcgtgtatagttctaatgtctgtttttaggagttgataaaaatagtgtcatgcaatttgcttaggtatatttattgacattgtgatccttcggttttttataatgaacaatgaaattttgtggaaaaaaaaaaaaaaaaaaaaaaaaaaaaaa

SEQ ID NO: 13 - DNA, 1193 bp, Artificial Sequence
  Expression homologous to Tomato, CAA07250 serine protease
gacagaattgaaaaaggacaagctgtaaagaacgctggaggcgttggcatgattctcatcaatcggcttcaggacggttcaactaaatcagccgatgctcatgtccttccggccctggatgtttcattttttgatggatttcaaattactgagtatatgaaatcaacaaagaatcctgttgctagaattacattccaaggaacgataataggcgataaaaatgctccagtgcttgctggtttttcatctcgcggaccaagcacagctagtcctggaatcttgaaacctgatattattggtcctggtgttaatgtcctagcagcttggcctacttctgtcgaaaacaaaaccaacaccaaatcaacattcaacataatttccggtacctctatgtcatgtcctcaccttagcggagttgcagcattgttaaaaagcgcgcaccctacttggtcccctgcagctattaaatcagcaatcatgacaaccgctgatacagtcaacctcgccaacaatcccatattagatgaaatgctccgtcctgcaaacatctttgccattggtgcaggacatgtcaatccatcacgagcaaatgatccaggactagtttacgatacacaattcaaggattacatatcttatttatgtggtttgaaatacacagatcgacagatgggaagccttctacaacgcagaacaagttgctcgaaagtgaaaagtattcctgaagcacaactcaattacccttcgttttccatttcacttggagcaaatcaacaaacatacacaagaacagtgacaaacgtcggggaggcaatgtcatcttatcgcgtgaagatagtttcaccacaaaatgtttccgttgttgttaagccttcaactctaaaatttacgaagttgaatcagaagttgacataccgagtgacattttccacaacaacaaacatcacaaacatggaagttgttcatggatacttgaaatggacaagtgataagcattttgtaagaagtccaattgctgttattctacaagagcatgaaacaccagaagattagtgtctttactttttaataatttgttcaatttataataaccccgtattaattgattgtatccaaaatgtagaatgagtgcaaaaattgctcatgttttattctactggtgatatttcccttgtggtaaaaaaaaaaaaaaa

SEQ ID NO: 14 - DNA, 748 bp, Artificial Sequence
  Expression homologous to Tomato, T06580 subtilisin-like proteinase p69f
ggcgtgattatcggagttatagacactggaattgttcctgaccatccttcatttagcgacgttgggatgcctcctccgcctgctaaatggaaaggattttgtgagtctaatttcacgaccaagtgtaacaacaaactcattggagccaggtctttcccgcttgacaatggtcccatagatgaaaatggacatggtacgcatacagcaagcacagctgcaggagcctttgtgaaaggtgctaatgtatttgggaatgccaatggaacagcagttggtgttgcccctcttgcgtacatagccatatataaggtatgcggttctgatggcgtttgttctgatgttgaaattttagctgcgatggatgtagctattgatgatggcgtagatattctatcaatatcccttggtggaactagtaatccgttccataatgacaagattgctcttggggcgtatagtgcaacagaaagaggtattcttgttagttgttctgcaggcaatagtggtccattccaacgcactgtagacaatgacgccccttggattctcacagttggcgctagcactcatgatagaaaactaaaggccactgttaagcttggaaataaagaagaatttgaaggagaatctgcttatcatccaaagacttcaaactcaacattcttcactctatttgatgttgaaaagatagtacacgagcaaccagtagcccctttctgcataccaggatcactcactgacccttcta

SEQ ID NO: 15 - DNA, 748 bp, Artificial Sequence
  Expression homologous to Tomato, T06580 subtilisin-like proteinase p69f
gggcgtgattatcggagttatagacactggaattgttcctgaccatccttcatttagcgacgttgggatgcctcctccgcctgctaaatggaaaggattttgtgagtctaatttcacgaccaagtgtaacaacaaactcattggagccaggtctttcccgcttgacaatggtcccatagatgaaaatggacatggtacgcatacagcaagcacagctgcaggagcctttgtgaaaggtgctaatgtatttgggaatgccaatggaacagcagttggtgttgcccctcttgcgtacatagccatatataaggtatgcggttctgatggcgtttgttctgatgttgaaattttagctgcgatggatgtagctattgatgatggcgtagatattctatcaatatcccttggtggaactagtaatccgttccataatgacaagattgctcttggggcgtatagtgcaacagaaagaggtattcttgttagttgttctgcaggcaatagtggtccattccaacgcactgtagacaatgacgccccttggattctcacagttggcgctagcactcatgatagaaaactaaaggccactgttaagcttggaaataaagaagaatttgaaggagaatctgcttatcatccaaagacttcaaactcaacattcttcactctatttgatgttgaaaagatagtacacgagcaaccagtagcccctttctgcataccaggatcactcactgacccttct

SEQ ID NO: 16 - DNA, 2538 bp, Artificial Sequence
  Expression homologous to A thaliana, BAB02339 cucumisin-like serine protease
cactttcttcgtcttcttcttctttctccctcttaatcttcttctttctcaactctttagtaatttcagtccaattggacggtcataaaactttcatagtacacgtgtccaaatcccataagccccacatctttactacccgccaacattggtactcctccatcctccgatcagtctcttcttcttcccaacactctgccaaaatcctttactcttacgattatgctgcccgtggtttctctgcccgtctcacttccgggcaggctgaccggctccgccgcatgcctggcgtggtctccgtcgtacctgaccgtgcacgtcagcttcacaccactcacacaccgaccttcttaggcctcgcagattcatttgggctttggcccaactccgattacgctgatgacgtcatcgtcggggtgctcgacacgggcatttggcccgaaaggccgagcttttccgacggcgggctttctgcagtcccttccggttggaaaggaaaatgcgaaactgggctggactttcctgcaacttcatgtaaccgtaaaatcatcggtgctcgattgttttacaaaggttacgaagctgatcgtggaagcccaattgacgaatctaaagaatctaaatcgccaagagatactgaaggacatgggactcacactgcttcaactgcagctggatctgttgtagctaacgctagtttttttcaatacgcaaaaggtgaagctagaggcatggctgtgaaagctcgaatagcagcttataagatctgttggaaaacagggtgttttgattctgatattttagctgcaatggatcaagctgttgctgatggagttcacgtgatttctctttccgttggcgctgacggttatgcaccggaatatgatgcggattctattgctattggagcttttggtgcttcagaacatggcgttgttgtctcttgctctgctggaaactccggtcccggtgcttccaccgcggtcaacgttgcgccgtggattctcaccgttgctgcttcaacgatagaccgggagtttccggctgatgttattttaggagatggcagaatattcggtggcgtatccctgtattccggcgatccgctcggggattcaaagctacctcttgtttactccggtgactgcgggagtcaactctgttatccaggaatgctggatccttcaaaggtagccggaaaaattgtattatgtgatcgaggcggcaatgctagagtagagaaaggaagtgcagtgaaattagccggcggtgcaggtatggtcctggcgaatttagctgactccggcgaagaactcgtcgccgattcacatctcctaccggcgacaatggtcggtcaaaaagccggtgacgaaataagggattacgtcaaatctgattcatcaccaaaagcgacgattgttttcaaaggaactgtaatcggaaaatcaccgtctgctccacgtattgctgcgttctcaggccgaggacccaattatgtaacaccggagatccttaaaccggatgttactgcaccaggagtcaacatattagccggttggaccgggtccataggaccaacagatttggaaattgataccagacgagtggaattcaacattatatctggtacatccatgtcttgtcctcatgttagcgggttagctgctttacttagaaaagcttaccctaaatggaccacagcagccatcaaatctgccctcatgacaacagcttacaacgttgataactccggcaaaacctttacagatctcgcgacaggccaggaatcgagtccgtttgttcacgggtcgggtcatgtggatccgaacagagcactagatccaggtcttgtctacgatattgacacgaaggattacgtggattttttatgcgccattggttatgatcccaaaagaatttcaccgttcgtgaaagatacttcttcagtgaattgcagcgaaaagaatttagttagtccgggggatttgaattatccatcgttctcagttgtatttggcagtgatagtgtggtgaaaaacaagcgtgtggttaaaaatgttggcaggaatacaaatgcggtgtatgaggtgaaaataaatgcgccgggttcggtggaggtgaaggtgactccgactaagcttagttttagcgagaaaaataagagtttgtcgtatgagattagttttagcagtaatggaagtgttgggttggagagagtaaaaggtcttgaatcagcatttgggtcaattgagtggagtgatggaattcacagcgtgaggagtccaattgcggtgcattggctactccactctgctacagaatctcagtgagcaatggactatgaagcaagaagataattgtgctaatctgcaaactgttatgggccagaaacaggaacaaggctaagttcagaaaggaaaaggaaatagggaaggaacatctatctgttgaaataatgttaagaaattttcatcattcttttcttgtttatgagtatttatcagccacaaaaaaaaaaaaaaaaaaaaaaaaaaaaaa

SEQ ID NO: 17 - DNA, 2426 bp, Artificial Sequence
  Expression homologous to Alnus glutinosa, S52769 subtilisin-like proteinase ag12
ggccaattgtattactatgtatttcttgctccttactatcttattacttactctaaatccattaactatggcagagtcagaaacttatatcatccatatggacttatcagccatgcctaaagctttttctagccatcagaattggtacttgaccactcttgcttctgtatcaggtagttcaagtcttggaactgaaagtaatagaaattccttttcctcatcaaaactagtatatgcttacactaacgctattcatggttttagtgcaactctttctccttctgagctacaagttataaaaaattctccaggctatctttcttcaactaaggacatgacagttaaaattgacacgacacacacgtctcaattccttggcctaaattccgattctggtgcatggccaaagtcagactatggcaaagatgttatagttggattagttgacacagggatttggccagagagtaaaagctataatgataatgggatgactgaagttccatcaagatggaaaggagaatgtgaaagtggaactcaatttaattcctctttatgcaacaagaaactcattggtgcgcgttacttcaacaaaggcctaattgccaataatccgaatattaccatctcgatgaattcagctcgtgacactgatgggcatggaactcacacatcctctacagctgcaggaagtcatgtagaatctgcatcttattttggctatgcgcgtggttctgctacagggatggcaccaaaggctcatgtggcaatgtacaaggctttgtgggaagaaggtacaatgttatctgatattctggctgcaattgatcaggcaattgaggatggagtggatataatatccttatcattaggcatagatgatcttgctttatatgaggatccggtagctattgccacatttgcagcaatggagaaagatatatttgtttccacttcagctggaaatgaagggcctgacgatcaggctttgcacaacggaacaccttgggttctaactgttgctgctggcacagttgatcgcgaatttatcgggacactaagtctgggtaatggagtttcagtcactggtttatctctctaccccgggaattccagttcaagcgaaagctccatcgtttttctcaagacatgcctagaggagaaggaactggagaaaaatgcacacaaattcgcagtctgctatgacacgaacggatcagtaagtgaccaattgtacaatgtaaaaaacacaaaagttgctggtggcatcttcataacaaattacactgacttggaattctacctccaaagcgaattcccggctgtgtttttgaactttgaagatggtgataaagttttggagtacatcaagaatagccattcaccaaaagcaaggcttgaatttcaagtgacacatcttggtgctaaaccagcaccaaaagttgctagctatagctcaaggggaccatcagaaagctgcccttttatcctcaaacctgacctgatggctcctggagccttaatattagcctcatggcctcaaaaatcaccggcaactcaaattcgctcaggagagcttttcagtaacttcaacatcatatcaggtacgtcaatgtcatgccctcatgcagctggtgtagcagcacttctgaaaggagcacaccccaaatggagtcctgctgccatccggtcggccatgatgaccacagccgacacgatggataacatgcaaatgcccatccgagacataggtcgcaacaataatgctgccagtcccctagccatgggagctggccgtatcaatccaaataaggcactagaccctggacttatctatgacattacatcacaggactatatcaatctcctctgtgctctagattttacatctcaacagataaaagccattacaaggtcctctgcttattcctgttccaactcatcattggatttaaactatccatcattcataggctatttcaattataacagcagcgagtcagaccctaaaaggatacaagaattccagaggacggtgactaatgtaggagaaggtatgtctgtatatacagccaaattgacctcaatgggtgattataaagctagtgttgcacctgacaagttggttttcaaagagaagtatgaaaagcaaagctacaagctaaggatagaaggtccattgctagttgatattatcttgtttatggttctttgagctgggtggaaactagcggtaaatatgttgtaaaaagtcccattgtcgcaactaccataagagtggatcctctgtgaggacagaactgattatgagtcctgtattctgaaaatgtgatacagtgatgaataattgtgaagttaaattcaaaaaaaaatcttttcagttagttaaaactaacttgctgattaaaaaaaaaaaaaaaaaaaaa

SEQ ID NO: 18 - protein, 737 aa, Nicotiana benthamiana
ANCITMYFLLLTILLLTLNPLTMAESETYIIHMDLSAMPKAFSSHQNWYLTTLASVSGSSSLGTESNRNSFSSSKLVYAYTNAIHGFSATLSPSELQVIKNSPGYLSSTKDMTVKIDTTHTSQFLGLNSDSGAWPKSDYGKDVIVGLVDTGIWPESKSYNDNGMTEVPSRWKGECESGTQFNSSLCNKKLIGARYFNKGLIANNPNITISMNSARDTDGHGTHTSSTAAGSHVESASYFGYARGSATGMAPKAHVAMYKALWEEGTMLSDILAAIDQAIEDGVDIISLSLGIDDLALYEDPVAIATFAAMEKDIFVSTSAGNEGPDDQALHNGTPWVLTVAAGTVDREFIGTLSLGNGVSVTGLSLYPGNSSSSESSIVFLKTCLEEKELEKNAHKFAVCYDTNGSVSDQLYNVKNTKVAGGIFITNYTDLEFYLQSEFPAVFLNFEDGDKVLEYIKNSHSPKARLEFQVTHLGAKPAPKVASYSSRGPSESCPFILKPDLMAPGALILASWPQKSPATQIRSGELFSNFNIISGTSMSCPHAAGVAALLKGAHPKWSPAAIRSAMMTTADTMDNMQMPIRDIGRNNNAASPLAMGAGRINPNKALDPGLIYDITSQDYINLLCALDFTSQQIKAITRSSAYSCSNSSLDLNYPSFIGYFNYNSSESDPKRIQEFQRTVTNVGEGMSVYTAKLTSMGDYKASVAPDKLVFKEKYEKQSYKLRIEGPLLVDIILFMVL

SEQ ID NO: 19 - protein, 289 aa, Nicotiana benthamiana
VSTQSAITAGDDGISEVPSRWKGECESGTEFNSSLCNKKLIGARYFNKGLLANNPNLNISMNSSRDTDGHGTHTSSTAAGSYVEGASYFGYATGTAIGIAPKAHVAMYKALWEEGVYLSDVLAAIDQAITDGVDVLSLSLGIDAIPLHEDPVAIAAFAALEKGIFVSTSAGNEGPYYETLHNGTPWVLTVAAGTVDREFIGTLTLGNGVSVPGLSLYPGNSSSSESSLVYVECQDDKELQKNAHKFVVCLDKNDSVGEHVYNVRNSKVAGAVFITNTTDLEFYLQSEFP

SEQ ID NO: 20 - protein, 683 aa, Nicotiana benthamiana
  misc_feature (7)..(7): Xaa can be any naturally occurring amino acid
TPRPTRXPTRPPTRPLSPSEYKAIKNSLGYVSSMEDRTVKIHTTHSSHFLGLSSMYGSWPKSNYGKGVIIGVVDTGVWPEIKSFDDDGMSQVPSRWKGICQTGTQFNSSLCNKKLIGARYFNKGLLSKVKNLTIMINSARDTEGHGTHTSSTAAGSLVKGASYFGYAPGFAIGVAPMAHVAVYKALWDGAGTISDILAALDQAIADGCDILSLSFGAVSPFPLYIDPISIASFSAMEKGIFVSVSAGNEGPFDQSLSNEAPWFLSVAASTVDRDVIRILTLGNGVSVTGLSLYPGNSTSDISVILVKNCLDKQELQNVTDKFVVCIDKNALVGKQVESVRHSNAAGAVFITNDFVTDLGEYLKTEFPSVFLNFQNGDQVLKYVNSTSSPKAKIGLQGTLIGVERAPAVAHFSSRGPSMTCPFILKPDLMAPGHLILASWSPLSSVSPYTELHNIFNIISGTSMSCPHAAGVAALVKGTHPEWSPAAIRSAMMTTADVLDNTQSPIQDIGRPENAAATPLAMGAGHINPNKAIDPGLIYDTTPQDYINLLCALNLTSEQIKTITRSSYTCPNPSLDLNYPSFIAYFNVNSSELDPTRVQEFKRTVTNVGEGVSEYTAELTAMPGLKVSVVPEKLVFKDKYEKQSYKLRIECPQLMNDFLVHGSLSWVEKGGKYVVRSPIVATNS

SEQ ID NO: 21 - protein, 770 aa, Nicotiana benthamiana
  misc_feature (211)..(211): Xaa can be any naturally occurring amino acid
VFPFFFIIISFCLTPVTISVQSDGHETFIIHVSKSDKPRVFTTHHHWYSSIIRSVSQHPSKILYTYERAAVGFSARLTAAQADQLRRIPGVISVLPDEVRHLHTTHTPTFLGLADSFGLWPNSDYADDVIVGVLDTGIWPERPSFSDEGLSTVPSSWKGKCVTGPDFPETSCNKKIIGAQMFYKGYEAKHGPMDESKESKSPRDTEGHGTXTASTAAGSLVANASFYQYAKGEARGMAIKARIAAYKICWKNGCFNSDILAAMDQAVDDGVHVISLSVGANGYAPHYLYDSIAIGAFGASEHGVLVSCSAGNSGPGAYTAVNIAPWMLTVGASTIDREFPADVILGDNRIFGGVSLYSGNPLTDAKLPVVYSGDCGSKYCYPGKLDPKKVAGKIVLCDRGGNARVEKGSAVKQAGGVGMILANLADSGEELVADSHLLPATMVGQKAGDKIRHYVTSDPSPTATIVFRGTVIGKSPAAPRVAAFSSRGPNHLTPEILKPDVIAPGVNILAGWTGSVGPTDLDIDTRRVEFNIISGTSMSCPHVGGLAALLRRAHPKWTPAAVKSALMTTAYNLDNSGKVFTDLATGQESTPFVHGSGHVDPNRALDPGLIYDIETSDYVNFLCSIGYDGDDVAVFARDSSRVNCSERSLATPGDLNYPSFSVVFTGESNGVVKYKRVVNNVGKNTDAVYEVKVNAPSSVEVNVSPAKLVFSEEKQSLSYEISLKSKKSGDLQMVKGIESAFGSIEWSDGIHNVRSPIAVRWRHYSDAASM

SEQ ID NO: 22 - protein, 770 aa, Nicotiana benthamiana
PTRPVFPFFFIIISFCLTPVTISVQSDGHETFIIHVSKSDKPRVFTTHHHWYSSIIRSVSQHPSKILYTYERAAVGFSARLTAAQADQLRRIPGVISVLPDEVRHLHTTHTPTFLGLADSFGLWPNSDYADDVIVGVLDTGIWPERPSFSDEGLSTVPSSWKGKCVTGPDFPETSCNKKIIGAQMFYKGYEAKHGPMDESKESKSPRDTEGHGTHTASTAAGSLVANASFYQYAKGMAIKARIAAYKICWKNGCFNSDILAAMDQAVDDGVHVISLSVGANGYAPHYLYDSIAIGAFGASEHGVLVSCSAGNSGPGAYTAVNIAPWMLTVGASTIDREFPADVILGDNRIFGGVSLYSGNPLTDAKLPVVYSGDCGSKYCYPGKLDPKKVAGKIVLCDRGGNARVEKGSAVKQAGGVGMILANLADSGEELVADSHLLPATMVGQKAGDKIRHYVTSDPSPTATIVFRGTVIGKSPAAPRVAAFSSRGPNHLTPEILKPDVIAPGVNILAGWTGSVGPTDLDIDTRRVEFNIISGTSMSCPHVGGLAALLRRAHPKWTPAAVKSALMTTAYNLDNSGKVFTDLATGQESTPFVHGSGHVDPNRALDPGLIYDIETSDYVNFLCSMAYDGDDVAVFARDSSRVNCSERSLATPGDLNYPSFSVVFTGESNGVVKYKRVVNNVGKNTDAVYEVKVNAPSSVEVNVSPAKLVFSEEKQSLSYEISLKSKKSGDLQMVKGIESAFGSIEWSDGIHNVRSPIAVRWRHYSDAASM

SEQ ID NO: 23 - protein, 775 aa, Nicotiana benthamiana
LSSSSSSFSLLIFFFLNSLVISVQLDGHKTFIVHVSKSHKPHIFTTRQHWYSSILRSVSSSSQHSAKILYSYDYAARGFSARLTSGQADRLRRMPGVVSVVPDRARQLHTTHTPTFLGLADSFGLWPNSDYADDVIVGVLDTGIWPERPSFSDGGLSAVPSGWKGKCETGLDFPATSCNRKIIGARLFYKGYEADRGSPIDESKESKSPRDTEGHGTHTASTAAGSVVANASFFQYAKGEARGMAVKARIAAYKICWKTGCFDSDILAAMDQAVADGVHVISLSVGADGYAPEYDADSIAIGAFGASEHGVVVSCSAGNSGPGASTAVNVAPWILTVAASTIDREFPADVILGDGRIFGGVSLYSGDPLGDSKLPLVYSGDCGSQLCYPGMLDPSKVAGKIVLCDRGGNARVEKGSAVKLAGGAGMVLANLADSGEELVADSHLLPATMVGQKAGDEIRDYVKSDSSPKATIVFKGTVIGKSPSAPRIAAFSGRGPNYVTPEILKPDVTAPGVNILAGWTGSIGPTDLEIDTRRVEFNIISGTSMSCPHVSGLAALLRKAYPKWTTAAIKSALMTTAYNVDNSGKTFTDLATGQESSPFVHGSGHVDPNRALDPGLVYDIDTKDYVDFLCAIGYDPKRISPFVKDTSSVNCSEKNLVSPGDLNYPSFSVVFGSDSVVKNKRVVKNVGRNTNAVYEVKINAPGSVEVKVTPTKLSFSEKNKSLSYEISFSSNGSVGLERVKGLESAFGSIEWSDGIHSVRSPIAVHWLLHSATESQ

SEQ ID NO: 24 - protein, 398 aa, Nicotiana benthamiana
GVIIGVIDTGIFPDHPSFSDVGMSPPPAKWKGFCESNFTTKCNNKIIGLRSFRLSEDTPIDTDGHGTHTASTAAGAFVKGANFFGNANGTAVGVAPLAHMAIYKVCSFATCSETDALAAMDAAIDDGVDIISASLGGFTNAPLHDDPISLGAYSATEKGILASASAGNSEFDNPVANNAPWILTVGASTHDRKLKATVKLGNKEEFEGESADQPKTSNSTFIALFDAGKNASDQDAPFCRSWAMTDPAIKGKIVLCQKDPSSLTSSQGRNVKDAGGVGMILINNPEDGVTKSATAHVLPALDVSHEEGEKIKAYINSTSNPIAAITFQGTVIGDKNAPIVASFSARGPSRANPGILKPDIIGPGVNILAAWPTTVNIPNKNTNSGFNIISGTSMSCPH

SEQ ID NO: 25 - protein, 398 aa, Nicotiana benthamiana
GVIIGVIDTGIVPDHPSFSDVGMPPPPAKWKGFCESNFTTKRNNKLIGARSFPLDNGPIDENGHGTHTASTAAGAFVKGANVFGNANGTAVGVAPLAHIAIYKVCGSDGVCSDVEILPAMDVAIDDGVDILSISLGGTSNPFHNDKIALGAYSATERGILVSCSAGNSGPFQRTVNNDAPWILTVGASTHDRKLKATVKLGNKEEFEGESAYHPKTSSSTFFTLFDVEKDGTRATRAPFCIPGSLTDPSIRGKIVVCLVGGGVRTVDKGQVVKDAGGVGMILINNPEDGVTKSAEAHVLPALDVSDADGKKILAYINSTSNPVAAITFHGTVLGDKNAPIVASFSSRGPSEASRGILKPDIIGPGVNVLAAWPTSVDNNKNTKSTFNIISGTSMSCPH

SEQ ID NO: 26 - protein, 166 aa, Nicotiana benthamiana
HASDGAGHINPRKAVDPGLVYDIGAQDYFEFLCTQQLSPSQLTVFGKFSNRTCHHSLANPGDLNYPAISAVFPEDAKVSTLTLHRTVTNVGSPISNYHVRVSPFKGAVVKVEPSRLNFTSKHQKLSYKVIFETKYRQKAREFGSLLWKDGTHKVRSTIVITWLASI

SEQ ID NO: 27 - protein, 766 aa, Nicotiana benthamiana
  misc_feature (455)..(455): Xaa can be any naturally occurring amino acid
MARPGGMVLSTLFLMLFHVFVHAGQNQKKTYIIYMDKSNIPADFDDHTLWYDSSLKSVSKGANMLYTYNNVIHGYSTQLTADEAKSLEQQPGILSVHEEVRYELHTTRSPTFLGLEGRESKSFFLQAETRSEVIIGVLDTGVWPESKSFDDTGLGPVPMSWKGECQIGKNFKASSCNRKLIGARFFSQGYEAAFGAIDETTESKSPRDDDGHGTHTATTAAGSVVTGASLFGYAAGTARGMASHARVAAYKVCWAGGCFSSDILAGMDQAVIDGVNVLSLSLGGTISDYYRDIVAIGGFSAASQGIFVSCSAGNGGPGSGSLSNAAPWITTVGAGTMDREFPAYISLGNGKKFSGVSLYSGKALPSSVMPLVYAGNASQASNGNLCTSGSLIPEKVDGKIVVCDRGMNARAQKGLVVKDAGGIGMILANTDSYGDELVADAHLIPTGAVGQTAGXLIXRYIASDSNPITTIAFGGTKLGVQPSPVVAAFSSRGPNPITPEILKPDLIAPGVNILAGWTGKVGPTGLPEDTRNVGFNIISGTSMSCXHVSGLAAXLXAAHPEWSXGVIRSALMTTGYSTHKNGXMIEDVATGMSYTPVDHGAGHVNPAAAMNPGLXYDLTVDDYINFLCALDYSPSMIKVIAKRDISCXNNKDIELLTLITHLLPFLWKRAWGEHANSSAPTVTRYTRTLTNVGNPATYKASVSSEMQEVKIQVEPQTLTFSRKKEKKTYTVTFTASSKPSGTTSFARLEWSDGQHVVASPIAFSWT

SEQ ID NO: 28 - protein, 350 aa, Nicotiana benthamiana
DRIEKGQAVKNAGGVGMILINRLQDGSTKSADAHVLPALDVSFFDGFQITEYMKSTKNPVARITFQGTIIGDKNAPVLAGFSSRGPSTASPGILKPDIIGPGVNVLAAWPTSVENKTNTKSTFNIISGTSMSCPHLSGVAALLKSAHPTWSPAAIKSAIMTTADTVNLANNPILDEMLRPANIFAIGAGHVNPSRANDPGLVYDTQFKDYISYLCGLKYTDRQMGSLLQRRTSCSKVKSIPEAQLNYPSFSISLGANQQTYTRTVTNVGEAMSSYRVKIVSPQNVSVVVKPSTLKFTKLNQKLTYRVTFSTTTNITNMEVVHGYLKWTSDKHFVRSPIAVILQEHETPED

SEQ ID NO: 29 - protein, 181 aa, Nicotiana benthamiana
AITAGHVNPESAIDPGLIYDTDTSDYINLLCSLNYTEKEMKLFTNESNPCSGFTGSPLDLNYPSLSVMFRPDSSVHVVKRTLTHVAVSKPEVYKVKILNLNSEKVSLSISPMELMFNESLRKQRYMVKFESHHIFNSSRKIAEQMAFGSISWESEKHNVRSPFAVMWVQQNFNNSRLYKIT

SEQ ID NO: 30 - protein, 10 aa, Nicotiana benthamiana
TTHTSQFLGL

SEQ ID NO: 31 - protein, 14 aa, Nicotiana benthamiana
FGYATGTAIGIAPK

SEQ ID NO: 32 - DNA, 24 bp, Artificial Sequence
  Used in PCR
gtcctaatccctagggatttaagg

SEQ ID NO: 33 - DNA, 19 bp, Artificial Sequence
  Used in PCR
ctttggaaattgcagaaac

SEQ ID NO: 34 - DNA, 19 bp, Artificial Sequence
  Used in PCR
gtttctgcaatttccaaag

SEQ ID NO: 35 - DNA, 45 bp, Artificial Sequence
  Used in PCR
gaattcggggtaccgcggccgcgatatcctgcagggcgttaactc

SEQ ID NO: 36 - DNA, 45 bp, Artificial Sequence
  Used in PCR
gaattcggtaccctgcaggatatcgcggccgcggcgttaactcgg

SEQ ID NO: 37 - DNA, 42 bp, Artificial Sequence
  Used in PCR
tggttctgcagttatgcataggcgtgattatcggagttatag

SEQ ID NO: 38 - DNA, 37 bp, Artificial Sequence
  Used in PCR
tttccttttgcggccgcgtgagggcaagacattgatg

SEQ ID NO: 39 - protein, 249 aa, Nicotiana benthamiana
GVIIGVIDTGIVPDHPSFSDVGMPPPPAKWKGFCESNFTTKCNNKLIGARSFPLDNGPIDENGHGTHTASTAAGAFVKGANVFGNANGTAVGVAPLAYIAIYKVCGSDGVCSDVEILAAMDVAIDDGVDILSISLGGTSNPFHNDKIALGAYSATERGILVSCSAGNSGPFQRTVDNDAPWILTVGASTHDRKLKATVKLGNKEEFEGESAYHPKTSNSTFFTLFDVEKIVHEQPVAPFCIPGSLTDPS

SEQ ID NO: 40 - protein, 249 aa, Nicotiana benthamiana; identical to SEQ ID NO: 39